(19) United States
(12) Patent Application Publication
(10) Pub. No.: US 2003/0207877 A1
(43) Pub. Date: Nov. 6, 2003

(54) Pyridoquinoxaline antivirals

(76) Inventors: Joseph Walter Strohbach, Mendon, MI (US); Steven P. Tanis, Kalamazoo, MI (US); Malcolm Wilson Moon, Kalamazoo, MI (US); James A. Nieman, Galesburg, MI (US); Thomas J. Beauchamp, Portage, MI (US); Jill M. Northuis, Portage, MI (US); William D. McGhee, Fenton, MO (US)

Correspondence Address: PHARMACIA & UPJOHN, 301 HENRIETTA ST, 0228-32-LAW, KALAMAZOO, MI, 49007, US

(21) Appl. No.: 10/326,036

(22) Filed: Dec. 19, 2002

Related U.S. Application Data
(60) Provisional application No. 60/342,876, filed on Dec. 20, 2001.

(51) Int. Cl. A61K 31/541; A61K 31/5377; A61K 31/498; C07D 487/02

(52) U.S. Cl. 514/228.2; 514/233.5; 514/250; 544/60; 544/120; 544/344

(57) ABSTRACT

The present invention provides a compound of formula I

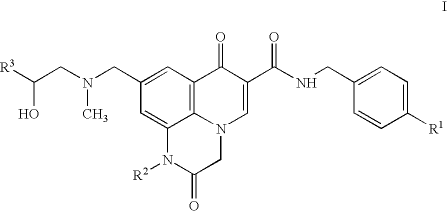

or a pharmaceutically acceptable salt thereof wherein R¹, R² and R³ are as defined in the specification. The compounds are useful for the treatment of viral infections.

### FIELD OF THE INVENTION

The present invention provides pyridoquinoxalines that are useful as antiviral agents. More specifically, it provides compounds of formula I described herein below against herpesviruses.

### BACKGROUND OF THE INVENTION

The herpesviruses comprise a large family of double stranded DNA viruses. They are also a source of the most common viral illnesses in man. Eight of the herpes viruses, herpes simplex virus types 1 and 2 (HSV-1 and HSV-2), varicella zoster virus (VZV), human cytomegalovirus (HCMV), Epstein-Barr virus (EBV), and human herpes viruses 6, 7, and 8 (HHV-6, HHV-7, and HHV-8), have been shown to infect humans.

HSV-1 and HSV-2 cause herpetic lesions on the lips and genitals, respectively. They also occasionally cause infections of the eye and encephalitis. HCMV causes birth defects in infants and a variety of diseases in immunocompromised patients such as retinitis, pneumonia, and gastrointestinal disease. VZV is the causitive agent of chicken pox and shingles. EBV causes infectious mononucleosis. It can also cause lymphomas in immunocompromised patients and has been associated with Burkitt's lymphoma, nasopharyngeal carcinoma, and Hodgkins disease. HHV-6 is the causative agent of roseola and may be associated with multiple sclerosis and chronic fatigue syndrome. HHV-7 disease association is unclear, but it may be involved in some cases of roseola. HHV-8 has been associated with Karposi's sarcoma, body cavity based lymphomas, and multiple myeloma.

Infection by reactivation of herpesviruses is associated with several cardiovascular diseases or conditions in the host such as atherosclerosis and restenosis resulting in inflammation of coronary vessel walls. It is thought that in many patients suffering from restenosis following coronary atherectomy viral infection particularly by CMV plays an important role in the proliferation of the disease. Atherosclerosis is believed to be be associated with the overall infectious disease burden in the host and particularly by the herpesviruses such as HSV, CMV, and EBV.

Infection in the animal population (livestock and companion) by strains of herpesviruses is endemic including cattle (Bovine herspesvirus 1-5, BHV), sheep (Ovine herpesvirus 1 and 2), dog (Canine herpesvirus 1), horse (Equine herpesvirus 1-8, EHV), cat (Feline herpesvirus 1, FHV), swine (pseudorabies virus, PRV), and many species of fowl. In the case of bovine herpesvirus infection, animals may suffer from ocular, respiratory, or digestive disorders. Pseudorabies is an extremely contagious viral pathogen infecting several species such as cattle, horses, dogs, cats, sheep, and goats leading to rapid death. The virus is benign in adult swine, however, it remains contagious and leads to high mortality in pigs under three weeks. Infection of horses by equine herpesvirus may lead to neurological syndromes, respiratory disease, and neonatal disease. Herpesvirus infection in cats leads to the disease known as feline viral rhinotracheitis (FVR) which is characterized by rhinitis, tracheitis, laryngitis, and conjunctivitis.

It has been surprisingly discovered that compounds of the present invention demonstrate greatly enhanced oral bioavailability and improved selectivity for the viral targets.

### INFORMATION DISCLOSURE

U.S. Pat. No. 5,792,774 discloses specific quinoline derivatives that are alleged to have therapeutic utility via inhibition of Phosphodiesterase IV esterase and/or Tumor Necrosis factor activity.

PCT/US01/16494 discloses heterocycle carboxamides as antiviral agents.

Despite the above teachings, there still exists a need in the art for compounds that have enhanced oral bioavailability and improved selectivity for the viral targets.

### SUMMARY OF THE INVENTION

The present invention provides a compound of formula I, A compound of formula I

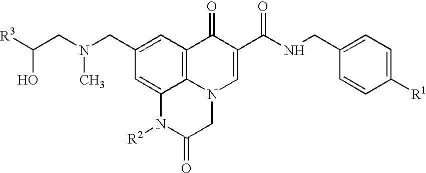

or a pharmaceutically acceptable salt thereof

wherein R¹ is F or Cl;

R² is C₁₋₄alkyl, optionally substituted by OH or OC₁₋₄alkyl;

R³ is aryl or heteroaryl, optionally substituted by one to three C₁₋₂alkyl, OH, OC₁₋₂alkyl or CN;

aryl is a phenyl or benzyl radical optionally fused to a benzene ring; and

heteroaryl is a 5- or 6-membered aromatic ring having at least one heteroatom selected from the group consisting of O, S and N(X) wherein X is absent or H, wherein heteroaryl is optionally fused to a benzene ring.

The present invention further provides:

a pharmaceutical composition comprising a compound of formula I or a pharmaceutically acceptable salt thereof, and a pharmaceutically acceptable carrier;

a method of treating or preventing a herpesviral infection comprising administering to a mammal in need of such treatment, a compound of formula I or a pharmaceutically acceptable salt thereof, wherein the method is administered orally, parenterally, topically, rectally, nasally, sublingually or transdermally;

a method for the treatment of atherosclerosis and restenosis comprising administering to a mammal in need of such treatment, a compound of formula I or a pharmaceutically acceptable salt thereof, wherein the method is administered orally, parenterally, topically, rectally, nasally, sublingually or transdermally;

a method for the treatment of herpesviral infections comprising administering a composition comprising a pharmaceutically effective amount of the compound of formula I and at least one other antiviral agent;

a method for the treatment of atherosclerosis and restenosis comprising administering a composition comprising a pharmaceutically effective amount of the compound of formula I and at least one other antiviral agent;

a pharmaceutically acceptable salt thereof for use in medical treatment;

a use of a compound of formula I or a pharmaceutically acceptable salt thereof to prepare a medicament for treating or preventing a herpesviral infection in a mammal; and

a method for inhibiting a viral DNA polymerase, comprising contacting (in vitro or in vivo) the polymerase with an effective inhibitory amount of a compound of formula I, or a pharmaceutically acceptable salt thereof.

The invention also provides novel intermediates and processes disclosed herein that are useful for preparing compounds of formula I.

### DETAILED DESCRIPTION OF THE INVENTION

The following definitions are used, unless otherwise described. Alkyl denotes both straight and branched groups; but reference to an individual radical such as “propyl” embraces only the straight chain radical, a branched chain isomer such as “isopropyl” being specifically referred to.

The carbon atom content of various hydrocarbon-containing moieties is indicated by a prefix designating the minimum and maximum number of carbon atoms in the moiety, i.e., the prefix Cᵢ₋ⱼ indicates a moiety of the integer “i” to the integer “j” carbon atoms, inclusive. Thus, for example, (C₁₋₄)alkyl refers to alkyl of one to four carbon atoms, inclusive, or methyl, ethyl, propyl, isopropyl and butyl, straight and branched forms thereof. Representative heteroaryls include pyrazinyl; furanyl; thienyl; pyridyl; pyrimidinyl; isoxazolyl; isothiazolyl; oxazolyl; thiazolyl; pyrazolyl; furazanyl; pyrrolyl; pyrazolyl; triazolyl; 1,2,4-thiadiazolyl; pyrazinyl; pyridazinyl; quinoxalinyl; phthalazinyl; 1(2H)-phthalazinonyl; imidazopyridinyl; imidazothiazolyl; benzofurazanyl indolyl; azaindolyl; benzimidazolyl; benzothienyl; quinolinyl; imidazolyl; thienopyridyl; quinazolinyl; thienopyrimidyl; pyrrolopyridyl; imidazopyridyl; isoquinolinyl; benzoazaindolyl; azabenzimidazolyl; 1,2,4-triazinyl; benzothiazolyl; furopyridyl and the like.

Mammal denotes human and animals. Animals specifically refers to food animals or companion animals.

The term “antiviral agent” refers to an antiviral drug other than a compound of formula I. Specifically, they refer to Acyclovir, Penciclovir, Famiciclovir, Valaciclovir, Ganciclovir, Valganciclovir, Foscarnet, and Cidofovir. Such antiviral agents can be either obtained commercially or be prepared according to the references cited in PHYSICIANS' DESK REFERENCE, the 54^{th} Edition (2000) and the US FDA's Orange book.

Compounds of the invention may have one or more chiral centers and be isolated in optically active and racemic forms. Some compounds may exhibit polymorphism. It is to be understood that the present invention encompasses any racemic, optically-active, polymorphic, tautomeric, or stereoisomeric form, or mixture thereof, of a compound of the invention, which possesses the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine antiviral activity using the standard tests described herein, or using other similar tests which are well known in the art.

The compounds of the present invention are generally named according to the IUPAC or CAS nomenclature system. Abbreviations which are well known to one of ordinary skill in the art may be used (e.g. “Ph” for phenyl, “Me” for methyl, “Et” for ethyl, “h” for hour or hours and “rt” for room temperature, “N” for nitrogen atom, “O” for oxygen atom, and “S” for sulfur atom).

Specifically, R¹ is chloro.

Specifically, R¹ is fluoro.

Specifically, R² is methyl.

Specifically, R² is ethyl.

Specifically, R² is ethyl substituted by OH.

Specially, R³ is phenyl.

Specifically, R³ is phenyl substituted by one or two OH or OCH₃.

Specifically, R³ is a 5-membered aromatic ring having at least one heteroatom selected from the group consisting of O, S and N(X) wherein X is absent or H.

Specifically, R³ is furyl, thienyl or thiazol, wherein R³ is optionally fused to a benzene ring.

Specifically, R³ is optionally substituted by one to two methyl, OH, OCH₃ or CN.

Specifically, R³ is 1-benzofuran-2-yl, 3-furyl, 2-furyl, 3-thienyl, 5-methyl-2-furyl, 2,5-dimethyl-3-furyl, 2-thienyl, 1-benzothien-3-yl, 5-cyanothien-2-yl, or 1,3-thiazol-2yl.

Specifically, R³ is a 6-membered aromatic ring having at least one heteroatom selected from the group consisting of O, S and N.

Specifically, R³ is pyridinyl, pyrimidinyl, or pyrazinyl, wherein wherein R³ is optionally fused to a benzene ring.

Specifically, R³ is optionally substituted by one to two methyl, OH, OCH₃ or CN.

Specifically, R³ is pyridin-2-yl, 6-methylpyridin-2-yl, pyridin-3-yl, quinolin-2-yl, or pyrimidin-2-yl.

Specifically, compounds of formula I includes enantionmers of formula IA:

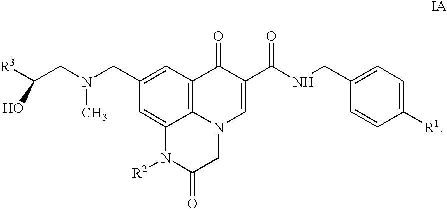

Specifically, compounds of formula I includes enantionmers of formula IB:

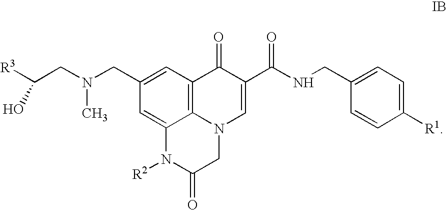

Examples of the present invention are:

(1) N-(4-chlorobenzyl)-9-{[(2-hydroxy-2-phenylethyl) (methyl)amino]methyl}-1-methyl-2,7-dioxo-2,3-dihydro-1H,7H-pyrido[1,2,3-de]quinoxaline-6-carboxamide,

(2) rac N-(4-chlorobenzyl)-9-{[[2-hydroxy-2-(4-hydroxyphenyl)ethyl](methyl)amino]methyl}-1-methyl-2,7-dioxo-2,3-dihydro-1H,7H-pyrido [1,2,3-de]quinoxaline-6-carboxamide,

(3) rac 9-{[[2-(1-benzofuran-2-yl)-2-hydroxyethyl](methyl)amino]methyl}-N-(4-chlorobenzyl)-1-methyl-2,7-dioxo-2,3-dihydro-1H,7H-pyrido [1,2,3-de]quinoxaline-6-carboxamide,

(4) rac N-(4-chlorobenzyl)-9-{[[2-hydroxy-2-(5-methyl-2-furyl)ethyl](methyl)amino]methyl}-1-methyl-2,7-dioxo-2,3-dihydro-1H,7H-pyrido [1,2,3-de]quinoxaline-6-carboxamide,

(5) N-(4-chlorobenzyl)-9-{[[(2R)-2-(2-furyl)-2-hydroxyethyl](methyl)amino]methyl}-1-methyl-2,7-dioxo-2,3-dihydro-1H,7H-pyrido[1,2,3-de]quinoxaline-6-carboxamide,

(6) N-(4-chlorobenzyl)-9-{[[(2S)-2-hydroxy-2-phenylethyl](methyl)amino]methyl}-1-methyl-2,7-dioxo-2,3-dihydro-1H,7H-pyrido[1,2,3-de]quinoxaline-6-carboxamide,

(7) N-(4-chlorobenzyl)-9-{[[(2S)-2-hydroxy-2-pyridin-3-ylethyl](methyl)amino]methyl}-1-methyl-2,7-dioxo-2,3-dihydro-1H,7H-pyrido[1,2,3-de]quinoxaline-6-carboxamide,

(8) rac 9-{[[2-(1-benzothien-3-yl)-2-hydroxyethyl](methyl)amino]methyl 1-N-(4-chlorobenzyl)-1-methyl-2,7-dioxo-2,3-dihydro-1H,7H-pyrido[1,2,3-de]quinoxaline-6-carboxamide,

(9) rac N-(4-chlorobenzyl)-9-{[(2-hydroxy-2-quinolin-2-ylethyl) (methyl)amino]methyl}-1-methyl-2,7-dioxo-2,3-dihydro-1H,7H-pyrido[1,2,3-de]quinoxaline-6-carboxamide,

(10) N-(4-chlorobenzyl)-9-{[[(2R)-2-hydroxy-2-pyrazin-2-ylethyl](methyl)amino]methyl}-1-methyl-2,7-dioxo-2,3-dihydro-1H,7H-pyrido[1,2,3-de]quinoxaline-6-carboxamide,

(11) rac 9-{[[2-(1-benzofuran-2-yl)-2-hydroxyethyl](methyl)amino]methyl}-N-(4-fluorobenzyl)-1-methyl-2,7-dioxo-2,3-dihydro-1H,7H-pyrido[1,2,3-de]quinoxaline-6-carboxamide,

(12) 9-{[[(2R)-2-(1-benzofuran-2-yl)-2-hydroxyethyl](methyl)amino]methyl}-N-(4-chlorobenzyl)-1-methyl-2,7-dioxo-2,3-dihydro-1H,7H-pyrido[1,2,3-de]quinoxaline-6-carboxamide, or

(13) 9-([[(2R)-2-(1-benzofuran-2-yl)-2-hydroxyethyl](methyl)amino]methyl}-N-(4-fluorobenzyl)-1-methyl-2,7-dioxo-2,3-dihydro-1H,7H-pyrido[1,2,3-de]quinoxaline-6-carboxamide.

Other examples of the present invention are:

(1) rac N-(4-chlorobenzyl)-9-{[(2-hydroxy-2-phenylethyl)(methyl)amino]methyl}-1-methyl-2,7-dioxo-2,3-dihydro-1H,7H-pyrido[1,2,3-de]quinoxaline-6-carboxamide,

(2) rac N-(4-chlorobenzyl)-9-{[[2-(3-furyl)-2-hydroxyethyl](methyl)amino]methyl{-1-methyl-2,7-dioxo-2,3-dihydro-1H,7H-pyrido[1,2,3-de]quinoxaline-6-carboxamide,

(3) rac N-(4-chlorobenzyl)-9-{[[2-(2-furyl)-2-hydroxyethyl](methyl)amino]methyl}-1-methyl-2,7-dioxo-2,3-dihydro-1H,7H-pyrido[1,2,3-de]quinoxaline-6-carboxamide,

(4) rac N-(4-chlorobenzyl)-9-{[[2-(2,5-dimethyl-3-furyl)-2-hydroxyethyl](methyl)amino]methyl}-1-methyl-2,7-dioxo-2,3-dihydro-1H,7H-pyrido[1,2,3-de]quinoxaline-6-carboxamide,

(5) rac N-(4-chlorobenzyl)-9-[[2-hydroxy-2-(6-methylpyridin-2-yl)ethyl](methyl)amino]methyl}-1-methyl-2,7-dioxo-2,3-dihydro-1H,7H-pyrido[1,2,3-de]quinoxaline-6-carboxamide,

(6) N-(4-chlorobenzyl)-9-{[[(2R)-2-hydroxy-2-pyridin-2-ylethyl](methyl)amino]methyl}-1-methyl-2,7-dioxo-2,3-dihydro-1H,7H-pyrido[1,2,3-de]quinoxaline-6-carboxamide,

(7) rac N-(4-chlorobenzyl)-9-{[[2-hydroxy-2-(4-hydroxy-3-methoxyphenyl)ethyl](methyl)amino]methyl}-1-methyl-2,7-dioxo-2,3-dihydro-1H,7H-pyrido[1,2,3-de]quinoxaline-6-carboxamide,

(8) N-(4-chlorobenzyl)-9-[[(2S)-2-hydroxy-2-thien-3-ylethyl](methyl)amino]methyl}-1-methyl-2,7-dioxo-2,3-dihydro-1H,7H-pyrido[1,2,3-de]quinoxaline-6-carboxamide,

(9) rac N-(4-chlorobenzyl)-9-{[(2-hydroxy-3-phenylpropyl)(methyl)amino]methyl}-1-methyl-2,7-dioxo-2,3-dihydro-1H,7H-pyrido[1,2,3-de]quinoxaline-6-carboxamide, (10) rac N-(4-chlorobenzyl)-9-{[[2-hydroxy-2-(3-methoxyphenyl)ethyl](methyl)amino]methyl}-1-methyl-2,7-dioxo-2,3-dihydro-1H,7H-pyrido[1,2,3-de]quinoxaline-6-carboxamide,

(11) rac N-(4-chlorobenzyl)-9-{[(2-hydroxy-2-pyrimidin-2-ylethyl)(methyl)amino]methyl}-1-methyl-2,7-dioxo-2,3-dihydro-1H,7H-pyrido[1,2,3-de]quinoxaline-6-carboxamide,

(12) rac N-((4-chlorobenzyl)-9-{[[2-(5-cyanothien-2-yl)-2-hydroxyethyl](methyl)amino]methyl}-1-methyl-2,7-dioxo-2,3-dihydro-1H,7H-pyrido[1,2,3-de]quinoxaline-6-carboxamide,

(13) N-(4-chlorobenzyl)-9-{[[(2R)-2-hydroxy-2-(1,3-thiazol-2-yl)ethyl](methyl)amino]methyl}-1-methyl-2,7-dioxo-2,3-dihydro-1H,7H-pyrido[1,2,3-de]quinoxaline-6-carboxamide,

(14) N-(4-fluorobenzyl)-9-{[[(2R)-2-(2-furyl)-2-hydroxyethyl](methyl)amino]methyl}-1-methyl-2,7-dioxo-2,3-dihydro-1H,7H-pyrido[1,2,3-de]quinoxaline-6-carboxamide,

(15) N-(4-fluorobenzyl)-9-{[[(2R)-2-hydroxy-2-pyridin-2-ylethyl](methyl)amino]methyl}-1-methyl-2,7-dioxo-2,3-dihydro-1H,7H-pyrido[1,2,3-de]quinoxaline-6-carboxamide,

(16) N-(4-fluorobenzyl)-9-{[[(2S)-2-hydroxy-2-pyridin-3-ylethyl](methyl)amino]methyl}-1-methyl-2,7-dioxo-2,3-dihydro-1H,7H-pyrido[1,2,3-de]quinoxaline-6-carboxamide,

(17) rac 9-{[[2-(1-benzofuran-2-yl)-2-hydroxyethyl](methyl)amino]methyl}-N-(4-chlorobenzyl)-1-(2-hydroxyethyl)-2,7-dioxo-2,3-dihydro-1H,7H-pyrido[1,2,3-de]quinoxaline-6-carboxamide,

(18) N-(4-fluorobenzyl)-9-{[[(2R)-2-hydroxy-2-(1,3-thiazol-2-yl)ethyl](methyl)amino]methyl}-1-methyl-2,7-dioxo-2,3-dihydro-1H,7H-pyrido[1,2,3-de]quinoxaline-6-carboxamide,

(19) N-(4-fluorobenzyl)-9-{[[(2S)-2-hydroxy-2-thien-3-ylethyl](methyl)amino]methyl}-1-methyl-2,7-dioxo-2,3-dihydro-1H,7H-pyrido[1,2,3-de]quinoxaline-6-carboxamide,

(20) N-(4-fluorobenzyl)-9-{[[(2S)-2-hydroxy-2-phenylethyl](methyl)amino]methyl}-1-methyl-2,7-dioxo-2,3-dihydro-1H,7H-pyrido[1,2,3-de]quinoxaline-6-carboxamide,

(21) N-(4-fluorobenzyl)-9-{[[(2R)-2-hydroxy-2-pyrazin-2-ylethyl](methyl)amino]methyl}-1-methyl-2,7-dioxo-2,3-dihydro-1H,7H-pyrido[1,2,3-de]quinoxaline-6-carboxamide,

(22) N-(4-chlorobenzyl)-9-{[[(2R)-2-(2-furyl)-2-hydroxyethyl](methyl)amino]methyl}-1-(2-hydroxyethyl)-2,7-dioxo-2,3-dihydro-1H,7H-pyrido[1,2,3-de]quinoxaline-6-carboxamide,

(23) N-(4-fluorobenzyl)-9-{[[(2R)-2-hydroxy-2-thien-2-ylethyl](methyl)amino]methyl}-1-methyl-2,7-dioxo-2,3-dihydro-1H,7H-pyrido[1,2,3-de]quinoxaline-6-carboxamide,

(24) 9-{[[(2S)-2-(1-benzofuran-2-yl)-2-hydroxyethyl](methyl)amino]methyl}1-N-(4-chlorobenzyl)-1-methyl-2,7-dioxo-2,3-dihydro-1H,7H-pyrido[1,2,3-de]quinoxaline-6-carboxamide, or

(25) 9-{[[(2S)-2-(1-benzofuran-2-yl)-2-hydroxyethyl](methyl)amino]methyl}-N-(4-fluorobenzyl)-1-methyl-2,7-dioxo-2,3-dihydro-1H,7H-pyrido[1,2,3-de]quinoxaline-6-carboxamide.

Other examples of the present invention are:

(1) N-(4-chlorobenzyl)-9-{[[(2R)-2-hydroxy-2-(4-hydroxyphenyl)ethyl](methyl)amino]methyl}-1-methyl-2,7-dioxo-2,3-dihydro-1H,7H-pyrido[1,2,3-de]quinoxaline-6-carboxamide,

(2) N-(4-chlorobenzyl)-9-{[[(2S)-2-hydroxy-2-(4-hydroxyphenyl)ethyl](methyl)amino]methyl}-1-methyl-2,7-dioxo-2,3-dihydro-1H,7H-pyrido[1,2,3-de]quinoxaline-6-carboxamide,

(3) N-(4-chlorobenzyl)-9-{[[(2R)-2-hydroxy-2-(5-methyl-2-furyl)ethyl](methyl)amino]methyl}-1-methyl-2,7-dioxo-2,3-dihydro-1H,7H-pyrido[1,2,3-de]quinoxaline-6-carboxamide,

(4) N-(4-chlorobenzyl)-9-{[[(2S)-2-hydroxy-2-(5-methyl-2-furyl)ethyl](methyl)amino]methyl}-1-methyl-2,7-dioxo-2,3-dihydro-1H,7H-pyrido[1,2,3-de]quinoxaline-6-carboxamide,

(5) 9-{[[(2R)-2-(1-benzothien-3-yl)-2-hydroxyethyl](methyl)amino]methyl}-N-(4-chlorobenzyl)-1-methyl-2,7-dioxo-2,3-dihydro-1H,7H-pyrido[1,2,3-de]quinoxaline-6-carboxamide,

(6) 9-{[[(2S)-2-(1-benzothien-3-yl)-2-hydroxyethyl](methyl)amino]methyl}-N-(4-chlorobenzyl)-1-methyl-2,7-dioxo-2,3-dihydro-1H,7H-pyrido[1,2,3-de]quinoxaline-6-carboxamide,

(7) N-(4-chlorobenzyl)-9-{[((2R)-2-hydroxy-2-quinolin-2-ylethyl)(methyl)amino]methyl}-1-methyl-2,7-dioxo-2,3-dihydro-1H,7H-pyrido[1,2,3-de]quinoxaline-6-carboxamide,

(8) N-(4-chlorobenzyl)-9-{[((2S)-2-hydroxy-2-quinolin-2-ylethyl)(methyl)amino]methyl}-1-methyl-2,7-dioxo-2,3-dihydro-1H,7H-pyrido[1,2,3-de]quinoxaline-6-carboxamide

(9) 9-{[[(2R)-2-(1-benzofuran-2-yl)-2-hydroxyethyl](methyl)amino]methyl}-N-(4-fluorobenzyl)-1-methyl-2,7-dioxo-2,3-dihydro-1H,7H-pyrido[1,2,3-de]quinoxaline-6-carboxamide, or

(10) 9-{[[(2S)-2-(1-benzofuran-2-yl)-2-hydroxyethyl](methyl)amino]methyl}-N-(4-fluorobenzyl)-1-methyl-2,7-dioxo-2,3-dihydro-1H,7H-pyrido[1,2,3-de]quinoxaline-6-carboxamide.

As shown in Chart A, the starting material A-0, whose preparation is described in U.S. Pat. No. 6,093,732, is activated with methanesulfonyl chloride and treated with secondary amines to provide compounds of formula A-1, wherein R′ is aryl or heteroaryl. Compounds of formula A-1 are alkylated with short chain alkyl or phenyl bromoacetates to give compounds of formula A-2, wherein R″ is a short chain alkyl or phenyl. Compounds of formula A-2 are reacted with primary amines in suitable solvents (e.g, methanol or THF) to yield amides of formula A-3. Compounds of formula A-3 are reacted with a suitable base (e.g., KOtBu) in the appropriate solvent (e.g., THF) to provide compounds of formula A-4. The secondary amines which will comprise a portion of compounds of formula A-4 can be utilized as a racemic mixture comprising a 1:1 mixture of the R- and S-optical antipodes. Alternatively, secondary amines which will comprise a portion of compounds of formula A-4 can be as optically enriched or single optical antipodes (either the R- or S-antipode dominates or is the sole constituent). The secondary amines afford compounds which are racemates or configured as previously illustrated for compounds of formulas IA and IB as a function of the nature of the amine utilized.

### CHART A

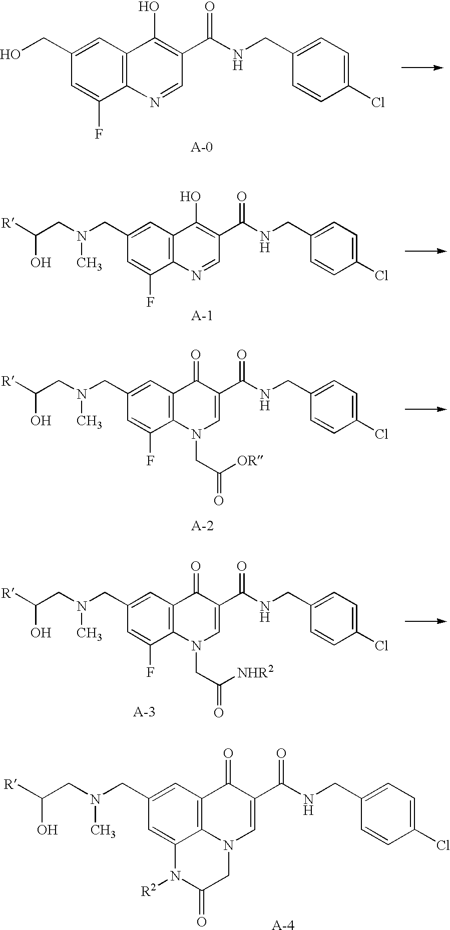

As shown in Chart B, the starting material B-0, whose preparation is described in U.S. Pat. No. 6,093,732, is alkylated with a short chain alkyl or phenyl bromoacetate to give the esters of formula B-1, wherein R⁰ can be a short chain alkyl or phenyl. Compounds of formula B-1 are reacted with various primary amines in suitable solvents (e.g, methanol or THF) to yield amides of formula B-2. Compounds of formula B-2 are reacted with KOtBu in THF to provide compounds of formula B-3. Compounds of formula B-3 are treated with ethyl chlorofomate to give compounds of formula B-4. Compounds of formula B-4 are reacted with secondary amines to yield compounds of formula B-5, wherein R′ is aryl or heteroaryl. The secondary amines which will comprise a portion of compounds of formula B-5 can be utilized as a racemic mixture comprising a 1:1 mixture of the R- and S-optical antipodes. Alternatively, secondary amines which will comprise a portion of compounds of formula B-5 can be as optically enriched or single optical antipodes (either the R- or S-antipode dominates or is the sole constituent). The secondary amines afford compounds which are racemates or configured as previously illustrated for compounds of formulas IA and IB as a function of the nature of the amine utilized.

### CHART B

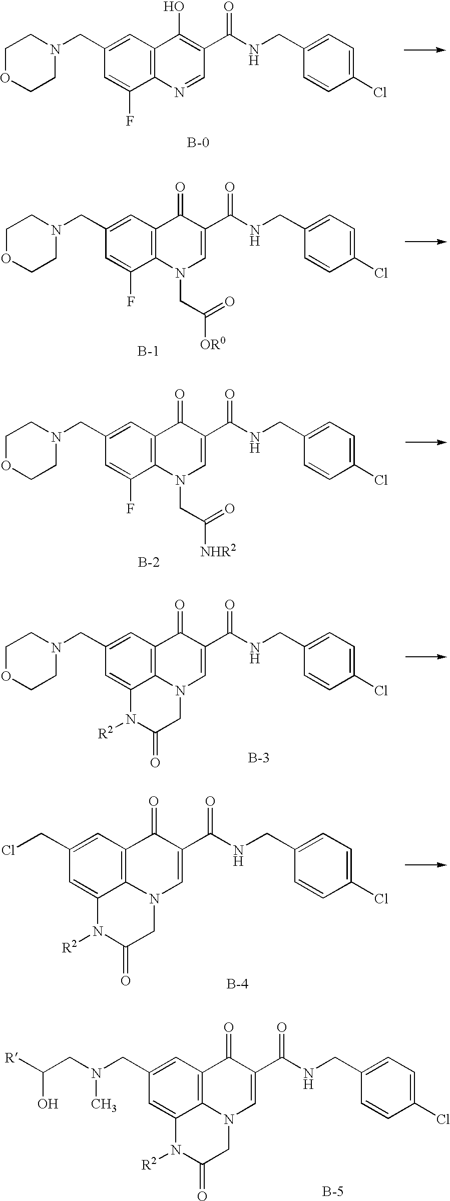

Pharmaceutical Salts

The compound of formula I may be used in its native form or as a salt. In cases where forming a stable nontoxic salt is desired, administration of the compound as a pharmaceutically acceptable salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, ketoglutarate, and glycerophosphate. Suitable inorganic salts may also be formed, including hydrochloride, hydrobromide, sulfate, nitrate, bicarbonate, and carbonate salts.

Pharmaceutically acceptable salts may be obtained using standard procedures well known in the art, for example by reacting a compound of the invention with a suitable acid affording a physiologically acceptable anion.

Routes of Administration

In therapeutic use for treating, or combating, viral infections in a mammal (i.e. human and animals) a compound of the present invention, its pharmaceutical compositions and other antiviral agents can be administered orally, parenterally, topically, rectally, transmucosally, or intestinally.

Parenteral administrations include indirect injections to generate a systemic effect or direct injections to the afflicted area. Examples of parenteral administrations are subcutaneous, intravenous, intramuscular, intradermal, intrathecal, intraocular, intranasal, intravetricular injections or infusions techniques.

Topical administrations include the treatment of infectious areas or organs readily accessibly by local application, such as, for example, eyes, ears including external and middle ear infections, vaginal, open wound, skins including the surface skin and the underneath dermal structures, or other lower intestinal tract. It also includes transdermal delivery to generate a systemic effect.

The rectal administration includes the form of suppositories.

The transmucosal administration includes nasal aerosol or inhalation applications.

The preferred routes of administration are oral and parenteral.

Composition/Formulation

Pharmaceutical compositions of the present invention may be manufactured by processes well known in the art, e.g., by means of conventional mixing, dissolving, granulation, dragee-making, levigating, emulsifying, encapsulating, entrapping, lyophilizing processes or spray drying.

Pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For oral administration, the compounds can be formulated by combining the active compounds with pharmaceutically acceptable carriers well known in the art. Such carriers enable the compounds of the invention to be formulated as tablets, pills, lozenges, dragees, capsules, liquids, solutions, emulsions, gels, syrups, slurries, suspensions and the like, for oral ingestion by a patient. A carrier can be at least one substance which may also function as a diluent, flavoring agent, solubilizer, lubricant, suspending agent, binder, tablet disintegrating agent, and encapsulating agent. Examples of such carriers or excipients include, but are not limited to, magnesium carbonate, magnesium stearate, talc, sugar, lactose, sucrose, pectin, dextrin, mannitol, sorbitol, starches, gelatin, cellulosic materials, low melting wax, cocoa butter or powder, polymers such as polyethylene glycols and other pharmaceutical acceptable materials.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with a filler such as lactose, a binder such as starch, and/or a lubricant such as talc or magnesium stearate and, optionally, stabilizers. In soft capsules, the active compounds may be dissolved or suspended in suitable liquids, such as fatty oils, liquid paraffin, liquid polyethylene glycols, cremophor, capmul, medium or long chain mono-, di- or triglycerides. Stabilizers may be added in these formulations, also.

Liquid form compositions include solutions, suspensions and emulsions. For example, there may be provided solutions of the compounds of this invention dissolved in water and water-propylene glycol and water-polyethylene glycol systems, optionally containing suitable conventional coloring agents, flavoring agents, to stabilizers and thickening agents.

The compounds may also be formulated for parenteral administration, e.g., by injection, bolus injection or continuous infusion. Formulations for parenteral administration may be presented in unit dosage form, e.g., in ampoules or in multi-dose containers, with an added preservative. The compositions may take such forms as suspensions, solutions or emulsions in oily or aqueous vehicles, and may contain formulating materials such as suspending, stabilizing and/or dispersing agents.

For injection, the compounds of the invention may be formulated in aqueous solution, preferably in physiologically compatible buffers or physiological saline buffer. Suitable buffering agents include trisodium orthophosphate, sodium bicarbonate, sodium citrate, N-methylglucamine, L(+)-lysine and L(+)-arginine.

Parenteral administrations also include aqueous solutions of a water soluble form, such as, without limitation, a salt, of the active compound. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances which increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions.

Alternatively, the active ingredient may be in powder form for constitution with a suitable vehicle, e.g., sterile, pyrogen-free water, before use.

For suppository administration, the compounds may also be formulated by mixing the agent with a suitable non-irritating excipient which is solid at room temperature but liquid at rectal temperature and therefore will melt in the rectum to release the drug. Such materials include cocoa butter, beeswax and other glycerides.

For administration by inhalation, compounds of the present invention can be conveniently delivered through an aerosol spray in the form of solution, dry powder, or suspensions. The aerosol may use a pressurized pack or a nebulizer and a suitable propellant. In the case of a pressurized aerosol, the dosage unit may be controlled by providing a valve to deliver a metered amount. Capsules and cartridges of, for example, gelatin for use in an inhaler may be formulated containing a power base such as lactose or starch.

For topical applications, the pharmaceutical composition may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion such as suspensions, emulsion, or cream containing the active components suspended or dissolved in one or more pharmaceutically acceptable carriers. Suitable carriers include, but are not limited to, mineral oil, sorbitan monostearate, polysorbate 60, cetyl esters wax, ceteary alcohol, 2-octyldodecanol, benzyl alcohol and water.

For ophthalmic and otitis uses, the pharmaceutical compositions may be formulated as micronized suspensions in isotonic, pH adjusted sterile saline, or preferably, as solutions in isotonic, pH adjusted sterile saline, either with or without a preservative such as a benzylalkonium chloride. Alternatively, for ophthalmic uses, the pharmaceutical compositions may be formulated in an ointment such as petrolatum.

In addition to the formulations described previously, the compounds may also be formulated as depot preparations. Such long acting formulations may be in the form of implants. A compound of this invention may be formulated for this route of administration with suitable polymers, hydrophobic materials, or as a sparing soluble derivative such as, without limitation, a sparingly soluble salt.

Additionally, the compounds may be delivered using a sustained-release system. Various sustained-release materials have been established and are well known by those skilled in the art. Sustained-release capsules may, depending on their chemical nature, release the compounds for 24 hours or for up to several days.

Dosage

Pharmaceutical compositions suitable for use in the present invention include compositions wherein the active ingredients are contained in an amount sufficient to achieve the intended purpose, i.e., the treatment or prevention of infectious diseases. More specifically, a therapeutically effective amount means an amount of compound effective to prevent, alleviate or ameliorate symptoms of disease or prolong the survival of the subject being treated.

The quantity of active component, that is the compound of this invention, in the pharmaceutical composition and unit dosage form thereof may be varied or adjusted widely depending upon the manner of administration, the potency of the particular compound and the desired concentration. Determination of a therapeutically effective amount is well within the capability of those skilled in the art. Generally, the quantity of active component will range between 0.5% to 90% by weight of the composition.

Generally, an antiviral effective amount of dosage of active component will be in the range of about 0.1 to about 400 mg/kg of body weight/day, more preferably about 1.0 to about 50 mg/kg of body weight/day. It is to be understood that the dosages may vary depending upon the requirements of each subject and the severity of the viral infection being treated. In average, the effective amount of active component is about 200/mg to 800/mg and preferable 600/mg per day.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Also, it is to be understood that the initial dosage administered may be increased beyond the above upper level in order to rapidly achieve the desired plasma concentration. On the other hand, the initial dosage may be smaller than the optimum and the daily dosage may be progressively increased during the course of treatment depending on the particular situation. If desired, the daily dose may also be divided into multiple doses for administration, e.g., two to four times per day.

In cases of local administration or selective uptake, the effective local concentration of the drug may not be related to plasma concentration and other procedures know in the art may be used to determine the desired dosage amount.

Combination Therapy

In combating the infective diseases caused by viruses, the compound of the formula I can be used either individually, or in combination with other antiviral agents that are active against diseases caused by viruses.

Examples of such antiviral agents and the amount, route, and frequency of administration of such active agents are listed in Table 1.

TABLE 1
Active Agents Used in Combination with a Compound of Formula (II)

| | | | | | | Frequency of |
|---|---|---|---|---|---|---|
| Active Agent | Lo Dose | | Hi Dose | | Route | Administration |
| Acyclovir | 100 | mg | 800 | mg | Oral | 2-5 times/day |
| Acyclovir | 1% | ointment | 5% | ointment | Topical | — |
| Penciclovir | 1% | ointment | 5% | ointment | Topical | — |
| Famciclovir | 100 | mg | 500 | mg | Oral | 1-3 times/day |
| Valaciclovir | 250 | mg | 1000 | mg | Oral | 1-3 times/day |
| Ganciclovir | 1 | mg/kg | 5 | mg/kg | IV | 1-2 times/day |
| Ganciclovir | 500 | mg | 1000 | mg | Oral | 2-6 times/day |
| Valganciclovir | 200 | mg/day | 2000 | mg/day | Oral | — |
| Foscarnet | 20 | mg/kg/day | 120 | mg/kg/day | IV | — |
| Cidofovir | 1 | mg/kg/week | 5 | mg/kg/week | IV | — |

The term “Lo Dose” means the recommended lower dosage for the combination therapy of the invention. It may be adjusted even lower depending on the requirements of each subject being treated and the severity of the viral infection. The lowest dosage possible may be 0.1 mg when combined with the compound of formula (II) of the present invention. The term “Hi Dose” means the recommended highest dosage in the combination therapy. It may be changed hereafter according to the US FDA standard. A specific active agent may have more than one recommended dosage range, particularly for different routes of administration.

For the combination therapy, the compound of formula I may be administered concurrently or concomitantly with other antiviral agents. The term “concurrently” means the subject being treated takes one drug within about 5 minutes of taking the other drug. The term “concomitantly” means the subject being treated takes one drug within the same treatment period of taking the other drug. The same treatment period is preferably within twelve hours and up to forty-eight hours.

For the combination therapy, the compound of formula I, and one or more other antiviral agents may be administered in the same physical form or separately, i.e., they may be administered in the same delivery vehicle or in different delivery vehicles.

Specifically, the combination therapy of the present invention is the compound of formula I of the present invention with Acyclovir.

Specifically, the combination therapy of the present invention is the compound of formula I of the present invention with Penciclovir.

Specifically, the combination therapy of the present invention is the compound of formula I of the present invention with Famciclovir.

Specifically, the combination therapy of the present invention is the compound of formula I of the present invention with Valaciclovir.

Specifically, the combination therapy of the present invention is the compound of formula I of the present invention with Ganciclovir.

Specifically, the combination therapy of the present invention is the compound of formula I of the present invention with Valganciclovir.

Specifically, the combination therapy of the present invention is the compound of formula I of the present invention with Foscarnet.

Specifically, the combination therapy of the present invention is the compound of formula I of the present invention with Cidofovir.

### Biological Data

While many of the compounds of the present invention have shown activity against the CMV polymerase, these compounds may be active against the cytomegalovirus by this or other mechanisms of action. Thus, the description below of these compounds' activity against the CMV polymerase is not meant to limit the present invention to a specific mechanism of action.

The compounds of the present invention have shown activity in one or more of the assays described below. All of these assays are indicative of a compound's activity and thus of its use as an anti-viral agent.

The HCMV polymerase assay is performed using a scintillation proximity assay (SPA) as described in several references, such as N.D. Cook, et al., Pharmaceutical Manufacturing International, pages 49-53 (1992); K. Takeuchi, Laboratory Practice, September issue (1992); U.S. Pat. No. 4,568,649 (1986); which are incorporated by reference herein. Reactions are performed in 96-well plates. The assay is conducted in 100 μl volume with 5.4 mM HEPES (pH 7.5), 11.7 MM KCl, 4.5 mM MgCl₂, 0.36 mg/ml BSA, and 90 nM ³H-dTTP. Assays are run with and without CHAPS, (3-[(3-Cholamidopropyl)-dimethylammoniol-1-propane-sulfonate) at a final concentration of 2 mM. HCMV polymerase is diluted in enzyme dilution buffer containing 50% glycerol, 250 mM NaCl, 10 mM HEPES (pH 7.5), 100 μg/ml BSA, and 0.01% sodium azide. The HCMV polymerase, which is expressed in recombinant baculovirus-infected SF-9 cells and purified according to literature procedures, is added at 10% (or 10 μl) of the final reaction volume, i.e., 100 μl. Compounds are diluted in 50% DMSO and 10 μl are added to each well. Control wells contain an equivalent concentration of DMSO. Unless noted otherwise, reactions are initiated via the addition of 6 nM biotinylated poly(dA)-oligo(dT) template/primer to reaction mixtures containing the enzyme, substrate, and compounds of interest. Plates are incubated in a 25° C. or 37° C. H₂O bath and terminated via the addition of 40 μl/reaction of 0.5 M EDTA (pH 8) per well. Reactions are terminated within the time-frame during which substrate incorporation is linear and varied depending upon the enzyme and conditions used, i.e., 30 min. for HCMV polymerase. Ten (10) μl of streptavidin-SPA beads (20 mg/ml in PBS/10% glycerol) are added following termination of the reaction. Plates are incubated 10 min. at 37° C., then equilibrated to room temperature, and counted on a Packard Topcount. Linear regressions are performed and IC₅₀'s are calculated using computer software.

A modified version of the above HCMV polymerase assay is performed as described above, but with the following changes: Compounds are diluted in 100% DMSO until final dilution into assay buffer. In the previous assay, compounds are diluted in 50% DMSO. 4.5 mM dithiotherotol (DTT) is added to the polymerase buffer. Also, a different lot of CMV polymerase is used, which appears to be more active resulting in a more rapid polymerase reaction.

Results of the testing of compounds of the present invention in this assay are shown in Tables 2 below.

All results are listed as Polymerase IC₅₀ (μM) values. In Table 2, the term “--” refers to activity data not determined.

| | CMV | Alpha |
|---|---|---|
| Polymerase | Polymerase | |
| Example No. | IC50 (uM) | IC50 (uM) |
| 1 | 0.220 | — |
| 2 | 0.070 | >5.0 |
| | 0.0930 | — |
| 3 | 0.0086 | >5.0 |
| | 0.0078 | — |
| | 0.0072 | — |
| 4 | 0.570 | — |
| 5 | 0.240 | — |
| 6 | 0.120 | >20.0 |
| | 0.0890 | — |
| 7 | 0.250 | — |
| 8 | 1.10 | — |
| 9 | 0.300 | — |
| 10 | 0.240 | — |
| 11 | 0.110 | >20.0 |
| | 0.0460 | — |
| 12 | 0.130 | 20.0 |
| | 0.0330 | — |
| 13 | 0.150 | >20.0 |
| | 0.0760 | — |
| 14 | 0.190 | — |
| 15 | 0.110 | >20.0 |
| | 0.0860 | — |
| 16 | 0.0480 | >20.0 |
| | 0.0360 | — |
| 17 | 0.870 | — |
| 18 | 0.180 | — |
| 19 | 0.490 | — |
| 20 | 0.410 | — |
| 21 | 0.160 | >10.0 |
| | 0.120 | — |
| 22 | 0.490 | — |
| 23 | 0.420 | — |
| 24 | 1.870 | — |
| 25 | 0.930 | — |
| 26 | 0.880 | — |
| 27 | 2.080 | — |
| 28 | 0.690 | — |
| 29 | 0.810 | — |
| 30 | 1.090 | — |
| 31 | 2.890 | — |
| 32 | 0.580 | — |
| 33 | 0.032 | 4.3900 |
| 34 | 0.0040 | — |
| 35 | 0.180 | — |
| 36 | <0.03 | — |
| 37 | 1.19 | — |

Without further elaboration, it is believed that one skilled in the art can, using the preceding description, practice the present invention to its fullest extent. The foregoing detailed description is given for clearness of understanding only, and no unnecessary limitations should be understood therefrom, as modifications within the scope of the invention may become apparent to those skilled in the art.

### EXAMPLES

### Example 1

### Preparation of N-(4-chlorobenzyl)-9-{[(2-hydroxy-2-phenylethyl) (methyl)amino]methyl}-1-methyl-2,7-dioxo-2,3-dihydro-1H,7H-pyrido[1,2,3-de]quinoxaline-6-carboxamide.

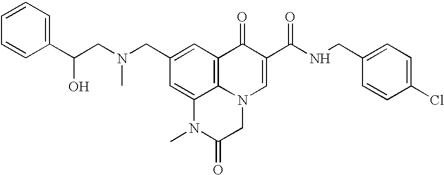

Step 1 Preparation of methyl [3-{[(4-chlorobenzyl)amino]carbonyl}-8-fluoro-6-(morpholin-4-ylmethyl)-4-oxoquinolin-1 (4H)-yl]acetate.

To a flask containing N-(4-chlorobenzyl)-8-fluoro-4-hydroxy-6-(morpholin-4-ylmethyl)quinoline-3-carboxamide (preparation described in U.S. Pat. No. 6,093,732) (10.8 g) is added dimethylformamide (DMF) (100 mL) and potassium carbonate (8.85 g). The resulting suspension is placed under a drying tube and stirred. To the stirring mixture is added methyl bromoacetate (4.8 mL). After stirring overnight, the suspension is diluted with water (250 mL) and filtered. The precipitant is washed with two additional portions of water (2×100 mL). The washed solid is dried under a flow of air and then in a vacuum oven (60° C.) to yield 12.2 g of the title compound as a tan solid. Physical characteristics: ¹H NMR (CDCl₃) δ 2.5, 3.6, 3.7, 3.8, 4.6, 5.1, 7.3, 7.5, 8.2, 8.7, 10.3; MS (ESI+) for m/z 502 (M+H)⁺.

Step 2 Preparation of N-(4-chlorobenzyl)-8-fluoro-1-[2-(methylamino)-2-oxoethyl]-6-(morpholin-4-ylmethyl)-4-oxo-1,4-dihydroquinoline-3-carboxamide.

To a pressure tube containing methyl [3-{[(4-chlorobenzyl)amino]carbonyl}-8-fluoro-6-(morpholin-4-ylmethyl)-4-oxoquinolin-1(4H)-yl]acetate (0.48 g) is added methylamine as a 2 M solution in methanol (12 mL). The mixture is tightly capped, heated to 60° C. and stirred. After 2 days, the suspension is cooled to room temperature and concentrated under reduced pressure. The residue is treated with acetonitrile:methanol (1:1, 50 mL) and heated to reflux. The resulting suspension is cooled to room temperature and placed in a freezer. The solid is collected, washed with diethyl ether and hexanes, dried under a flow of air, and finally in a vacuum oven (60° C.) to yield 0.47 g of the title compound as a white solid. Physical characteristics: ¹H NMR (d₆-DMSO) δ 2.4, 2.7, 3.6, 4.6, 5.5, 7.4, 7.6, 8.1, 8.3, 8.8, 10.3; MS (ESI+) for m/z 501 (M+H)⁺.

Step 3 Preparation of N-(4-chlorobenzyl)-1-methyl-9-(morpholin-4-ylmethyl)-2,7-dioxo-2,3-dihydro-1H,7H-pyrido [1,2,3-de]quinoxaline-6-carboxamide.

To a flame-dried flask under a nitrogen atmosphere is added N-(4-chlorobenzyl)-8-fluoro-1-[2-(methylamino)-2-oxoethyl]-6-(morpholin-4-ylmethyl)-4-oxo-1,4-dihydroquinoline-3-carboxamide (0.10 g) followed by THF (10 mL). The mixture is treated with a solution of potassium tert-butoxide in THF (1 M, 0.20 mL). After 3 hours, the resulting dark red solution is diluted with dichloromethane and partitioned against pH 7 aqueous phosphate buffer. The aqueous layer is extracted with two additional portions of dichloromethane. The combined organic layer is washed brine, dried over sodium sulfate, filtered and concentrated under reduced pressure. The resulting residue is flash column chromatographed on silica eluting with 1% to 5% methanol in dichloromethane. The product containing fractions are combined and concentrated under reduced pressure. The resulting residue is crystallized from acetonitrile:methanol. The solid is collected to yield 0.04 g of the title compound as a white solid. Physical characteristics: m.p. 213-214° C.; analysis found for C₂₅H₂₅ClN₄O₄: C 62.27; H, 5.25; N, 11.60; ¹H NMR (d₆-DMSO) δ 2.4, 3.4, 3.6, 4.6, 5.2, 7.4, 7.9, 8.8, 10.4; MS (ESI+) for m/z 481 (M+H)⁺.

Step 4 Preparation of N-(4-chlorobenzyl)-9-(chloromethyl)-1-methyl-2,7-dioxo-2,3-dihydro-1H,7H-pyrido[1,2,3-de]quinoxaline-6-carboxamide.

To a pressure tube containing N-(4-chlorobenzyl)-1-methyl-9-(morpholin-4-ylmethyl)-2,7-dioxo-2,3-dihydro-1H,7H-pyrido [1,2,3-de]quinoxaline-6-carboxamide (0.10 g) is added chloroform (2 mL) and ethyl chloroformate (0.05 mL). The solution is tightly capped, heated to 60° C. and stirred overnight. The resulting suspension is cooled to room temperature and diluted with diethyl ether. The mixture is filtered and the collected precipitant washed with diethyl ether and hexanes, dried under a flow of air, and finally in a vacuum oven to yield 0.09 g of the title compound as a white solid. Physical characteristics: ¹H NMR (d₆-DMSO) δ 3.4, 4.6, 5.0, 5.2, 7.4, 7.6, 8.0, 8.8, 10.3.

Step 5 Preparation of N-(4-chlorobenzyl)-9-{[(2-hydroxy-2-phenylethyl)(methyl)amino]methyl}-1-methyl-2,7-dioxo-2,3-dihydro-1H,7H-pyrido[1,2,3-de]quinoxaline-6-carboxamide

To a flask containing N-(4-chlorobenzyl)-9-(chloromethyl)-1-methyl-2,7-dioxo-2,3-dihydro-1H,7H-pyrido[1,2,3-de]quinoxaline-6-carboxamide (0.09 g) is added α-(methylaminomethyl)benzyl alcohol (0.05 g), diisopropylethylamine (0.05 mL) and DMF (2 mL). The resulting suspension is stirred at room temperature. After 4 days, the reaction mixture is diluted with ethyl acetate (50 mL), washed with pH 7 phosphate buffer (3×10 mL), brine, dried over sodium sulfate, filtered, and finally concentrated under reduced pressure. The residue is flash column chromatographed on silica eluting with 2% to 4% methanol in dichloromethane. The product containing fractions are combined and concentrated under reduced pressure. The resulting residue is crystallized from ethyl acetate:hexanes to yield 0.04 g of the title compound as a white solid. Physical characteristics: ¹H NMR (d₆-DMSO) δ 2.3, 2.5, 3.3, 3.7, 4.6, 4.8, 5.1, 5.2, 7.2-7.5, 7.8, 8.7, 10.4; MS (ESI+) for m/z 545 (M+H)⁺.

### Example 2

### Preparation of rac N-(4-chlorobenzyl)-9-{([2-hydroxy-2-(4-hydroxyphenyl)ethyl](methyl)amino)methyl}-1-methyl-2,7-dioxo-2,3-dihydro-1H,7H-pyrido[1,2,3-de]quinoxaline-6-carboxamide.

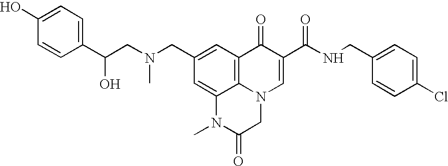

To a flask containing N-(4-chlorobenzyl)-9-(chloromethyl)-1-methyl-2,7-dioxo-2,3-dihydro-1H,7H-pyrido[1,2,3-de]quinoxaline-6-carboxamide (0.43 g) and synephrine (0.27 g) is added DMF (10 mL) and diisopropylethylamine (0.80 mL). After 3 days of stirring at room temperature, the reaction mixture is diluted with ethyl acetate (150 mL) and washed with dilute pH 4 phosphate buffer, dilute pH 7 phosphate buffer, brine, dried over sodium sulfate, filtered, and finally concentrated under reduced pressure. The residue is adsorbed onto silica gel and flash column chromatographed eluting with 2% to 10% methanol in dichloromethane. The product containing fractions are combined and concentrated under reduced pressure. The resulting residue is crystallized from acetonitrile and is then recrystallized from methanol—toluene to yield 0.14 g of the title compound as a white solid. Physical characteristics: Mp 145-148° C.; ¹H NMR (d₆-DMSO) δ 10.4, 9.22, 8.73, 7.79, 7.4, 7.09, 6.67, 5.21, 4.94, 4.7, 4.57, 3.7, 3.3, 2.6-2.4, 2.23; HRMS (ESI+) m/z 561.1881 (M+H)⁺; Anal. Found: C, 64.16; H, 5.12; N, 9.93.

### Example 3

### Preparation of rac 9-{([2-(1-benzofuran-2-yl)-2-hydroxyethyl](methyl)amino)methyl}-N-(4-chlorobenzyl)-1-methyl-2,7-dioxo-2,3-dihydro-1H,7H-pyrido[1,2,3-de]quinoxaline-6-carboxamide.

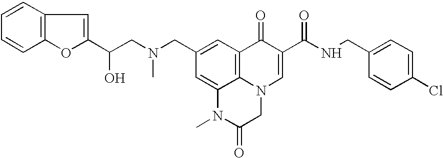

To a flask containing N-(4-chlorobenzyl)-9-(chloromethyl)-1-methyl-2,7-dioxo-2,3-dihydro-1H,7H-pyrido[1,2,3-de]quinoxaline-6-carboxamide (0.21 g) and 1-(1-benzofuran-2-yl)-2-(methylamino)ethanol (0.13 g) is added DMF (5 mL) and diisopropylethylamine (0.5 mL). After 3 days of stirring at room temperature, the reaction mixture is diluted with ethyl acetate (100 mL), washed with dilute pH 4 phosphate buffer (4×10 mL), brine, dried over sodium sulfate, filtered, and finally concentrated under reduced pressure. The residue is flash column chromatographed on silica eluting with 4% to 9% methanol in ethyl acetate. The product containing fractions are combined and concentrated under reduced pressure. The resulting residue is crystallized from toluene to yield 0.22 g of the title compound as an off-white solid. Physical characteristics: Mp 136-139° C.; ¹H NMR (d₆-DMSO) δ 10.4, 8.72, 7.77, 7.54, 7.4, 7.2, 7.12, 6.74, 5.55, 5.17, 4.9, 4.57, 3.7, 3.00, 2.9, 2.5, 2.30; HRMS (ESI+) m/z 585.1899 (M+H)⁺; Anal. Found: C, 65.42; H, 5.19; N, 9.59.

### Example 4

### Preparation of rac N-(4-chlorobenzyl)-9-{([2-(3-furyl)-2-hydroxyethyl](methyl)amino)methyl}-1-methyl-2,7-dioxo-2,3-dihydro-1H,7H-pyrido[1,2,3-de]quinoxaline-6-carboxamide.

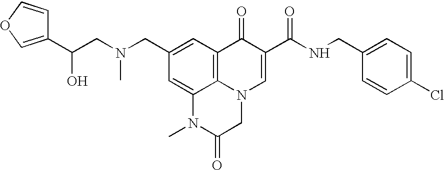

To a flask containing N-(4-chlorobenzyl)-9-(chloromethyl)-1-methyl-2,7-dioxo-2,3-dihydro-1H,7H-pyrido[1,2,3-de]quinoxaline-6-carboxamide (0.21 g) and 1-(3-furyl)-2-(methylamino)ethanol (0.10 g) is added DMF (5 mL) and diusopropylethylamine (0.5 mL). After 3 days of stirring at room temperature, the reaction mixture is diluted with ethyl acetate (100 mL), washed with dilute pH 4 phosphate buffer (4×10 mL), brine, dried over sodium sulfate, filtered, and finally concentrated under reduced pressure. The residue is flash column chromatographed on silica eluting with 4% to 9% methanol in ethyl acetate. The product containing fractions are combined and concentrated under reduced pressure. The resulting residue is crystallized from toluene to yield 0.20 g of the title compound as a white solid. Physical characteristics: Mp 145-148° C.; ¹H NMR (d₆-DMSO) δ 10.4, 8.73, 7.81, 7.6, 7.4, 6.43, 5.22, 5.02, 4.75, 4.57, 3.7, 3.35, 2.7-2.5, 2.24; HRMS (ESI+) m/z 535.1770 (M+H)⁺; Anal. Found: C, 62.63; H, 5.18; N, 10.42.

### Example 5

### Preparation of rac N-(4-chlorobenzyl)-9-{([2-(2-furyl)-2-hydroxyethyl](methyl)amino)methyl}-1-methyl-2,7-dioxo-2,3-dihydro-1H,7H-pyrido[1,2,3-de]quinoxaline-6-carboxamide.

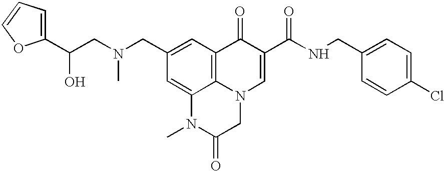

To a flask containing N-(4-chlorobenzyl)-9-(chloromethyl)-1-methyl-2,7-dioxo-2,3-dihydro-1H,7H-pyrido[1,2,3-de]quinoxaline-6-carboxamide (0.21 g) and 1-(2-furyl)-2-(methylamino)ethanol (0.10 g) is added DMF (5 mL) and diisopropylethylamine (0.5 m]L). After 3 days of stirring at room temperature, the reaction mixture is diluted with ethyl acetate (100 mL), washed with dilute pH 4 phosphate buffer (4×10 mL), brine, dried over sodium sulfate, filtered, and finally concentrated under reduced pressure. The residue is flash column chromatographed on silica eluting with 4% to 9% methanol in ethyl acetate. The product containing fractions are combined and concentrated under reduced pressure. The resulting residue is crystallized from toluene to yield 0.21 g of the title compound as a white solid. Physical characteristics: Mp 153-155° C.; ¹H NMR (d₆-DMSO) δ 10.4, 8.73, 7.80, 7.55, 7.4, 7.3, 6.38, 6.28, 5.28, 5.22, 4.77, 4.57, 3.7, 3.34, 2.7, 2.21; HRMS (ESI+) m/z 535.1768 (M+H)⁺;

Anal. Found: C, 62.62; H, 5.05; N, 10.40.

### Example 6

### Preparation of rac N-(4-chlorobenzyl)-9-{([2-hydroxy-2-(5-methyl-2-furyl)ethyl](methyl)amino)methyl}-1-methyl-2,7-dioxo-2,3-dihydro-1H,7H-pyrido[1,2,3-de]quinoxaline-6-carboxamide.

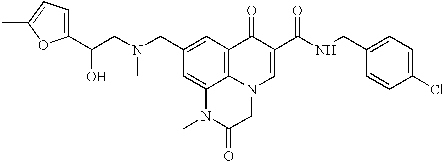

To a flask containing N-(4-chlorobenzyl)-9-(chloromethyl)-1-methyl-2,7-dioxo-2,3-dihydro-1H,7H-pyrido[1,2,3-de]quinoxaline-6-carboxamide (0.14 g) and 2-(methylamino)-1-(5-methyl-2-furyl)ethanol (0.08 g) is added DMF (3 mL) and diisopropylethylamine (0.5 mL). After 4 days of stirring at room temperature, the reaction mixture is diluted with ethyl acetate (100 mL), washed with dilute pH 4 phosphate buffer (4×10 mL), brine, dried over sodium sulfate, filtered, and finally concentrated under reduced pressure. The residue is adsorbed onto silica and flash column chromatographed on silica eluting with 3% to 9% methanol in ethyl acetate. The product containing fractions are combined and concentrated under reduced pressure. The resulting residue is crystallized from toluene to yield 0.14 g of the title compound as a white solid. Physical characteristics: Mp 159-161° C.; ¹H NMR (d₆-DMSO) δ 10.4, 8.73, 7.80, 7.4, 6.12, 5.96, 5.22, 5.15, 4.7, 4.57, 3.7, 3.35, 2.70, 2.22, 2.18; HRMS (ESI+) m/z 549.1920 (M+H)⁺; Anal. Found: C, 63.32; H, 5.44; N, 10.08.

### Example 7

### Preparation of rac N-(4-chlorobenzyl)-9-{([2-(2,5-dimethyl-3-furyl)-2-hydroxyethyl](methyl)amino)methyl}-1-methyl-2,7-dioxo-2,3-dihydro-1H,7H-pyrido[1,2,3-de]quinoxaline-6-carboxamide.

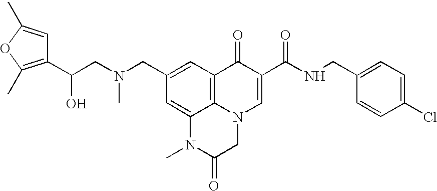

To a flask containing N-(4-chlorobenzyl)-9-(chloromethyl)-1-methyl-2,7-dioxo-2,3-dihydro-1H,7H-pyrido[1,2,3-de]quinoxaline-6-carboxamide (0.14 g) and 1-(2,5-dimethyl-3-furyl)-2-(methylamino)ethanol (0.08 g) is added DMF (3 mL) and diusopropylethylamine (0.5 mL). After 4 days of stirring at room temperature, the reaction mixture is diluted with ethyl acetate (100 mL), washed with dilute pH 4 phosphate buffer (4×10 mL), brine, dried over sodium sulfate, filtered, and finally concentrated under reduced pressure. The residue is adsorbed onto silica and flash column chromatographed on silica eluting with 3% to 9% methanol in ethyl acetate. The product containing fractions are combined and concentrated under reduced pressure. The resulting residue is crystallized from toluene to yield 0.13 g of the title compound as a white solid. Physical characteristics: Mp 157-160° C.; ¹H NMR (d₆-DMSO) δ 10.4, 8.73, 7.80, 7.4, 5.85, 5.22, 4.75, 4.57, 3.7, 3.35, 2.7-2.4, 2.22, 2.16, 2.13; HRMS (ESI+) m/z 563.2039 (M+H)⁺.

### Example 8

### Preparation of rac N-(4-chlorobenzyl)-9-{([2-hydroxy-2-(6-methylpyridin-2-yl)ethyl](methyl)amino)methyl}-1-methyl-2,7-dioxo-2,3-dihydro-1H,7H-pyrido [1,2,3-de]quinoxaline-6-carboxamide.

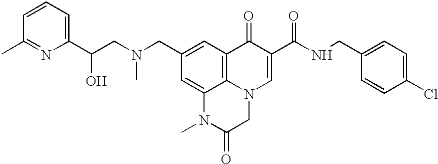

To a flask containing N-(4-chlorobenzyl)-9-(chloromethyl)-1-methyl-2,7-dioxo-2,3-dihydro-1H,7H-pyrido[1,2,3-de]quinoxaline-6-carboxamide (0.14 g) is added DMF (3 mL) followed by 2-(methylamino)-1-(6-methylpyridin-2-yl)ethanol (0.09 g) and diusopropylethylamine (0.5 mL). After 4 days of stirring at room temperature, the reaction mixture is diluted with ethyl acetate (100 mL), washed with dilute pH 4 phosphate buffer (3×10 mL), brine, dried over sodium sulfate, filtered, and finally concentrated under reduced pressure. The residue is flash column chromatographed on silica eluting with 2% to 6% methanol in dichloromethane. The product containing fractions are combined and concentrated under reduced pressure. The resulting residue is crystallized from toluene to yield 0.15 g of the title compound as a white solid. Physical characteristics: Mp 156-158° C.; ¹H NMR (d₆-DMSO) δ 10.4, 8.73, 7.79, 7.63, 7.4, 7.3, 7.1, 5.3, 5.22, 4.8, 4.56, 3.7, 3.35, 2.8-2.6, 2.39, 2.27; HRMS (ESI+) m/z 560.2041 (M+H)⁺; Anal. Found: C, 64.08; H, 5.52; N, 12.40.

### Example 9

### Preparation of N-(4-chlorobenzyl)-9-{[[(2R)-2-hydroxy-2-pyridin-2-ylethyl](methyl)amino]methyl}-1-methyl-2,7-dioxo-2,3-dihydro-1H,7H-pyrido[1,2,3-de]quinoxaline-6-carboxamide.

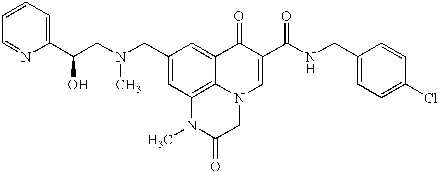

A 25 mL 1-neck 14/20 round bottom flask is charged with N-(4-chlorobenzyl)-9-(chloromethyl)-1-methyl-2,7-dioxo-2,3-dihydro-1H,7H-pyrido[1,2,3-de]quinoxaline-6-carboxamide (0.25 g) and R-2-(1-hydroxy-2-N-methylamino-ethyl)-pyridine (0.177 g). To this mixture is added DMF (5 mL), (i-Pr)₂NEt (0.15 g), and dried, 4 Å powedered molecular sieves (1.16 g). The mixture is allowed to stir at room temperature for 16 hours. The mixture is diluted with CH₂Cl₂ (50 mL), filtered through a pad of Celite® on a coarse scintered glass funnel and the filter cake is rinsed with CH₂Cl₂ (50 mL). The bulk of the solvent is removed on a rotary evaporator and the redisual DMF is removed at high vacuum to a yellow oil. Purification on a 40S Biotage column [wet CH₂Cl₂, eluted CH₂Cl₂, CH₂Cl₂/MeOH (97.5:2.5), CH₂Cl₂/MeOH (95:5), CH₂Cl₂/MeOH (92.5:7.5), CH₂Cl₂/MeOH (90:10) provides 0.301 g of N-(4-chlorobenzyl)-9-{[[(2R)-2-hydroxy-2-pyridin-3-ylethyl](methyl)amino]methyl}-1-methyl-2,7-dioxo-2,3-dihydro-1H,7H-pyrido[1,2,3-de]quinoxaline-6-carboxamide as an ivory solid. Physical characteristics: mp: 125-127° C.; ¹H-NMR (400 MHz, CDCl₃): δ 10.28, 8.68, 8.53, 8.00, 7.70, 7.46, 7.40, 7.31, 7.21, 5.02, 4.94, 4.64, 3.93, 3.73, 3.55, 2.88, 2.74, 2.42; ¹³C-NMR (100 MHz, CDCl₃): δ 182.21, 173.84, 169.88, 162.18, 158.24, 146.58, 142.92, 135.07, 134.71, 130.84, 128.75, 126.89, 126.64, 125.16, 124.70, 118.72, 118.42, 115.56, 111.82, 68.23, 61.70, 59.97, 50.39, 40.59, 40.31, 27.12.; Specific Rotation [α]^{D}₂₅ 51 (c 0.41, chloroform).

### Example 10

### Preparation of rac N-(4-chlorobenzyl)-9-{([2-hydroxy-2-(4-hydroxy-3-methoxyphenyl)ethyl](methyl)amino)methyl}-1-methyl-2,7-dioxo-2,3-dihydro-1H,7H-pyrido[1,2,3-de]quinoxaline-6-carboxamide.

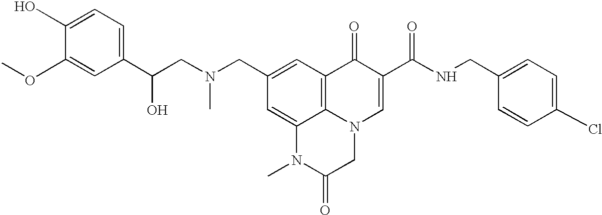

To a flask containing N-(4-chlorobenzyl)-9-(chloromethyl)-1-methyl-2,7-dioxo-2,3-dihydro-1H,7H-pyrido[1,2,3-de]quinoxaline-6-carboxamide (0.14 g) and metanephrine hydrochloride (0.12 g) is added DMF (3 mL) and diusopropylethylamine (1.0 mL). After 7 days of stirring at room temperature, the reaction mixture is diluted with ethyl acetate (100 mL) and washed with dilute pH 7 phosphate buffer (3×). The combined aqueous layers are back extracted with ethyl acetate. The combined organic layers are washed with brine, dried over sodium sulfate, filtered, and finally concentrated under reduced pressure. The residue is precipitated from methanol—toluene to yield 0.12 g of the title compound as an off-white solid. Physical characteristics: Mp 133-136° C.; ¹H NMR (d₆-DMSO) δ 10.4, 8.74, 7.8, 7.4, 7.3, 6.8, 6.7, 5.21, 5.0, 4.7, 4.6, 3.6, 3.3, 2.6, 2.26; HRMS (ESI+) m/z 591.2006 (M+H)⁺; Anal. Found: C, 62.73; H, 5.36; N, 9.32.

### Example 11

### Preparation of N-(4-chlorobenzyl)-9-{[[(2R)-2-(2-furyl)-2-hydroxyethyl](methyl)amino]methyl}-1-methyl-2,7-dioxo-2,3-dihydro-1H,7H-pyrido[1,2,3-de]quinoxaline-6-carboxamide.

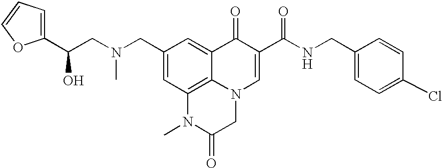

As described in the general procedure outlined in Example 9, a stirring solution of N-methyl R-1-(2-furyl)-2-aminoethanol (0.16 g), powdered 4 Å sieves (1.16 g), iPr₂NEt (0.2 mL) and N-(4-chlorobenzyl)-9-(chloromethyl)-1-methyl-2,7-dioxo-2,3-dihydro-1H,7H-pyrido[1,2,3-de]quinoxaline-6-carboxamide (0.25 g) in DMF (5 mL) affords a crude a yellow oil. The crude material is chromatographed on a 40 g Biotage column (packed with CH₂Cl₂; eluted with CH₂Cl₂, then 5% MeOH/CH₂Cl₂ to afford N-(4-chlorobenzyl)-9-{[[(2R)-2-(2-furyl)-2-hydroxyethyl](methyl)amino]methyl}-1-methyl-2,7-dioxo-2,3-dihydro-1H,7H-pyrido[1,2,3-de]quinoxaline-6-carboxamide as a white solid (0.16 g). Physical characteristics: Mp119-120° C.; ¹H-NMR (300 MHz, CDCl₃): δ 10.28, 8.65, 8.00, 7.22-7.50, 6.34, 6.29, 5.00, 4.85, 4.65, 3.88, 3.72, 3.53, 3.48, 3.00, 2.70, 2.40; HRMS (FAB) found 535.1733 for C₂₈H₂₇CLN₄O₅+H+; Specific Rotation [α]²⁵_{D}=23 (c 0.98, chloroform).

### Example 12

### Preparation of N-(4-chlorobenzyl)-9-{[[(2S)-2-hydroxy-2-phenylethyl](methyl)amino]methyl}-1-methyl-2,7-dioxo-2,3-dihydro-1H,7H-pyrido[1,2,3-de]quinoxaline-6-carboxamide.

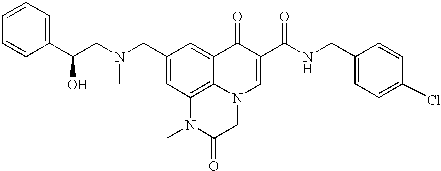

As described in the general procedure outlined in Example 9, powdered 4 Å sieves (1.8 g), iPr₂NEt (0.2 mL) and N-(4-chlorobenzyl)-9-(chloromethyl)-1-methyl-2,7-dioxo-2,3-dihydro-1H,7H-pyrido[1,2,3-de]quinoxaline-6-carboxamide (0.40 g) and S-2-(1-hydroxy-2-N-methylamino-ethyl)-benzene (0.25 g) in DMF (10 mL) affords 0.30 g of N-(4-chlorobenzyl)-9-{[[(2S)-2-hydroxy-2-phenylethyl](methyl)amino]methyl}-1-methyl-2,7-dioxo-2,3-dihydro-1H,7H-pyrido[1,2,3-de]quinoxaline-6-carboxamide as a pale yellow oil. Physical characteristics: ¹H-NMR (300 MHz, DMSO-d₆): δ 10.35, 8.73, 7.95, 7.13-7.42, 5.21, 5.16, 4.76, 4.56, 3.69, 3.30-3.40, 2.63, 2.49, 2.25; HRMS (FAB) found 545.1963 for C₃₀H₂₉CLN₄O₄+H₁; Specific Rotation [α]²⁵_{D}=11 (c 1.00, DMSO).

### Example 13

### Preparation of N-(4-chlorobenzyl)-9-{[[(2S)-2-hydroxy-2-pyridin-3-ylethyl](methyl)amino]methyl}-1-methyl-2,7-dioxo-2,3-dihydro-1H,7H-pyrido[1,2,3-de]quinoxaline-6-carboxamide.

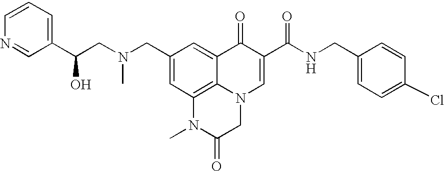

As described in the general procedure outlined in Example 9, 0.40 g of N-(4-chlorobenzyl)-9-(chloromethyl)-1-methyl-2,7-dioxo-2,3-dihydro-1H,7H-pyrido[1,2,3-de]quinoxaline-6-carboxamide, 0.28 g of S-3-(1-hydroxy-2-N-methylamino-ethyl)-pyridine, 0.30 mL of i-Pr₂NEt, and 1.8 g of 4 Å sieves in DMF (10 mL) affords 0.43 g of N-(4-chlorobenzyl)-9-{[[(2S)-2-hydroxy-2-pyridin-3-ylethyl](methyl)amino]methyl}-1-methyl-2,7-dioxo-2,3-dihydro-1H,7H-pyrido[1,2,3-de]quinoxaline-6-carboxamide as an ivory solid. Physical characteristics: mp: 131-135° C.; ¹H-NMR (300 MHz, DMSO-d₆): δ 10.35, 8.72, 8.52, 8.41, 7.75, 7.68, 7.25-7.47, 7.23, 5.37, 5.29, 4.81, 4.56, 3.71, 3.62, 3.29, 2.67, 2.57, 2.25; HRMS (FAB) found 546.1910 for C₂₉H₂₈CLN₅O₄+H₁; Specific Rotation [α]²⁵_{D}=7 (c 1.03, DMSO).

### Example 14

### N-(4-chlorobenzyl)-9-{[[(2S)-2-hydroxy-2-thien-3-ylethyl](methyl)amino]methyl}-1-methyl-2,7-dioxo-2,3-dihydro-1H,7H-pyrido[1,2,3-de]quinoxaline-6-carboxamide.

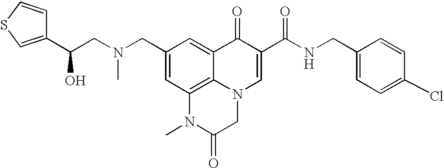

As described in the general procedure outlined in Example 9, 0.40 g of N-(4-chlorobenzyl)-9-(chloromethyl)-1-methyl-2,7-dioxo-2,3-dihydro-1H,7H-pyrido[1,2,3-de]quinoxaline-6-carboxamide, 0.28 g of S-3-(1-hydroxy-2-N-methylamino-ethyl)-thiophene, 0.30 mL of i-Pr₂NEt, and 1.8 g of 4 Å sieves in DMF (10 mL) affords 0.48 g of N-(4-chlorobenzyl)-9-{[[(2S)-2-hydroxy-2-thien-3-ylethyl](methyl) amino]methyl}-1-methyl-2,7-dioxo-2,3-dihydro-1H,7H-pyrido[1,2,3-de]quinoxaline-6-carboxamide as an ivory solid. Physical characteristics: ¹H-NMR (300 MHz, DMSO-d₆): δ 10.35, 8.72, 7.79, 7.25-7.46, 7.06, 5.21, 5.15, 4.85, 4.56, 3.69, 3.37, 3.34, 2.64, 2.24; mp 137-139° C.; HRMS (FAB) found 551.1530 for C₂₈H₂₇CLN₄O₄S+H₁; Specific Rotation [α]²⁵_{D=}40 (c 1.01, chloroform).

### Example 15

### Preparation of rac 9-{([2-(1-benzothien-3-yl)-2-hydroxyethyl](methyl)amino)methyl}-N-(4-chlorobenzyl)-1-methyl-2,7-dioxo-2,3-dihydro-1H,7H-pyrido[1,2,3-de]quinoxaline-6-carboxamide.

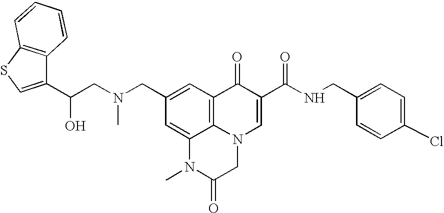

To a flask containing N-(4-chlorobenzyl)-9-(chloromethyl)-1-methyl-2,7-dioxo-2,3-dihydro-1H,7H-pyrido[1,2,3-de]quinoxaline-6-carboxamide (0.14 g) and 1-(1-benzothien-3-yl)-2-(methylamino)ethanol (0.10 g) is added DMF (3 mL) and diisopropylethylamine (0.5 mL). After 4 days of stirring at room temperature, the reaction mixture is diluted with ethyl acetate (100 mL), washed with dilute pH 4 phosphate buffer (4×10 mL), brine, dried over sodium sulfate, filtered, and finally concentrated under reduced pressure. The residue is adsorbed onto silica and flash column chromatographed on silica eluting with 3% to 9% methanol in ethyl acetate. The product containing fractions are combined and concentrated under reduced pressure. The resulting residue is crystallized from toluene and then recrystallized from acetonitrile-methanol to yield 0.12 g of the title compound as a white solid. Physical characteristics: Mp 147-149° C.; ¹H NMR (d₆-DMSO) δ 10.4, 7.98, 7.77, 7.68, 7.54, 7.4, 7.3-7.1, 5.35, 5.21, 5.1, 4.9, 4.57, 3.7, 3.22, 2.9-2.6, 2.37; HRMS (ESI+) m/z 601.1657(M+H)⁺; Anal. Found: C, 63.83; H, 4.93; N, 9.30.

### Example 16

### Preparation of rac N-(4-chlorobenzyl)-9-{([2-hydroxy-2-quinolin-2-ylethyl](methyl)amino)methyl 1-1-methyl-2,7-dioxo-2,3-dihydro-1H,7H-pyrido[1,2,3-de]quinoxaline-6-carboxamide.

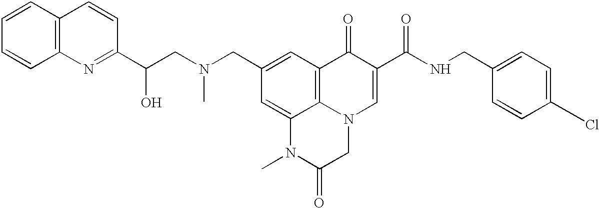

To a flask containing N-(4-chlorobenzyl)-9-(chloromethyl)-1-methyl-2,7-dioxo-2,3-dihydro-1H,7H-pyrido[1,2,3-de]quinoxaline-6-carboxamide (0.14 g) is added DMF (3 mL) followed by 2-(methylamino)-1-quinolin-2-ylethanol (0.10 g) and diisopropylethylamine (0.5 mL). After 6 days of stirring at room temperature, the reaction mixture is diluted with ethyl acetate (90 mL) and washed with dilute pH 4 phosphate buffer (3×10 mL). The combined aqueous layers are back extracted with ethyl acetate and dichloromethane. The combined organic layers are washed with brine, dried over sodium sulfate, filtered, and finally concentrated under reduced pressure. The residue is flash column chromatographed on silica eluting with 3% to 7.5% methanol in dichloromethane. The product containing fractions are combined and concentrated under reduced pressure. The resulting residue is crystallized from toluene to yield 0.11 g of the title compound as a white solid. Physical characteristics: Mp 173-177° C.; ¹H NMR (d₆-DMSO) δ 10.4, 8.71, 8.28, 7.91, 7.85, 7.71, 7.66, 7.58, 7.55, 7.4, 7.04, 5.55, 5.16, 4.9, 4.57, 3.7, 3.01, 2.9-2.7, 2.31; HRMS (ESI+) m/z 596.2068 (M+H)⁺; Anal. Found: C, 66.41; H, 5.17; N, 11.74.

### Example 17

### Preparation of rac N-(4-chlorobenzyl)-9-{[(2-hydroxy-3-phenylpropyl)(methyl)amino]methyl}-1-methyl-2,7-dioxo-2,3-dihydro-1H,7H-pyrido [1,2,3-de]quinoxaline-6-carboxamide.

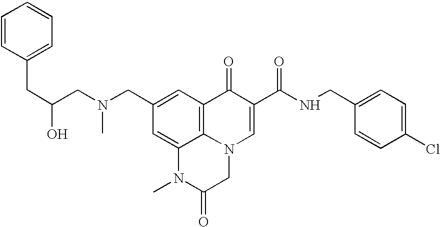

To a flask containing N-(4-chlorobenzyl)-9-(chloromethyl)-1-methyl-2,7-dioxo-2,3-dihydro-1H,7H-pyrido[1,2,3-de]quinoxaline-6-carboxamide (0.14 g) and 1-(methylamino)-3-phenylpropan-2-ol (0.08 g) is added DMF (3 mL) and diisopropylethylamine (0.5 mL). After 7 days of stirring at room temperature, the reaction mixture is diluted with ethyl acetate (60 mL) and washed with dilute pH 4 phosphate buffer (3×). The combined aqueous layers are back extracted with ethyl acetate. The combined organic layers are washed with brine, dried over sodium sulfate, filtered, and finally concentrated under reduced pressure. The residue is crystallized from acetonitrile to yield 0.14 g of the title compound as an off-white solid. Physical characteristics: mp 121-127° C.; ¹H NMR (d₆-DMSO) δ 10.4, 8.74, 7.85, 7.46, 7.4, 7.3-7.1, 5.23, 4.6, 3.9, 3.66, 3.3, 2.8, 2.5, 2.34, 2.22; HRMS (ESI+) m/z 559.2131 (M+H)⁺; Anal. Found: C, 66.44; H, 5.80; N, 9.83.

### Example 18

### Preparatio of rac N-(4-chlorobenzyl)-9-{([2-hydroxy-2-(3-methoxyphenyl)ethyl](methyl)amino)methyl}-1-methyl-2,7-dioxo-2,3-dihydro-1H,7H-pyrido[1,2,3-de]quinoxaline-6-carboxamide.

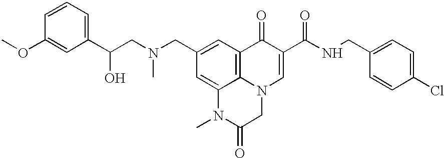

To a flask containing N-(4-chlorobenzyl)-9-(chloromethyl)-1-methyl-2,7-dioxo-2,3-dihydro-1H,7H-pyrido[1,2,3-de]quinoxaline-6-carboxamide (0.14 g) and 1-(3-methoxyphenyl)-2-(methylamino)ethanol (0.09 g) is added DMF (3 mL) and diisopropylethylamine (0.5 mL). After 7 days of stirring at room temperature, the reaction mixture is diluted with ethyl acetate (60 mL), washed with dilute pH 4 phosphate buffer (4×), brine, dried over sodium sulfate, filtered, and finally concentrated under reduced pressure. The residue is crystallized from acetonitrile. The crystals are adsorbed onto silica and flash column chromatographed on silica eluting with 2% to 6% methanol in dichloromethane. The product containing fractions are combined and concentrated under reduced pressure. The resulting residue is crystallized from acetonitrile to yield 0.06 g of the title compound as a white solid. Physical characteristics: Mp 154-156° C.; ¹H NMR (d₆-DMSO) δ 10.4, 8.74, 7.80, 7.4, 7.2, 6.9, 6.8, 5.22, 5.15, 4.75, 4.57, 3.7, 3.3, 2.6, 2.26; HRMS (ESI+) m/z 575.2068 (M+H)⁺; Anal. Found: C, 64.49; H, 5.42; N, 9.69.

### Example 19

### Preparation of rac N-(4-chlorobenzyl)-9-{([2-hydroxy-2-pyrimidin-2-ylethyl](methyl)amino)methyl}-1-methyl-2,7-dioxo-2,3-dihydro-1H,7H-pyrido [1,2,3-de]quinoxaline-6-carboxamide.

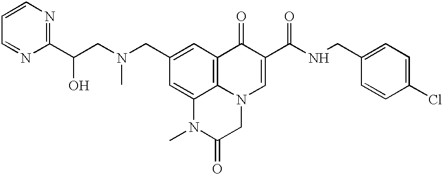

To a flask containing N-(4-chlorobenzyl)-9-(chloromethyl)-1-methyl-2,7-dioxo-2,3-dihydro-1H,7H-pyrido[1,2,3-de]quinoxaline-6-carboxamide (0.27 g) and 2-(methylamino)-1-pyrimidin-2-ylethanol (0.15 g) is added DMF (5 mL) and diisopropylethylamine (1.0 mL). After 4 days of stirring at room temperature, the reaction mixture is diluted with ethyl acetate (100 mL), washed with dilute pH 4 phosphate buffer (2×50 mL), brine, dried over sodium sulfate, filtered, and finally concentrated under reduced pressure. The residue is flash column chromatographed on silica eluting with 2% to 6% methanol in dichloromethane. The product containing fractions are combined and concentrated under reduced pressure. The resulting residue is crystallized from acetonitrile to yield 0.27 g of the title compound as a white solid. Physical characteristics: Mp 168-170 ° C.; ¹H NMR (d₆-DMSO) δ 10.4, 8.77, 8.75, 8.72, 7.71, 7.4, 7.20, 5.32, 5.21, 4.85, 4.57, 3.7, 3.3, 2.9, 2.7, 2.21; HRMS (ESI+) m/z 547.1853 (M+H)⁺; Anal. Found: C, 61.16; H, 4.96; N, 15.30.

### Example 20

### Preparation of rac N-(4-chlorobenzyl)-9-{([2-(5-cyanothein-2-yl)-2-hydroxyethyl](methyl)amino)methyl}-1-methyl-2,7-dioxo-2,3-dihydro-1H,7H-pyrido[1,2,3-de]quinoxaline-6-carboxamide.

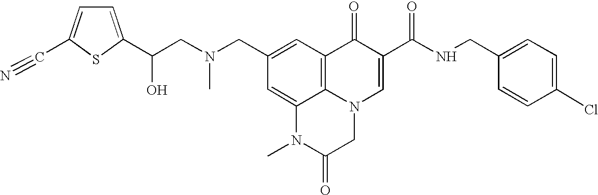

To a flask containing N-(4-chlorobenzyl)-9-(chloromethyl)-1-methyl-2,7-dioxo-2,3-dihydro-1H,7H-pyrido[1,2,3-de]quinoxaline-6-carboxamide (0.27 g) and 5-[1-hydroxy-2-(methylamino)ethyl]thiophene-2-carbonitrile (0.18 g) is added DMF (5 mL) and diisopropylethylamine (1.0 mL). After 4 days of stirring at room temperature, the reaction mixture is diluted with ethyl acetate (100 mL), washed with dilute pH 4 phosphate buffer (2×50 mL), brine, dried over sodium sulfate, filtered, and finally concentrated under reduced pressure. The residue is flash column chromatographed on silica eluting with 2% to 4% methanol in dichloromethane. The product containing fractions are combined and concentrated under reduced pressure. The resulting residue is crystallized from acetonitrile-toluene-methanol to yield 0.23 g of the title compound as a white solid. Physical characteristics: Mp 122-126° C.; ¹H NMR (d₆-DMSO) δ 10.4, 8.73, 7.81, 7.4, 7.14, 6.08, 5.21, 5.06, 4.56, 3.7, 3.35, 2.7, 2.26; HRMS (ESI+) m/z 576.1486 (M+H)⁺; % Water (KF): 0.53; Anal. Found: C, 59.90; H, 4.65; N, 12.07.

### Example 21

### Preparation of N-(4-chlorobenzyl)-9-{[[(2R)-2-hydroxy-2-pyrazin-2-ylethyl](methyl)amino]methyl}-1-methyl-2,7-dioxo-2,3-dihydro-1H,7H-pyrido[1,2,3-de]quinoxaline-6-carboxamide.

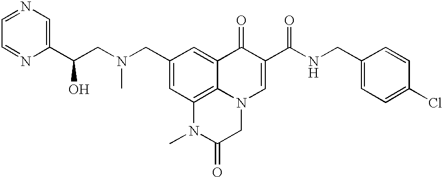

As described in the general procedure outlined in Example 9, 0.32 g of N-(4-chlorobenzyl)-9-(chloromethyl)-1-methyl-2,7-dioxo-2,3-dihydro-1H,7H-pyrido[1,2,3-de]quinoxaline-6-carboxamide, 0.23 g of R-2-(1-hydroxy-2-N-methylamino-ethyl)-pyrazine, 0.25 mL of i-Pr₂NEt, and 0.5 g of 4 Å sieves in DMF (5 mL) affords 0.31 g of N-(4-chlorobenzyl)-9-{[[(2R)-2-hydroxy-2-pyrazin-2-ylethyl](methyl) amino]methyl}-1-methyl-2,7-dioxo-2,3-dihydro-1H,7H-pyrido [1,2,3-de]quinoxaline-6-carboxamide as an ivory solid. Physical characteristics: mp 97-98° C.; ¹H-NMR (400 MHz, DMSO-d₆): δ 10.37, 8.71, 8.51, 7.72, 7.32-7.43, 7.21, 5.62, 5.21, 4.88, 4.56, 3.67, 3.30, 2.83, 2.74, 2.24; HRMS (FAB) found 547.1880 for C₂₈H₂₇CLN₆O₄+H₁; Specific Rotation [α]25D=23 (c 0.91, DMSO).

### Example 22

### Preparation of N-(4-chlorobenzyl)-9-{[[(2R)-2-hydroxy-2-(1,3-thiazol-2-yl)ethyl](methyl)amino]methyl}-1-methyl-2,7-dioxo-2,3-dihydro-1H,7H-pyrido[1,2,3-de]quinoxaline-6-carboxamide.

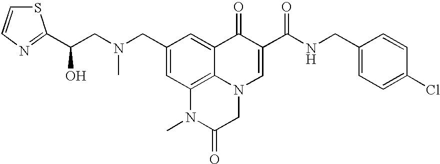

As described in the general procedure outlined in Example 9, 0.46 g of N-(4-chlorobenzyl)-9-(chloromethyl)-1-methyl-2,7-dioxo-2,3-dihydro-1H,7H-pyrido[1,2,3-de]quinoxaline-6-carboxamide, 0.34 g of R-2-(1-hydroxy-2-N-methylamino-ethyl)-thiazole, 0.37 mL of i-Pr₂NEt, and 0.5 g of 4 Å sieves in DMF (5 mL) affords 0.27 g of N-(4-chlorobenzyl)-9-{[[(2R)-2-hydroxy-2-(1,3-thiazol-2-yl)ethyl](methyl)amino]methyl}-1-methyl-2,7-dioxo-2,3-dihydro-1H,7H-pyrido[1,2,3-de]quinoxaline-6-carboxamide as an ivory solid. Physical characteristics: mp125-130° C.; ¹H-NMR (400 MHz, DMSO-d₆): δ 10.36, 8.72, 7.80, 7.72, 7.62, 7-30-7.42, 6.18, 5.76, 5.21, 5.07, 4.56, 3.73, 3.36, 2.85, 2.74, 2.26; HRMS (FAB) found 552.1469 for C₂₇H₂₆CLN₅O₄S+H₁; Specific Rotation [α]²⁵_{D}=12 (c 0.96, DMSO).

### Example 23

### Preparation of N-(4-fluorobenzyl)-9-{[[(2R)-2-(2-furyl)-2-hydroxyethyl](methyl)amino]methyl}-1-methyl-2,7-dioxo-2,3-dihydro-1H,7H-pyrido[1,2,3-de]quinoxaline-6-carboxamide.

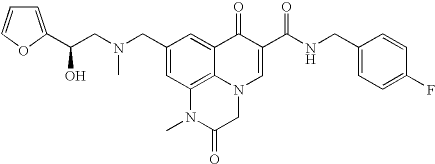

As described in the general procedure outlined in Example 9, 0.25 g of N-(4-fluorobenzyl)-9-(chloromethyl)-1-methyl-2,7-dioxo-2,3-dihydro-1H,7H-pyrido[1,2,3-de]quinoxaline-6-carboxamide, 0.17 g of N-Methyl R-1-(2-furyl)-2-aminoethanol, 0.21 mL of i-Pr₂NEt, and 0.3 g of 4 Å sieves in DMF (5 mL) affords 0.18 g of N-(4-fluorobenzyl)-9-{[[(2R)-2-(2-furyl)-2-hydroxyethyl](methyl)amino]methyl}-1-methyl-2,7-dioxo-2,3-dihydro-1H,7H-pyrido[1,2,3-de]quinoxaline-6-carboxamide as an ivory solid. Physical characteristics: mp 150-152° C.; ¹H-NMR (400 MHz, DMSO-d₆): δ 10.33, 8.72, 7.78, 7.44, 7.39, 7.26, 7.16, 6.33, 6.27, 5.27, 5.21, 4.75, 4.55, 3.66, 3.37, 2.71, 2.23; HRMS (FAB) found 519.2043 for C₂₈H₂₇FN₄O₅+H₁; Specific Rotation [α]²⁵_{D}=−2 (c 0.97, DMSO).

### Example 24

### Preparation of N-(4-fluorobenzyl)-9-{[[(2R)-2-hydroxy-2-pyridin-2-ylethyl](methyl)amino]methyl}-1-methyl-2,7-dioxo-2,3-dihydro-1H,7H-pyrido[1,2,3-de]quinoxaline-6-carboxamide.

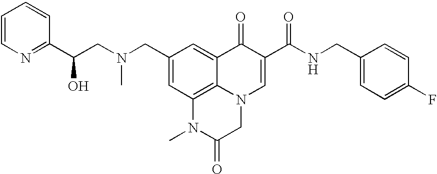

As described in the general procedure outlined in Example 9, 0.25 g of N-(4-fluorobenzyl)-9-(chloromethyl)-1-methyl-2,7-dioxo-2,3-dihydro-1H,7H-pyrido[1,2,3-de]quinoxaline-6-carboxamide, 0.18 g of N-methyl R-2-(1-hydroxy-2-N-methylamino-ethyl)-pyridine, 0.21 mL of i-Pr₂NEt, and 0.30 g of 4 Å sieves in DMF (5 mL) affords 0.16 g of N-(4-fluorobenzyl)-9-{[[(2R)-2-hydroxy-2-pyridin-2-ylethyl](methyl)amino]methyl}-1-methyl-2,7-dioxo-2,3-dihydro-1H,7H-pyrido[1,2,3-de]quinoxaline-6-carboxamide as a pale yellow solid. Physical characteristics: mp 121-123; ¹H-NMR (400 MHz, DMSO-d₆): δ 10.35, 8.73, 8.45, 7.70-7.80, 7.46, 7.30-7.40, 7.22, 7.16, 5.36, 5.21, 4.82, 4.55, 3.71, 3.33, 2.75, 2.66, 2.26; HRMS (FAB) found 530.2184 for C₂₉H₂₈FN₅O₄+H₁; Specific Rotation [α]²⁵_{D}=27 (c 0.60, DMSO).

### Example 25

### Preparation of N-(4-fluorobenzyl)-9-{cube root}[[(2S)-2-hydroxy-2-pyridin-3-ylethyl](methyl)amino]methyl}-1 methyl-2,7-dioxo-2,3-dihydro-1H,7H-pyrido[1,2,3-de]quinoxaline-6-carboxamide.

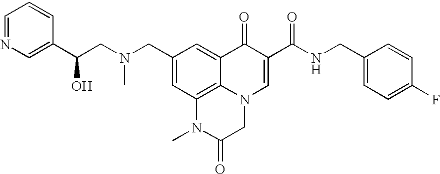

As described in the general procedure outlined in Example 9, 0.25 g of N-(4-fluorobenzyl)-9-(chloromethyl)-1-methyl-2,7-dioxo-2,3-dihydro-1H,7H-pyrido[1,2,3-de]quinoxaline-6-carboxamide, 0.18 g of S-3-(1-hydroxy-2-N-methylamino-ethyl)-pyridine, 0.21 mL of i-Pr₂NEt, and 0.30 g of 4 Å sieves in DMF (5 mL) affords 0.23 g of N-(4-fluorobenzyl)-9-{[[(2S)-2-hydroxy-2-pyridin-3-ylethyl](methyl) amino]methyl}-1-methyl-2,7-dioxo-2,3-dihydro-1H,7H-pyrido[1,2,3-de]quinoxaline-6-carboxamide as a pale yellow solid. Physical characteristics: mp 108-110° C.; 1H-NMR (400 MHz, DMSO-d₆): δ 10.21, 8.75, 8.51, 8.41, 7.75, 7.68, 7.32, 7.31, 7.22, 7.16, 5.37, 5.21, 4.81, 4.55, 3.70, 3.62, 3.34, 2.66, 2.57, 2.24; HRMS (FAB) found 530.2196 for C₂₉H₂₈FN₅O₄+H₁; Specific Rotation [α]25D=2 (c 0.90, DMSO).

### Example 26

### Preparation of rac 9-{([2-(1-benzofuran-2-yl)-2-hydroxyethyl](methyl)amino)methyl}-N-(4-chlorobenzyl)-1-(2-hydroxyethyl)-2,7-dioxo-2,3-dihydro-1H,7H-pyrido[1,2,3-de]quinoxaline-6-carboxamide.

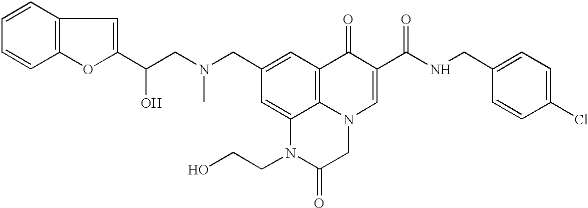

To a flask containing N-(4-chlorobenzyl)-9-(chloromethyl)-1-(2-hydroxyethyl)-2,7-dioxo-2,3-dihydro-1H,7H-pyrido[1,2,3-de]quinoxaline-6-carboxamide (0.20 g) and 1-(1-benzofuran-2-yl)-2-(methylamino)ethanol (0.13 g) is added DMF (3.5 mL) and diisopropylethylamine (0.7 mL). After stirring at room temperature overnight, the reaction mixture is diluted with ethyl acetate (75 mL), washed with dilute pH 4 phosphate buffer (25 mL), dilute pH 7 phosphate buffer (25 mL), brine, dried over sodium sulfate, filtered, and finally concentrated under reduced pressure. The residue is adsorbed onto silica and flash column chromatographed on silica eluting with 3% to 8% methanol in dichloromethane. The product containing fractions are combined and concentrated under reduced pressure. The resulting residue is crystallized from methanol-acetonitrile to yield 0.15 g of the title compound as a white solid. Physical characteristics: Mp 195-197° C.; ¹H NMR (d₆-DMSO) δ 10.4, 8.73, 7.79, 7.55, 7.4, 7.2, 6.73, 5.53, 5.18, 4.85, 4.57, 3.93, 3.8-3.6, 3.48, 2.9, 2.7, 2.29; HRMS (ESI+) m/z 615.2008 (M+H)⁺.

### Example 27

### Preparation of N-(4-fluorobenzyl)-9-{[[(2R)-2-hydroxy-2-(1,3-thiazol-2-yl)ethyl](methyl)amino]methyl}-1-methyl-2,7-dioxo-2,3-dihydro-1H,7H-pyrido[1,2,3-de]quinoxaline-6-carboxamide.

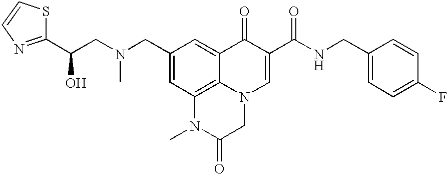

As described in the general procedure outlined in Example 9, 0.25 g of N-(4-fluorobenzyl)-9-(chloromethyl)-1-methyl-2,7-dioxo-2,3-dihydro-1H,7H-pyrido[1,2,3-de]quinoxaline-6-carboxamide, 0.19 g of R-2-(1-hydroxy-2-N-methylamino-ethyl)-thiazole, 0.21 mL of i-Pr2NEt, and 0.30 g of 4 Å sieves in DMF (5 mL) affords 0.21 g of N-(4-fluorobenzyl)-9-{[[(2R)-2-hydroxy-2-(1,3-thiazol-2-yl)ethyl](methyl)amino]methyl}-1-methyl-2,7-dioxo-2,3-dihydro-1H,7H-pyrido[1,2,3-de]quinoxaline-6-carboxamide as a pale yellow solid. Physical characteristics: mp 156-158° C.; ¹H-NMR (400 MHz, DMSO-d₆): δ 10.35, 8.73, 7.79, 7.22, 7.62, 7.35-7.45, 7.16, 6.18, 5.21, 5.06, 4.55, 3.61, 3.36, 2.85, 2.74, 2.26; HRMS (FAB) found 536.1750 for C₂₇H₂₆FN₅O₄S+Hl; Specific Rotation [α]²⁵_{D}=13 (c 0.99, DMSO).

### Example 28

### Preparation of N-(4-fluorobenzyl)-9-{[[(2S)-2-hydroxy-2-thien-3-ylethyl](methyl)amino]methyl}-1-methyl-2,7-dioxo-2,3-dihydro-1H,7H-pyrido[1,2,3-de]quinoxaline-6-carboxamide.

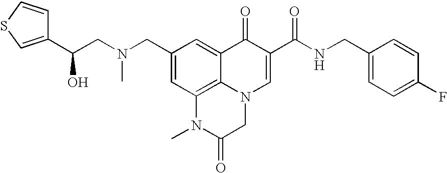

As described in the general procedure outlined in Example 9, 0.25 g of N-(4-fluorobenzyl)-9-(chloromethyl)-1-methyl-2,7-dioxo-2,3-dihydro-1H,7H-pyrido[1,2,3-de]quinoxaline-6-carboxamide, 0.19 g of S-3-(1-hydroxy-2-N-methylamino-ethyl)-thiophene, 0.21 mL of i-Pr₂NEt, and 0.30 g of 4 Å sieves in DMF (5 mL) affords 0.20 g of N-(4-fluorobenzyl)-9-{[[(2S)-2-hydroxy-2-thien-3-ylethyl](methyl)amino]methyl}-1-methyl-2,7-dioxo-2,3-dihydro-1H,7H-pyrido [1,2,3-de]quinoxaline-6-carboxamide as a pale yellow solid. Physical characteristics: mp 150-152° C.; ¹H-NMR (400 MHz, DMSO-d₆): δ 10.33, 8.73, 7.95, 7.80, 7.43, 7.35-7.42, 7.31, 7.17, 7.05, 5.21, 5.14, 4.85, 4.55, 3.69, 3.35, 2.63, 2.24; HRMS (FAB) found 535.1799 for C₂₈H₂₇FN₄O₄S+H₁; Specific Rotation [α]²⁵_{D}=6 (c 1.00, DMSO).

### Example 29

### Preparation of N-(4-fluorobenzyl)-9-{[[(2S)-2-hydroxy-2-phenylethyl](methyl)amino]methyl}-1-methyl-2,7-dioxo-2,3-dihydro-1H,7H-pyrido[1,2,3-de]quinoxaline-6-carboxamide.

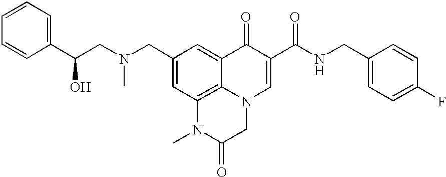

As described in the general procedure outlined in Example 9, 0.25 g of N-(4-fluorobenzyl)-9-(chloromethyl)-1-methyl-2,7-dioxo-2,3-dihydro-1H,7H-pyrido[1,2,3-de]quinoxaline-6-carboxamide, 0.18 g of S-α-[(methylamino)methyl]-benzenemethanol, 0.21 mL of i-Pr₂NEt, and 0.30 g of 4 Å sieves in DMF (5 mL) affords 0.20 g of N-(4-fluorobenzyl)-9-{[[(2S)-2-hydroxy-2-phenylethyl](methyl)amino]methyl}-1-methyl-2,7-dioxo-2,3-dihydro-1H,7H-pyrido[1,2,3-de]quinoxaline-6-carboxamide as a pale yellow solid. Physical characteristics: mp 150-152°; ¹H-NMR (400 MHz, DMSO-d₆): δ 10.32, 8.73, 7.95, 7.86, 7.12-7.42, 5.21, 5.15, 4.76, 4.55, 3.69, 3.38, 3.31, 2.60, 2.53, 2.25; HRMS (FAB) found 529.2239 for C₃₀H₂₉FN₄O₄+H₁; Specific Rotation [α]²⁵_{D}=10 (c 0.94, DMSO).

### Example 30

### Preparation of N-(4-fluorobenzyl)-9-{[[(2R)-2-hydroxy-2-pyrazin-2-ylethyl](methyl)amino]methyl}-1-methyl-2,7-dioxo-2,3-dihydro-1H,7H-pyrido[1,2,3-de]quinoxaline-6-carboxamide.

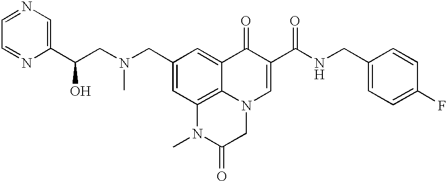

As described in the general procedure outlined in Example 9, 0.25 g of N-(4-fluorobenzyl)-9-(chloromethyl)-1-methyl-2,7-dioxo-2,3-dihydro-1H,7H-pyrido[1,2,3-de]quinoxaline-6-carboxamide, 0.18 g of R-2-(1-hydroxy-2-N-methylamino-ethyl)-pyrazine, 0.21 mL of i-Pr₂NEt, and 0.30 g of 4 Å sieves in DMF (5 mL) affords 0.28 g of N-(4-fluorobenzyl)-9-{[[(2R)-2-hydroxy-2-pyrazin-2-ylethyl](methyl)amino]methyl}-1-methyl-2,7-dioxo-2,3-dihydro-1H,7H-pyrido[1,2,3-de]quinoxaline-6-carboxamide as an ivory solid. Physical characteristics: mp 108-110° C.; ¹H-NMR (400 MHz, DMSO-d₆): δ 10.35, 8.72, 8.70, 8.50, 7.25, 7.38, 7.21, 7.16, 5.62, 5.21, 4.87, 4.55, 3.67, 3.34, 3.31, 2.82, 2.73, 2.24; HRMS (FAB) found 531.2152 for C₂₈H₂₇FN₆O₄+H₁; Specific Rotation [α]²⁵_{D}=25 (c 0.94, DMSO).

### Example 31

### Preparation of N-(4-chlorobenzyl)-9-{([(2R)-2-(2-furyl)-2-hydroxyethyl](methyl)amino)methyl}-1-(2-hydroxyethyl)-2,7-dioxo-2,3-dihydro-1H,7H-pyrido[1,2,3-de]quinoxaline-6-carboxamide.

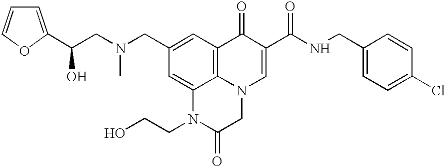

To a flask containing N-(4-chlorobenzyl)-9-(chloromethyl)-1-(2-hydroxyethyl)-2,7-dioxo-2,3-dihydro-1H,7H-pyrido[1,2,3-de]quinoxaline-6-carboxamide (0.16 g) and (1R)-1-(2-furyl)-2-(methylamino)ethanol (0.07 g) is added DMF (3 mL) and diisopropylethylamine (0.6 mL). After stirring at room temperature for 12 days, the reaction mixture is diluted with ethyl acetate (60 mL), washed with dilute pH 4 phosphate buffer (2×25 mL), brine, dried over sodium sulfate, filtered, and finally concentrated under reduced pressure. The residue is adsorbed onto silica and flash column chromatographed on silica eluting with 3% to 9% methanol in dichloromethane. The product containing fractions are combined and concentrated under reduced pressure. The resulting residue is crystallized from acetonitrile to yield 0.13 g of the title compound as a tan solid. Physical characteristics: Mp 166-167° C.; ¹H NMR (d₆-DMSO) δ 10.4, 8.74, 7.80, 7.55, 7.48, 6.38, 6.27, 5.24, 5.21, 4.88, 4.75, 4.56, 4.1, 3.7-3.6, 2.7, 2.21; HRMS (ESI+) m/z 565.1866 (M+H)⁺; Anal. Found: C, 61.46; H, 5.24; N, 9.99.

### Example 32

### Preparation of N-(4-fluorobenzyl)-9-{[[(2R)-2-hydroxy-2-thien-2-ylethyl](methyl)amino]methyl}-1-methyl-2,7-dioxo-2,3-dihydro-1H,7H-pyrido[1,2,3-de]quinoxaline-6-carboxamide.

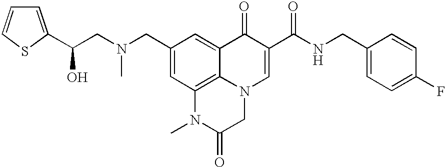

As described in the general procedure outlined in Example 9, 0.25 g of N-(4-fluorobenzyl)-9-(chloromethyl)-1-methyl-2,7-dioxo-2,3-dihydro-1H,7H-pyrido[1,2,3-de]quinoxaline-6-carboxamide, 0.19 g of R-2-(1-hydroxy-2-N-methylamino-ethyl)-thiophene, 0.21 mL of i-Pr₂NEt, and 0.30 g of 4 Å sieves in DMF (5 mL) affords 0.19 g of N-(4-fluorobenzyl)-9-{[[(2R)-2-hydroxy-2-thien-2-ylethyl](methyl) amino]methyl}-1-methyl-2,7-dioxo-2,3-dihydro-1H,7H-pyrido[1,2,3-de]quinoxaline-6-carboxamide as an ivory solid. Physical characteristics: mp 113-115° C.; ¹H-NMR (400 MHz, DMSO-d₆): δ 10.32, 8.73, 7.95, 7.80, 7.30-7.40, 7.16, 6.99, 6.95, 5.56, 5.21, 4.96, 4.55, 3.75, 3.68, 3.34, 2.66, 2.60, 2.24; HRMS (FAB) found 535.1826 for C₂₈H₂₇FN₄O₄S+H₁.

### Example 33

### Preparation of rac 9-{[[2-(1-benzofuran-2-yl)-2-hydroxyethyl](methyl)amino]methyl}-N-(4-fluorobenzyl)-1-methyl-2,7-dioxo-2,3-dihydro-1H,7H-pyrido[1,2,3-de]quinoxaline-6-carboxamide.

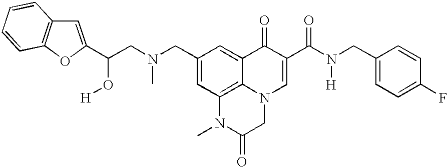

As described in the general procedure outlined in Example 9, 0.25 g of N-(4-fluorobenzyl)-9-(chloromethyl)-1-methyl-2,7-dioxo-2,3-dihydro-1H,7H-pyrido[1,2,3-de]quinoxaline-6-carboxamide, 0.23 g of (±)-1-(1-benzofuran-2-yl)-2-(methylamino)ethanol, 0.21 mL of i-Pr₂NEt, and 0.30 g of 4 Å sieves in DMF (5 mL) affords 0.19 g of rac 9-{[[2-(1-benzofuran-2-yl)-2-hydroxyethyl](methyl)amino]methyl}-N-(4-fluorobenzyl)-1-methyl-2,7-dioxo-2,3-dihydro-1H,7H-pyrido[1,2,3-de]quinoxaline-6-carboxamide as a white solid. Physical characteristics: mp161-163° C.; ¹H-NMR (400 MHz, DMSO-d₆): δ 10.35, 8.72, 7.76, 7.53, 7.30-7.42, 7.10-7.22, 6.73, 5.55, 5.17, 4.87, 4.55, 3.68, 3.60, 3.35, 2.88, 2.69, 2.29; HRMS (FAB) found 569.2214 for C₃₂H₂₉FN₄O₅+H₁.

### Example 34

### Preparation of 9-{[[(2R)-2-(1-benzofuran-2-yl)-2-hydroxyethyl](methyl)amino]methyl)-N-(4-chlorobenzyl)-1-methyl-2,7-dioxo-2,3-dihydro-1H,7H-pyrido[1,2,3-de]quinoxaline-6-carboxamide.

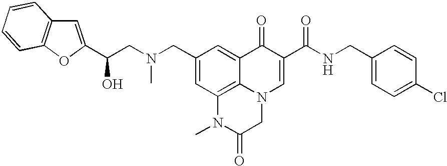

As described in the general procedure outlined in Example 9, 0.56 g of N-(4-chlorobenzyl)-9-(chloromethyl)-1-methyl-2,7-dioxo-2,3-dihydro-1H,7H-pyrido[1,2,3-de]quinoxaline-6-carboxamide, 0.50 g of (R)-1-(1-benzofuran-2-yl)-2-(methylamino)ethanol, 0.46 mL of i-Pr₂NEt, and 0.65 g of 4 Å sieves in DMF (35 mL) affords 0.69 g of 9-{[[(2R)-2-(1-benzofuran-2-yl)-2-hydroxyethyl](methyl)amino]methyl}-N-(4-chlorobenzyl)-1-methyl-2,7-dioxo-2,3-dihydro-1H,7H-pyrido[1,2,3-de]quinoxaline-6-carboxamide as a pale yellow solid. Physical characteristics: mp 86-87° C.; ¹H-NMR (400 MHz, DMSO-d₆): δ 10.35, 8.71, 7.76, 7.54, 7.38, 7.20, 7.11, 6.74, 5.56, 5.17, 4.87, 5.56, 3.68, 3.59, 2.99, 2.90, 2.71, 2.29; HRMS (FAB) found 585.1890 for C₃₂H₂₉CLN₄O₅+H₁; Specific Rotation [α]²⁵_{D}=−7 (c 0.97, DMSO).

### Example 35

### Preparation of 9-{[[(2S)-2-(1-benzofuran-2-yl)-2-hydroxyethyl](methyl)amino]methyl}-N-(4-chlorobenzyl)-1-methyl-2,7-dioxo-2,3-dihydro-1H,7H-pyrido[1,2,3-de]quinoxaline-6-carboxamide.

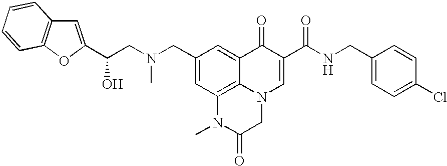

As described in the general procedure outlined in Example 9, 0.56 g of N-(4-chlorobenzyl)-9-(chloromethyl)-1-methyl-2,7-dioxo-2,3-dihydro-1H,7H-pyrido[1,2,3-de]quinoxaline-6-carboxamide, 0.50 g of (S)-1-(1-benzofuran-2-yl)-2-(methylamino)ethanol, 0.46 mL of i-Pr₂NEt, and 0.65 g of 4 Å sieves in DMF (35 mL) affords 0.70 g of 9-f{[[(2S)-2-(1-benzofuran-2-yl)-2-hydroxyethyl](methyl)amino]methyl}-N-(4-chlorobenzyl)-1-methyl-2,7-dioxo-2,3-dihydro-1H,7H-pyrido[1,2,3-de]quinoxaline-6-carboxamide as a pale yellow solid. Physical characteristics: mp 190-191° C.; ¹H-NMR (400 MHz, DMSO-d₆): δ 10.35, 8.71, 7.76, 7.54, 7.38, 7.20, 7.11, 6.74, 5.56, 5.17, 4.87, 5.56, 3.68, 3.59, 2.99, 2.90, 2.71, 2.29; HRMS (FAB) found 585.1912 for C₃₂H₂₉CLN₄O₅+H₁; Specific Rotation [α]²⁵_{D}=7 (c 0.99, DMSO).

### Example 36

### Preparation of 9-{[[(2R)-2-(1-benzofuran-2-yl)-2-hydroxyethyl](methyl)amino]methyl}-N-(4-fluorobenzyl)-1-methyl-2,7-dioxo-2,3-dihydro-1H,7H-pyrido[1,2,3-de]quinoxaline-6-carboxamide.

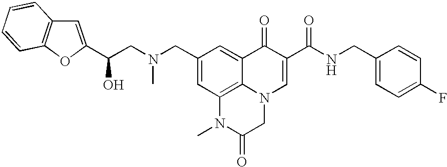

As described in the general procedure outlined in Example 9, 0.28 g of N-(4-fluorobenzyl)-9-(chloromethyl)-1-methyl-2,7-dioxo-2,3-dihydro-1H,7H-pyrido[1,2,3-de]quinoxaline-6-carboxamide, 0.26 g of (R)-1-(1-benzofuran-2-yl)-2-(methylamino)ethanol, 0.24 mL of i-Pr₂NEt, and 0.34 g of 4 Å sieves in DMF (30 mL) affords 0.26 g of 9-{[[(2R)-2-(1-benzofuran-2-yl)-2-hydroxyethyl](methyl)amino]methyl}-N-(4-fluorobenzyl)-1-methyl-2,7-dioxo-2,3-dihydro-1H,7H-pyrido [1,2,3-de]quinoxaline-6-carboxamide as a white solid. Physical characteristics: mp 94-96° C.; ¹H-NMR (400 MHz, CDCl₃): δ 10.25, 8.63, 7.98, 7.50, 7.15-7.30, 7.01, 6.65, 4.97, 4.95, 4.64, 3.82, 3.70, 3.39, 3.02, 2.87, 2.41; HRMS (ESI) found 569.2190 for C₃₂H₂₉N₄O₅F+H₁; Specific Rotation [α]²⁵_{D}=−7 (c 0.68, DMSO).

### Example 37

### Preparation of 9-{[[(2S)-2-(1-benzofuran-2-yl)-2-hydroxyethyl](methyl)amino]methyl}-N-(4-fluorobenzyl)-1-methyl-2,7-dioxo-2,3-dihydro-1H,7H-pyrido[1,2,3-de]quinoxaline-6-carboxamide.

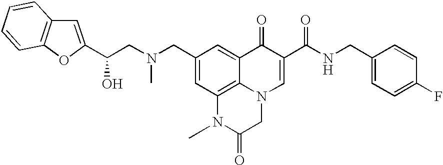

As described in the general procedure outlined in Example 9, 0.25 g of N-(4-fluorobenzyl)-9-(chloromethyl)-1-methyl-2,7-dioxo-2,3-dihydro-1H,7H-pyrido [1,2,3-de]quinoxaline-6-carboxamide, 0.23 g of (S)-1-(1-benzofuran-2-yl)-2-(methylamino)ethanol, 0.24 mL of i-Pr₂NEt, and 0.30 g of 4 Å sieves in DMF (30 mL) affords 0.22 g of 9-{[[(2S)-2-(1-benzofuran-2-yl)-2-hydroxyethyl](methyl)amino]methyl}-N-(4-fluorobenzyl)-1-methyl-2,7-dioxo-2,3-dihydro-1H,7H-pyrido[1,2,3-de]quinoxaline-6-carboxamide as a pale yellow solid. Physical characteristics: mp 195-197° C.; ¹H-NMR (400 MHz, CDCl₃): δ 10.25, 8.67, 8.01, 7.52, 7.20-7.40, 7.03, 6.68, 4.90-5.10, 4.65, 3.86, 3.75, 3.45, 3.04, 2.90, 2.45; HRMS (ESI) found 569.2217 for C₃₂H₂₉N₄O₅F+H₁; Specific Rotation [α]²⁵_{D}=7 (c 0.93, DMSO).

Preparations of Intermediates:

(1) Preparation of 1-(1-benzofuran-2-yl)-2-(methylamino)ethanol.

KOH (9.2 g), and water (3.7 mL) are added to acetonitrile (125 mL). Benzofuran-2-carbaldehyde (12.0 g) is dissolved into the acetonitrile mixture. Trimethylsulfonium iodide (16.7 g) is added and the mixture is stirred at 60° C. for 3 h. The reaction mixture is cooled to room temperature and filtered. The filtrant is washed with diethyl ether and filtered. This process is repeated until no more solid precipitates. The solution is concentrated in vacuo and the resulting residue is dissolved in a solution of methylamine in methanol (2.0 M, 410 mL). The mixture is stirred at room temperature for 18 hours, then concentrated in vacuo to a brown oil. The oil is purified by column chromatography (MeOH/CHCl₃, 5%, 10%; NH-₄OH/MeOH/CHCl₃, 1:10:89, 1:20:79) to afford 3.0 g of the title compound as an off-white solid. Physical characteristics: ¹H NMR (300 MHz, DMSO-d₆) δ 7.59, 7.53, 7.24, 6.47, 5.62, 4.77, 2.83, 2.32.

(2) Preparation of 1-(3a,7a-dihydro-1-benzothien-3-yl)-2-(methylamino)ethanol.

Potassium hydroxide (3.11 g) and H₂O (0.12 mL) are added to acetonitrile (50 mL). Trimethylsulfonium iodide (5.65 g) and thianaphthene-3-carboxaldehyde (4.50 g) are then added. The reaction mixture is heated to 60° C. for 4 h. The reaction mixture is allowed to cool to room temperature and is then diluted with Et₂O (25 mL). The precipitate is filtered off, and the filtrate is concentrated in vacuo. The resulting crude material is dissolved in methanol (40 mL) and added to a solution of methylamine in methanol (2M, 100 mL). The reaction mixture is stirred at room temperature for 3 d. The reaction mixture is concentrated in vacuo. The resulting brown oil is purified via column chromatography (CHCl₃/methanol, 95/5, 90/10; CHCl₃/methanol/NH₄OH, 90/10/1) to yield 1.75 g of the title compound as a yellow solid. Physical characteristics: Mp 98-102° C.; ¹H NMR (400 MHz, DMSO-d₆) δ 7.98-7.90, 7.51, 7.60, 7.40-7.33, 5.43, 5.04, 2.80, 2.34; MS (ESI+) m/z 208 (M+H)⁺.

(3) Preparation of 1-(2,5-dimethyl-3-furyl)-2-(methylamino)ethanol.

3-acetyl-2,5-dimethylfuran (13.3 mL) is dissolved in ½ dioxane/Et₂O (600 mL) and cooled to 0° C. Bromine (16.0 g) is added dropwise over 1 h. The reaction mixture is stirred at 0° C. for 1 h and then allowed to warm to room temperature. The reaction mixture is stirred at room temperature for 18 h. The reaction mixture is cooled to 0° C. and an additional 1.0 mL of bromine is added. The reaction mixture is allowed to warm to room temperature and is stirred for 2 h. The reaction is quenced with a saturated ammonium chloride solution (100 mL). The organic layer is removed and the aqueous layer extracted with Et₂O (2×100 mL). The combined organic layers are dried (MgSO₄), filtered, and concentrated in vacuo. The resulting brown, oily solid is purified via column chromatography (hexanes/CH₂Cl₂; 70/30) to yield a yellow oily solid which is recrystallized from hexanes to yield 7.52 g of 2-bromo-1-(2,5-dimethyl-3-furyl)ethanone as a white solid. Physical characteristics: Mp 56-58° C.; ¹H NMR (400 MHz, DMSO-d₆) δ 6.49, 4.54, 2.50, 2.08; ¹³C NMR (100 MHz, CDCl₃) δ 187.2, 159.3, 150.5, 118.9, 105.6, 33.2, 14.4, 13.2.

2-bromo-1-(2,5-dimethyl-3-furyl)ethanone (7.30 g) is dissolved in methanol (80 mL) and added dropwise to a solution of methylamine in methanol (2M, 168 mL) at 0° C. The reaction mixture is stirred at 0° C. for 30 min and then sodium borohydride (1.91 g) in H₂O (40 mL) is added dropwise. The reaction mixture is stirred at 0° C. for 1.5 h and then allowed to warm to room temperature. The reaction mixture is stirred at room temperature for 18 h. An additional 0.636 g of sodium borohydride is added and stirring is continued for 3 h. The reaction is quenched with a 1N HCl solution and concentrated in vacuo to remove methanol. The residue is poured into cold 2 N NaOH (100 mL)/ethyl acetate (200 mL). The organic layer is removed and the aqueous layer extracted with ethyl acetate (3×200 mL). The combined organic layers are dried (MgSO₄), filtered, and concentrated in vacuo. The resulting yellow oil is purified via column chromatography (CHCl₃/methanol, 95/5, 90/10; CHCl₃/methanol/NH₄OH, 90/10/1). The resulting pale yellow solid is recrystallized from ethyl acetate to yield 2.406 g of the title compound as a white solid. Physical characteristics: Mp 76-77° C.; ¹H NMR (400 MHz, DMSO-d₆) δ 5.93, 4.82, 4.47-4.43, 2.64-2.54, 2.46-2.42, 2.32, 2.16; MS (ESI+) m/z 170 (M+H)⁺.

(4) Preparation of 1-(methylamino)-3-phenylpropan-2-ol.

(2,3-epoxypropyl)benzene (5.00 g) is added to a solution of methylamine in methanol (2.0 M, 187 mL). The reaction mixture is stirred at room temperature for 18 h and then concentrated in vacuo. The resulting yellow oil is purified via column chromatography (CH₂Cl₂/methanol, 95/5, 50/50) to yield 3.89 g of the title compound as a yellow oil. Physical characteristics: ¹H NMR (400 MHz, DMSO-d₆)δ) 7.28-7.15, 4.57, 3.74-3.68, 2.72-2.58, 2.43-2.36, 2.26; MS (ESI+) m/z 166 (M+H)⁺.

(5) Preparation of 2-(methylamino)-1-quinolin-2-ylethanol.

Potassium hydroxide (3.21 g) and H₂O (0.13 mL) are added to acetonitrile (50 mL). Trimethylsulfonium iodide (5.84 g) and 2-quinoline carboxaldehyde (4.50 g) are then added. The reaction mixture is heated to 60° C. for 4 h. The reaction mixture is allowed to cool to room temperature and is diluted with Et₂O (25 mL). The precipitate is filtered off. The filtrate is concentrated in vacuo and the residue is re-subjected to the reaction conditions above and heated to 60° C. for 1 h. The reaction mixture is allowed to cool to room temperature and is diluted with Et2O (25 mL). The precipitate is filtered off and the filtrate is concentrated in vacuo. The resulting crude material is dissolved in methanol (20 mL) and added to a 2.0 M solution of methylamine in methanol (100 mL). The reaction mixture is heated to reflux for 1 h. The reaction mixture is allowed to cool to room temperature and concentrated in vacuo. The resulting brown oil is purified via column chromatography (CHCl₃/methanol, 95/5, 90/10; CHCl₃/methanol/NH₄OH, 90/10/1) to yield 1.191 g of the title compound as a yellow-green oil. Physical characteristics: ¹H NMR (400 MHz, DMSO-d₆) δ 8.36-8.33, 7.98-7.94, 7.76-7.67, 7.59-7.54, 5.63, 4.88-4.84, 2.89-2.72, 2.32; MS (ESI+) m/z 203 (M+H)⁺.

(6) Preparation of 2-(methylamino)-1-(5-methyl-2-furyl)ethanol.

Bromine (5.1 mL) is added dropwise over 1 h to a solution of 2-acetyl-5-methylfuran (11.0 g) in dioxane/Et₂O (½, 60 mL) at 0° C. (internal). The reaction mixture is stirred at 0° C. for 30 min and then allowed to warm to room temperature and is stirred for 18 h. The reaction mixture is cooled to 0° C. (internal), and additional bromine (1.53 mL) is added dropwise. The reaction mixture is allowed to warm to room temperature and is stirred for 1 h. A saturated ammonium chloride solution (100 mL) is added. The organic layer is removed and the aqueous layer is extracted with Et₂O (2×100 mL). The combined organic layers are dried (MgSO₄), filtered, and concentrated in vacuo. The resulting brown solid is purified via column chromatography (hexanes/CH₂Cl₂, 70/30) to yield a yellow solid which is recrystallized from EtOAc/hexanes to yield 8.571 g of 2-bromo-1-(5-methyl-2-furyl)ethanone as a pale yellow solid. Physical characteristics: Mp 60-63° C.; ¹H NMR (400 MHz, DMSO-d₆) δ 7.60, 6.44, 4.58, 2.41.

A solution of 2-bromo-1-(5-methyl-2-furyl)ethanone (8.00 g) in methanol (100 mL) is added dropwise to a 2.0 M solution of methylamine in methanol (197 mL) at 0° C. (internal). The reaction mixture is stirred at 0° C. for 30 min. A solution of sodium borohydride (2.23 g) in H₂O (40 mL) is then added dropwise. The reaction mixture is stirred at 0° C. for 1.5 h and then quenched with a 2 N HCl solution (to pH 3-4). The reaction mixture is concentrated in vacuo to remove methanol and then poured into cold EtOAc (200 mL)/2 N NaOH (100 mL). The organic layer is removed. The aqueous layer is adjusted to pH 12 with a 2 N NaOH solution and extracted with EtOAc (3×200 mL). The combined organic layers are dried (MgSO₄), filtered, and concentrated in vacuo. The resulting yellow oil is purified via column chromatography (CHCl₃/methanol, 95/5, 90/10; CHCl₃/methanol/NH₄OH, 90/10/1). The resulting yellow oil is crystallized from diethyl ether to yield 1.88 g of the title compound as a yellow solid. Physical characteristics: Mp 40-45° C.; ¹H NMR (400 MHz, DMSO-d₆) δ 6.11, 5.97-5.96, 5.05, 4.54-4.51, 2.72-2.65, 2.29, 2.22; MS (ESI+) m/z 156 (M+H)⁺.

(7) Preparation of 1-(2-Furyl)-2-(methylamino)ethanol.

Bromine (6.5 mL) is added dropwise over 1 h to a solution of 2-acetylfuran (11.0 g) in dioxane/Et₂O (½, 60 mL) at 0° C. (internal). The reaction mixture is then allowed to warm to room temperature and is stirred for 2 h. A saturated ammonium chloride solution (70 mL) is added. The organic layer is removed and the aqueous layer is extracted with diethyl ether (2×50 mL). The combined organic layers are dried (MgSO₄), filtered, and concentrated in vacuo. The resulting brown solid is purified by column chromatography (hexanes/CH₂Cl₂, 70/30) to yield 7.996 g of 2-furoyl bromide as a yellow oil. Physical characteristics: ¹H NMR (400 MHz, DMSO-d₆) δ 8.09, 7.66-7.64, 6.79-6.77, 4.65.

A solution of 2-furoyl bromide (7.50 g) in methanol (40 mL) is added dropwise to a 2.0 M solution of methylamine in methanol (198 mL) at 0° C. (internal). The reaction mixture is stirred at 0° C. for 30 min. A solution of sodium borohydride (2.25 g) in water (40 mL) is then added dropwise. The reaction mixture is stirred at 0° C. for 30 min and then quenched with a 2 N HCl solution (to pH 3-4). The reaction mixture is concentrated in vacuo to remove methanol and is then poured into cold EtOAc (200 mL)/2 N NaOH (100 mL). The organic layer is removed. The aqueous layer is adjusted to pH 12 with a 2 N NaOH solution and extracted with EtOAc (3×200 mL). The combined organic layers are dried (MgSO₄), filtered, and concentrated in vacuo. The resulting brown oil is purified by column chromatography (CHCl₃/methanol, 95/5; CHCl₃/methanol/NH₄OH, 90/10/1) to yield 2.06 g of the title compound as a brown oil. Physical characteristics: ¹H NMR (400 MHz, DMSO-d₆) δ 7.56, 6.39-6.37, 6.26-6.25, 5.15, 4.62-4.58, 2.77-2.66, 2.33; MS (ESI+) m/z 142 (M+H)⁺.

(8) Preparation of 1-(3-Furyl)-2-(methylamino)ethanol.

Trimethylsulfonium iodide (20.4 g) and 3-furaldehyde (8.65 mL) are added to potassium hydroxide (11.2 g) and H₂O (0.45 mL) in acetonitrile (150 mL). The reaction mixture is heated to 60° C. for 2.5 h. The reaction mixture is allowed to cool to room temperature. The precipitate is filtered off, and the filtrate is concentrated in vacuo. The resulting crude material (10.747 g) is dissolved in methanol (50 mL) and added to a 2.0 M solution of methylamine in methanol (100 mL). The reaction mixture is stirred at room temperature for 3 d and then heated to reflux for 30 min. The reaction mixture is allowed to cool to room temperature and is concentrated in vacuo. The resulting brown oil is purified via column chromatography (CHCl₃/methanol, 95/5, 90/10; CHCl₃/methanol/NH₄OH, 90/10/1) to yield 2.703 g of the title compound as a yellow oil. Physical characteristics: ¹H NMR (400 MHz, DMSO-d₆) δ 7.56-7.55, 7.51, 6.44, 5.07, 4.58-4.55, 2.62-2.56, 2.30; MS (ESI+) m/z 142 (M+H)⁺.

(9) Preparation of 2-(methylamino)-1-(6-methylpyridin-2-yl)ethanol.

Potassium hydroxide (11.2 g) and H₂O (0.45 mL) are added to acetonitrile (150 mL). Trimethylsulfonium iodide (20.4 g) and 6-methyl-2-pyridine carboxaldehyde (12.1 g) are then added. The reaction mixture is heated to 60° C. for 3 h. The reaction mixture is allowed to cool to room temperature. The precipitate is filtered and the filtrate is concentrated in vacuo. The resulting crude material (13.5 g) is dissolved in methanol (50 mL) and added to a 2.0 M solution of methylamine in methanol (250 mL). The reaction mixture is heated to reflux for 30 min. The reaction mixture is concentrated in vacuo. The resulting brown oil is purified via column chromatography (CHCl₃/methanol, 95/5, 90/10; CHCl₃/methanol/NH₄OH, 90/10/1). The resulting brown oil is suspended in hot methanol and the insoluble material filtered off. The filtrate is concentrated in vacuo to yield 3.657 g of the title compound as a yellow solid. Physical characteristics: Mp 33-38° C.; ¹H NMR (400 MHz, DMSO-d₆) δ 7.64, 7.29, 7.10, 5.40, 4.63-4.60, 2.79-2.75, 2.61-2.54, 2.43, 2.30; MS (ESI+) m/z 167 (M+H)⁺.

(10) Preparation of 1-(3-methoxyphenyl)-2-(methylamino)ethanol.

A sealed tube containing 2-(3-methoxyphenyl)oxirane (J. Med. Chem. 1992, 35, 3045) (4.0 g) methylamine (27 mL, 2M solution in methanol) are heated for 4 hrs at 100° C. After cooling, the reaction is concentrated under reduced pressure and the residual oil is purified by silica gel chromatography (Biotage 40M, 6% methanol/dichloromethane) to yield the title compound (1.1 g) as an off-white solid. Physical characteristic: ¹H NMR (400 MHz, CDCl₃) δ 7.28, 6.97, 6.81, 4.77, 3.83, 2.99, 2.80, 2.49.

(11) Preparation of 5-[1-Hydroxy-2-(methylamino)ethyl]thiophene-2-carbonitrile.

To a solution of 2-acetyl-5-cyanothiophene (1.5 g) in 20 mL of p-dioxane/ethyl ether (1:2, v/v) is added 0.50 mL of bromine. The reaction is stirred at room temperature for 2 hours. Ice water (30 mL) is added. The resulting solid is collected by filtration and is washed with water to yield 1.4 g of 5-(bromoacetyl)thiophene-2-carbonitrile as a white solid. The filtrate is allowed to stand overnight to yield 0.86 g of 5-(bromoacetyl)thiophene-2-carbonitrile as a white solid. Physical characteristics: ¹H NMR (DMSO-d₆) δ 8.16, 8.11, 4.94; MS (ESI−) m/z 230 (M−H)⁺.

To a mixture of 5-(bromoacetyl)thiophene-2-carbonitrile (1.85 g) in 50 mL of methanol at −10° C. is added NaBH₄ (0.46 g) in 5 mL of water. After stirring for 10 minutes, HBr is added to adjust the pH to 3. The reaction mixture is concentrated to approximately 25 mL before water (30 mL) is added. The mixture is extracted with dichloromethane (3×40 mL). The organic phases are combined, washed with brine, and dried over MgSO₄ and concentrated to give 5-(2-bromo-1-hydroxyethyl)thiophene-2-carbonitrile (1.6 g) as an orange oil. Physical characteristics: ¹H NMR (DMSO-d₆) δ 7.86, 7.23, 6.67, 5.17, 3.81, 3.68; MS (ESI−) m/z 232 (M−H)⁺.

To a solution of 5-(2-bromo-1-hydroxyethyl)thiophene-2-carbonitrile (1.6 g) in 20 mL of methanol is added 80 mL of methylamine solution (2.0 M in methanol). The reaction mixture is stirred at room temperature overnight. The reaction is concentrated and the resulting residue is dissolved in 20 mL of methanol and treated with BioRad AG® 50 w-x2 resin (2 g, hydrogen form, strongly acidic cation) for 4 hours. The resin is collected by filtration and is washed with methanol. The resin-bound product is eluted off with 10% NH₄OH/MeOH (100 mL). The ammonium hydroxide filtrate is concentrated under reduced pressure. The residue is flash column chromatographed on silica gel eluting with 1% NH₄OH/10% MeOH/89% CH₂Cl₂ to yield 0.80 g of the title compound as a white solid. Physical characteristics: ¹H NMR (DMSO-d₆) δ 7.81, 7.13, 6.13, 4.93, 2.72, 2.33; MS (ESI+) m/z 183 (M+H)⁺; HRMS found 183.0600.

(12) Preparation of 2-(methylamino)-1-pyrimidin-2-ylethanol.

2-Acetylpyrimidine (Chimia 1996, 50, 538 and J. Het. Chem. 1994, 31, 1041) (7.37 g) and diisopropylethylamine (23.4 g) are dissolved in dry CH₂Cl₂ under N₂ then cooled in an ice bath. Triisopropylsilyl trifluoromethanesulfonate (17.9 mL) is added over 2-3 min and the reaction is stirred overnight. The solvent is removed under reduced pressure and the residue is treated with ether (200 mL), filtered and washed with saturated sodium bicarbonate solution (2×50 mL). Evaporation of the solvent gives 2-{1-[(triisopropylsilyl)oxy]vinyl}pyrimidine as a red oil. Physical characteristics: HRMS (FAB) found 279.1898 (M+H)⁺; ¹H NMR (300 MHz, CDCl₃) δ 1.15, 1.31, 4.90, 5.82, 7.16, 8.74.

N-chlorosuccinimide (9.97 g) is added to a solution of {1-[(triisopropylsilyl)oxy]vinyl}pyrimidine (17.3 g) in dry THF (120 mL) under N₂ then heated at 65° C. for 5 h. After cooling, ether (275 mL) is added and then washed with saturated sodium bicarbonate solution (2×100 mL). The organic layer is dried over sodium sulfate, filtered and concentrated under reduced pressure. The resulting oil is dissolved in hexanes (250 mL), treated with MgSO₄ and filtered. Concentration at reduced pressure affords 2-{2-chloro-1-[(triisopropylsilyl)oxy]ethenyl}pyrimidine as a yellow oil. Physical characteristics: HRMS (FAB) found 313.1509(M+H)⁺; ¹H NMR (300 MHz, CDCl₃) δ 1.13, 1.33, 6.97, 7.17, 8.68.

2-{2-Chloro-1-[(triisopropylsilyl)oxy]ethenyl}pyrimidine (19.4 g) is dissolved in acetonitrile (90 mL) and treated with 48% HF (10 mL) for 4 h. Saturated sodium bicarbonate solution (ca. 250 mL) is then added carefully to adjust the pH to ˜7 and the mixture is extracted with CH₂Cl₂ (3×200 mL). The combined organic layers are dried (Na₂SO₄), filtered and concentrated. The resulting biphasic mixture is decanted to remove the upper phase and the lower oil is chromatographed over silica gel eluting with 2.5% MeOH—CHCl₃ to give 6.5 g of 2-chloro-1-pyrimidin-2-ylethanone as a pale yellow solid. Physical characteristics: mp 73-80° C.; Anal. Found: C, 46.05; H, 3.09; N, 17.93.

2-Chloro-1-pyrimidin-2-ylethanone (6.15 g) is dissolved in ethanol (125 mL) and CeCl₃.7H₂O (14.64 g) is added. The mixture is stirred for 10 min, then sodium borohydride (1.49 g) is added over 2 min. After 1 hr, the solid is filtered and the filtrate evaporated. The resulting residue is treated with saturated ammonium chloride solution (25 mL), followed by brine (250 mL) and the mixture adjusted to pH 3-4 with 1N HCl. Extraction with ethyl acetate (3×250 mL) followed by concentration at reduced pressure affords an oil which is chromatographed over silica gel to give 3.85 g of 2-chloro-1-pyrimidin-2-ylethanol as a pale yellow oil. Physical characteristics: IR (liq.) 3427, 2404, 2346, 2196, 1980, 1568, 1439, 1425, 1390, 1183, 1111, 1087, 812, 658, 636 cm⁻¹.

In a pressure bottle is placed 2-chloro-1-pyrimidin-2-ylethanol (3.525 g), sodium iodide (0.344 g) and a 2M solution of methylamine in methanol (160 ml,). The bottle is sealed and heated at 62° C. for 17 h. The solvent is evaporated and the residue is stirred with 10% MeOH—CHCl₃. The mixture is filtered and concentrated to give an oil that is chromatographed over silica gel eluting with 5-10% MeOH—CH₂Cl₂ containing 1% triethylamine. The product containing fractions are combined and concentrated to give the title compound as an amber oil (1.625 g). Physical characteristics: ¹H NMR (400 MHz, CDCl₃) δ 2.53, 3.03, 3.21, 3.66, 5.03, 7.26, 8.77; HRMS (FAB) found 263.0636 (M+H)⁺.

(13) Preparation of R-2-(1-hydroxy-2-N-methylamino-ethyl)-pyridine.

2-Acetylpyridine (50 g) is placed in a 2L 1N round bottom flask and anhydrous CH₂Cl₂ (Alrdich Sure Seal®, 0.65 L) is added, followed by the addition of i-Pr₂NEt (160.27 g). The flask is equipped with a 125 mL pressure equalized dropping funnel, and the mixture is placed under nitrogen and cooled in an ice-water bath. To the chilled ketone/amine mixture is added TIPSOTf (139.7 g) over 1.5 hours. The mixture is allowed to warm to room temperature overnight. The reaction mixture is concentrated in vacuo on a rotary evaporator (T≦25° C.) to give a yellow oil and a white solid. The flask contents are transferred to a 2L separatory funnel with ether (1.2L) resulting in the formation of additional white solid material and the mixture is washed with saturated aq. NaHCO₃ (2×0.65L). The organic phase is separated, dried over Na₂SO₄, then is concentrated in vacuo to furnish the crude 2-[1-Tri-isopropylsilyloxy-vinyl]-pyridine (131.5 g) as a yellow-orange oil. This crude material is not further purified, but is immediately carried to the next step. ¹H-NMR (400 MHz, CDCl₃) δ =8.57, 7.71, 7.21, 5.65, 4.58, 1.36, 1.15.

Crude 2-[1-Tri-isopropylsilyloxy-vinyl]-pyridine (131.5 g) is placed in a 2L, 1N round bottom flask and dissolved in anhydrous THF (Aldrich Sure Seal, 0.6L). The flask is equipped with a reflux condenser and the apparatus is placed under nitrogen. NCS (60.66 g) is added and the mixture is heated to reflux and maintained at reflux for 2 hours. The reaction mixture is cooled to room temperature, poured into a 4L separatory funnel containing ether (1.5L), and is washed with saturated aq. NaHCO₃ (2×0.7L). The organic phase is separated, dried (Na₂SO₄), and concentrated in vacuo to afford crude 2-[1-tri-isopropylsilyloxy-2-chloro-vinyl]-pyridine (117.5 g) as a yellow-orange oil. The crude material is not further purified, but is immediately carried into the next step. ¹H-NMR (400 MHz, CDCl₃) δ 8.53, 7.71, 7.52, 7.22, 6.58, 1.21, 1.13.

Crude 2-[1-tri-isopropylsilyloxy-2-chloro-vinyl]-pyridine (117.3 g) is placed in a 4L plastic bottle and is dissolved in acetonitrile (0.4 L). To the stirring solution is added 48% aqueous HF (170 mL) and the progress of the reaction is monitored by reverse phase analytical HPLC. After ˜2 hours the reaction is judged to be complete, and the pH of the solution is carefully adjusted to ca. 8 with saturated aq. NaHCO₃. The mixture is poured into a separatory funnel containing CH₂Cl₂ (1.5L). The organic phase is removed and the aq. Layer is extracted with CH₂Cl₂ (2×1.0L). The combined organic layers are dried (Na₂SO₄), and concentration in vacuo to give the crude 2-chloroacetyl pyridine (49.5 g) as a tan solid after cooling. The crude material is used as is in the asymmetric reduction. ¹H-NMR (400 MHz, CDCl₃) δ 8.66, 8.09, 7.88, 7.54, 5.12.

[RuCl₂(η⁶-p-cymene)]₂ (0.84 g), Et₃N (0.67 g), and (1R, 2R)-N-p-toluenesulfonyl-1,2-diphenylethylenediamine (1.0 g) are combined in a 500 mL 1N round bottom flask. i-PrOH is added, a reflux condenser is attached and the mixture is warmed under reflux, and maintained, for 1 hour. The mixture is cooled to room temperature and is concentrated in vacuo (rotovapor followed by vacuum pump) to furnish the catalyst as a brown powdery solid. To the catalyst is added anhydrous DMF (Aldrich Sure Seal, 225 mL), followed in order by 2-chloroacetylpyridine (23.88 g) and HCOOH/Et₃N (5:2, Fluka, 55 mL). Reaction progress is monitored by reverse phase analytical HPLC, and after 65 minutes of stirring, the starting material had been consumed. Quench the reaction by adding MeOH (25 mL), stir 5 minutes and then the solvent is removed in vacuo (cold finger rotovapor, vacuum pump) to give a red-black viscous oil. The crude material is taken up in Et₂O/CH₂Cl₂ (4:1, 1.25L), placed in a 3L separatory funnel, washed with saturated aq. NaHCO₃ (1.0L), brine (1.0L), and dried (Na₂SO₄). Filtration and concentration in vacuo afforded the crude product as a red-orange oil which is purified by chromatography on a column of silica gel (70 mm OD, 250 g 230-400 mesh, packed hexanes; compound applied in CH₂Cl₂/hexanes 60:40; eluted with hexanes/Et₂O (75:25; 65:35; 55:45) using the flash technique. Product fractions are combined to afford 16.41 g of the target S-2-(1-hydroxy-2-chloroethyl)-pyridine as pale yellow solid. mp 49-50° C.; ¹H-NMR (400 MHz, CDCl₃): δ 8.60, 7.77, 7.58, 7.30, 5.00, 4.20, 3.85; Anal. Found: C, 53.27; H, 5.19; N, 8.81, Cl, 22.29; Specific Rotation [α]^{D}₂₅=62 (c 0.94, methanol).

S-2-(1-hydroxy-2-chloroethyl)-pyridine (6.0 g) and NaI (0.57 g) are combined in a 500 mL, plastic coated, thick walled bottle and are covered with 2M MeNH₂ in MeOH (0.19L). The Teflon stopper is wrapped in Teflon tape and the bottle is sealed. Stirring is started, and the bottle is immersed in a 60° C. oil bath for 16 hours. The yellow-brown mixture is cooled to room temperature. Concentration in vacuo affords the crude product as a yellow oil, which is treated with CH₂Cl₂-THF (0.25L, 10:90) to give a yellow solution and a white precipitate. The precipitate is removed by filtration, is rinsed with CH₂Cl₂-THF (10:90) and the combined filtrated are concentrated in vacuo to give a yellow-brown oil. The crude product is purified by chromatography on a column of silica gel (70 mm OD, 250 g, 230-400 mesh; packed with CH₂Cl₂—MeOH 90:10; eluted with CH₂Cl₂—MeOH 90:10, CH₂Cl₂—MeOH—NH₄OH 89:10:1) using the flash technique. Product fractions are combined to provide 3.18 g of aminoethanol R-2-(1-hydroxy-2-N-methylamino-ethyl)-pyridine as an amber oil. ¹H-NMR (400 MHz, DMSO-d₆): δ =8.49, 7.79, 7.52, 7.25, 4.75, 2.90, 2.67, 2.32; HRMS (FAB): Found 153.1009; Specific Rotation [α]^{D}₂₅=49 (c 0.36, CH₂Cl₂).

(14) Preparation of N-Methyl R-1-(2-furyl)-2-aminoethanol.

2-Acetylfuran (50 g) is placed in a 2L 1N round bottom flask and anhydrous CH₂Cl₂ (Alrdich Sure Seal, 0.70L) is added, followed by the addition of i-Pr₂NEt (176 g). The flask is equipped with a 125 mL pressure equalized dropping funnel, and the mixture is placed under nitrogen and cooled in an ice-water bath. To the chilled ketone/amine mixture is added TIPSOTf (153.2 g) over 1.5 hours. The mixture is allowed to warm to room temperature overnight. The reaction mixture is concentrated in vacuo on a rotary evaporator (T<25° C.) to give a yellow oil and a white solid. The flask contents are transferred to a 2L separatory funnel with ether (1.2L) resulting in the formation of additional white solid material and the mixture is washed with saturated aq. NaHCO₃ (2×0.70L). The organic phase is separated, dried over Na₂SO₄, then is concentrated in vacuo to furnish the crude 2-[1-tri-isopropylsilyloxy-vinyl]-furan (118.3 g) as a yellow-orange oil. This crude material is not further purified, but is immediately carried to the next step. ¹H-NMR (400 MHz, CDCl₃) δ 7.36, 6.49, 6.40, 4.86, 4.37, 1.32, 1.14.

Crude 2-[1-tri-isopropylsilyloxy-vinyl]-furan (116.3 gl) is placed in a 2L, 1N round bottom flask and dissolved in anhydrous THF (Aldrich Sure Seal, 0.6L). The flask is placed under nitrogen, cooled in a −10° C. bath, then NCS (64.11 g) is added and the mixture is stirred for 1 hour, after which time the reaction is judged to be complete by analytical reverse phase HPLC. The reaction mixture is warmed to room temperature, poured into a 4L separatory funnel containing ether (1.5L), and is washed with saturated aq. NaHCO₃ (2×0.7L). The organic phase is separated, dried (Na₂SO₄), and concentrated in vacuo to afford 2-[1-tri-isopropylsilyloxy-2-chloro-vinyl]-furan (129.9 g) as a yellow-orange oil. The crude material is not further purified, but is immediately carried into the next step. ¹H-NMR (400 MHz, CDCl₃) δ 7.36, 6.43, 6.40, 5.95, 1.30, 1.11.

2-[1-Tri-isopropylsilyloxy-2-chloro-vinyl]-furan (129.9 g) is placed in a 4L plastic bottle and is dissolved in acetonitrile (0.6L). To the stirring solution is added 48% aqueous HF (65 mL) and the progress of the reaction is monitored by reverse phase analytical HPLC. After. Ca. 2 hours the reaction is judged to be complete, and the pH of the solution is carefully adjusted to ca. 7 with saturated aq. NaHCO₃. The mixture is poured into a separatory funnel containing CH₂Cl₂ (1.5L). The organic phase is removed and the aq. layer is extracted with CH₂Cl₂ (2×1.0L). The combined organic layers are dried (Na₂SO₄), and concentration in vacuo afforded the crude 2-chloroacetyl furan (41.9 g) as a yellow oil. The crude material is used as is in the asymmetric reduction. ¹H-NMR (400 MHz, CDCl₃): δ 7.58, 7.33, 6.59, 4.57; MS (ES+): 145.4 (M+H⁺).

[RuCl₂(η⁶-p-cymene)]₂ (0.99 g), Et₃N (0.67 g), and (1R, 2R)-N-p-toluenesulfonyl-1,2-diphenylethylenediamine (1.18 g) are combined in a 500 mL 1N round bottom flask. i-PrOH (25 mL) and Et₃N (0.67 g) are added, a reflux condenser is attached and the mixture is warmed under reflux, and maintained, for 1 hour. Cool to room temperature and concentrate in vacuo (rotovapor) to furnish the catalyst as an orange-brown powdery solid. To the catalyst is added anhydrous DMF (Aldrich Sure Seal, 250 mL), followed in order by 2-chloroacetylfuran (20.6 g) and HCOOH/Et₃N (5:2, Fluka, 51 mL). Reaction progress is monitored by reverse phase analytical HPLC, and after 65 minutes of stirring, the starting material had been consumed. Quench the reaction by adding MeOH (25 mL), stir 5 minutes and then the reaction mixture is poured into ice-water (1L) and the aqueous phase is saturated with salt. The mixture is transferred to a 2L separatory funnel with ether (500 mL), shaken, and the organic phase is removed. The aqueous layer is extracted with ether (3×250 mL) and the combined organic layers are washed with saturated aq. NaHCO₃ (0.5L), brine (4×250mL), and dried (Na₂SO₄). Filtration and concentration in vacuo affords the crude product as a red-orange oil (20.5 g) that is triturated with ether/pentane (10:90, 4×100 mL). The combined triturates are concentrated in vacuo carefully (the halohydrin is volatile, hence the choice of ether/pentane as triturant and no removal of DMF in vacuo) to furnish the desired halohydrin S-1-(2-furyl)-2-chloroethanol (15.97 g). ¹H-NMR (400 MHz, CDCl₃) δ 7.41, 6.37, 4.95, 3.85, 2.58; HRMS (EI) Found 146.0136; Specific Rotation [α]^{D}₂₅=17 (c 0.97, methanol).

To (S)-1-(2-furyl)-2-chloroethanol (5.0 g) in dry CH₂Cl₂ (Aldrich Sure Seal®, 75 mL), cooled in an ice-water bath under nitrogen, is added Et₃N (1.38 g). Stir 5 minutes, then methyl isocyanate (3.32 g) is added via syringe over 2 minutes. Allow the ice to melt and the mixture to warm toward room temperature while monitoring the reaction by HPLC. Allow to stir overnight and the mixture is poured into Et₂O (0.3L) and brine (0.3L). The organic phase is reserved, the aq. layer is extracted with Et₂O (2×0.2L); the combined organic phases are washed with brine (0.4L), and dried (Na₂SO₄). Concentration in vacuo afforded the crude carbamate as a brown, viscous oil, which is purified by chromatography (Biotage® 40 g column, EtOAc/hexanes 10:90, EtOAc/hexanes 20:80). Product fractions afford 4.56 g of S-1-(2-furyl)-2-chloroethanol-N-methylcarbamate as a clear, pale yellow oil which solidified to an ivory solid upon cooling. mp 26-27° C.; ¹H-NMR (400 MHz, CDCl₃) δ 7.43, 6.45, 6.39, 5.97, 4.79, 3.89, 2.82; ¹³C-NMR (100 MHz, CDCl₃): δ 156.2, 150.3, 143.3, 110.8, 109.9, 69.1, 44.0, 28.0; KF Moisture: 0.13%; Anal. Found: C, 46.99; H, 4.89; N, 6.85; Cl, 17.31; Specific Rotation [α]^{D}₂₅=94 (c 1.02, CH₂Cl₂).

The crude carbamate is dissolved in dry THF (0.2L, Aldrich Sure Seal®) and the solution is cooled in an ice-water bath under nitrogen. To the chilled carbamate solution is added KOtBu (1.0M in THF, 97 mL) over 15 minutes. The mixture is allowed to stir after the addition is complete and HPLC analysis suggests that the reaction is complete within 15 minutes. The mixture is cast into Et₂O (1.25L) and brine (1.0L) containing 1N aq. HCL (50 mL). The organic phase is separated, the aqueous layer is extracted with Et₂O (1.0L). The combined organic phases are washed with saturated aq. NaHCO₃ (1.0L) and dried (Na₂SO₄). Concentration in vacuo afforded the crude oxazolidinone as a red-black oil which is triturated with pentane-Et₂O (2:1; 3×0.2L). The pentane-Et₂O aliquots are concentrated in vacuo to give a red solid which is purified by chromatography on a 120 g Biotage® column (introduced as a solution in CH₂Cl₂, eluted with EtOAc/hexanes, 35:65; EtOAc/hexanes 50:50). Product fractions are combined to afford 8.75 g of 5R-3-methyl-5-(2-furyl)-2-oxazoldinone as a pale yellow oil, which solidified to furnish an ivory solid upon cooling. mp 54-55° C.; ¹H-NMR (400 MHz, CDCl₃) δ 7.47, 6.49, 6.41, 5.46, 3.78, 2.97; ¹³C-NMR (100 MHz, CDCl₃) δ 155.9, 148.1, 142.1, 109.0, 108.4, 65.9, 48.8, 29.4; KF Moisture: 0.07%; Anal. Found: C, 57.46; H, 5.39; N, 8.36; Specific Rotation [α]^{D}₂₅=−106 (c 1.01, CH₂Cl₂).

To 5R-3-methyl-5-(2-furyl)-2-oxazoldinone (8.0 g) in a 500 mL 1N RB flask is added 1N aq. KOH (240 mL). The flask is equipped with a reflux condenser, placed under nitrogen, then is immersed in a preheated (50° C.) oil bath. The mixture is allowed to stir and the 5R-3-methyl-5-(2-furyl)-2-oxazoldinone suspension slowly affords a clear solution. After stirring for 3 hours at 50° C., HPLC analysis indicated that the reaction is complete. The mixture is cooled to room temperature and is cast into a separatory funnel, the flask is rinsed into the separatory funnel with Et₂O/CH₂Cl₂ (95:5, 0.5L) and the aq. layer is saturated with salt. The organic phase is removed, the aq. phase is extracted with Et₂O/CH₂Cl₂ (95:5, 2×0.5L) and the combined organic phases are dried (Na₂SO₄). Concentration in vacuo affords N-methyl R-1-(2-furyl)-2-aminoethanol (6.50 g) as a pale orange oil which solidifies at freezer (−20° C.) temperatures. ¹H-NMR (400 MHz, DMSO-d₆) δ 7.55, 6.37, 6.25, 4.59, 2.70, 2.25; ¹³C-NMR (100 MHz, DMSO-d₆) δ 157.3, 141.9, 110.5, 105.9, 65.5, 56.5, 36.5; KF Moisture: 0.83%; Anal. Found: C, 59.90; H, 7.83; N, 9.68; Specific Rotation [α]^{D}₂₅=32 (c 0.96, EtOH).

(15) Preparation of S-3-(1-hydroxy-2-N-methylamino-ethyl)-pyridine.

As described for the preparation of N-methyl R-1-(2-furyl)-2-aminoethanol, 3-chloroacetylpyridine (0.075 g) (Chem. Ber. 1951, 84, 147-149) is reduced with ([RuCl₂(η⁶-p-cymene)]₂ (3 mg), Et₃N (3 μL), and (1R, 2R)-N-p-toluenesulfonyl-1,2-diphenylethylenediamine (3 mg), in DMF (2 mL) containing HCOOH/Et₃N (5:2, 0.18 mL) to give S-2-(1-hydroxy-2-chloroethyl)-pyridine (0.055 g) as a pale yellow solid. ¹H NMR (300 MHz, CDCl₃) δ 8.53, 7.79, 7.32, 4.97, 3.88, 3.72; Chiral HPLC Analysis (Chiracel OJ): 99:1.

As described for the preparation of N-methyl R-1-(2-furyl)-2-aminoethanol, S-2-(1-hydroxy-2-chloroethyl)-pyridine is treated with methyl isocyanate (3.21 mL) and Et₃N (1.76 mL) in anhydrous CH₂Cl₂ (75 mL, Aldrich Sure Seal®) to give 5R-3-methyl-5-(3-pyridyl)-2-oxazoldinone (6.05 g) as a white solid. Mp 79-80° C.; ¹H-NMR (400 MHz, CDCl₃) δ 8.65, 8.59, 7.69, 7.32, 5.93, 5.16, 3.79, 2.81; Specific Rotation [α]²⁵_{D}=33° (c 0.96, chloroform); Anal. Calcd for C₉H₁₁ClN₂O₂: C, 50.36; H, 5.16; N, 13.05; Cl, 16.52; Found: C, 50.29; H, 5.18; N, 12.96; Chiral HPLC Analysis (Chiracel OJ): 98.3:1.7.

As described for the preparation of N-methyl R-1-(2-furyl)-2-aminoethanol, 5R-3-methyl-5-(3-pyridyl)-2-oxazoldinone (4.19 g) is treated with 1N aq. KOH (0.12L) and THF (50 mL) to give S-3-(1-hydroxy-2-N-methylamino-ethyl)-pyridine (2.36 g) as a pale yellow amorphous solid. IR (diffuse reflectance) 3303, 3295, 3087, 3053, 3035, 2977, 2889, 2840, 2793, 2311, 2265, 2178, 2114, 2092, 713 cm⁻¹; HRMS (ESI) found 153.1017 for C₈H₁₂N₂O+H₁; Specific Rotation [α]²⁵_{D}=70° (c 1.03, methylene chloride); Chiral HPLC Analysis (Chiracel OJ): 98.7:1.3.

(16) Preparation of S-3-(1-hydroxy-2-N-methylamino-ethyl)-thiophene.

As described for the preparation of N-methyl R-1-(2-furyl)-2-aminoethanol, 3-acetylthiophene (50 g) in anhydrous CH₂Cl₂ (Alrdich Sure Seal®, 600 mL) is treated with i-Pr₂NEt (1.18 mol), TIPSOTf (117.2 mL) to give 3-[1-tri-isopropylsilyloxy-vinyl]-thiophene (117 g) as a brown oil. This crude material is not further purified, but is immediately carried to the next step. ¹H-NMR (300 MHz, CDCl₃) δ 7.26, 7.20, 6.99, 4.79, 4.33, 1.21-1.41, 1.20.

As described for the preparation of N-methyl R-1-(2-furyl)-2-aminoethanol, 3-[1-tri-isopropylsilyloxy-vinyl]-thiophene (117 g) is treated with NCS (52.87 g) to give 138 g of crude 3-[1-tri-isopropylsilyloxy-2-chloro-vinyl]-thiophene. ¹H-NMR (400 MHz, CDCl₃) δ 7.00-7.40, 5.80-5.95, 1.00-1.40.

As described for the preparation of N-methyl R-1-(2-furyl)-2-aminoethanol, 3-[1-tri-isopropylsilyloxy-2-chloro-vinyl]-thiophene (138 g) is treated with 48% aq. HF (159 mL) in acetonitrile (0.5L) to give 3-chloroacetyl-thiophene (54.6 g) as an amorphous pale yellow solid. ¹H-NMR (400 MHz, CDCl₃) δ 8.18, 7.60, 7.40, 4.61.

As described for the preparation of N-methyl R-1-(2-furyl)-2-aminoethanol, 3-chloroacetyl-thiophene (43 g) is reduced ([RuCl₂(η⁶-p-cymene)]₂ (1.64 g), Et₃N (1.4 mL), (1R, 2R)-N-p-toluenesulfonyl-1,2-diphenylethylenediamine (1.96 g), i-propanol (30 mL), anhydrous DMF (Aldrich Sure Seal®, 200 mL), HCOOH/Et₃N (5:2, Fluka, 97 mL)) to give S-1-(3-thienyl)-2-chloroethanol (27.4 g) as a pale yellow oil. ¹H-NMR (400 MHz, CDCl₃) δ 7.28-7.40, 7.11, 5.02, 3.82, 3.71; HRMS (EI) found 161.9905 for C₆H₇CLOS; Specific Rotation [α]²⁵_{D}=40° (c 1.01, methylene chloride); Chiral HPLC Analysis (Chiracel OJ): 98.8:1.2.

As described for the preparation of N-methyl R-1-(2-furyl)-2-aminoethanol, S-1-(3-thienyl)-2-chloroethanol (5.00 g) is treated with Et₃N (1.24 g) and methyl isocyanate (2.98 g) in CH₂Cl₂ (35 mL) to give S-1-(3-thienyl)-2-chloroethanol-N-methylcarbamate 5.91 g as a clear, colorless oil. ¹H-NMR (300 MHz, CDCl₃) δ 7.30-7.40, 7.09, 6.03, 4.82, 3.82), 2.82; Specific Rotation [α]²⁵_{D}=57° (c 0.73, methylene chloride); Chiral HPLC Analysis (Chiracel OJ): 98.5:1.5.

As described for the preparation of N-methyl R-1-(2-furyl)-2-aminoethanol, S-1-(3-thienyl)-2-chloroethanol-N-methylcarbamate (5.39 g, in THF (30 mL), is treated with NaH (1.19 g) to afford 5R-3-methyl-5-(3-thienyl)-2-oxazoldinone (3.80 g) as a pale yellow solid. mp 68-69° C.; IR (diffuse reflectance) 3096, 2483, 2408, 2350, 2328, 2253, 1755, 1733, 1501, 1439, 1408, 1264, 1249, 1137, 1030 cm⁻¹;HRMS (ESI) found 184.0432 for C₈H₉NO₂S+H₁; Specific Rotation [α]²⁵_{D}=−14° (c 1.05, chloroform); Anal. Calcd for C₈H₉NO₂S: C, 52.44; H, 4.95; N, 7.64; S, 17.50. Found: C, 52.38; H, 5.05; N, 7.60; S, 17.33; Chiral HPLC Analysis (Chiracel OJ): 98.4:1.6.

As described for the preparation of N-methyl R-1-(2-furyl)-2-aminoethanol, 5S-3-methyl-5-(3-thienyl)-2-oxazoldinone (3.35 g) is treated with 1N aq. KOH (95 mL) to provide S-3-(1-hydroxy-2-N-methylamino-ethyl)-thiophene (2.79 g) as a pale yellow oil. ¹H-NMR (300 MHz, CDCl₃) δ 7.31, 7.24, 7.07, 4.86, 2.60-3.00, 2.47; IR (liq.) 3315, 3102, 2972, 2941, 2890, 2857, 2800, 1996, 1473, 1451, 1066, 853, 836, 787, 652 cm⁻¹; HRMS (ESI) found 158.0628 for C₇H₁₁NOS+H₁; Specific Rotation [α]²⁵_{D}=48° (c 0.86, chloroform); Chiral HPLC Analysis (Chiracel OJ):>99:1.

(17) Preparation of R-2-(1-hydroxy-2-N-methylamino-ethyl)-pyrazine.

As described for the preparation of N-methyl R-1-(2-furyl)-2-aminoethanol, 2-acetylpyrazine (53.9 g) in anhydrous CH₂Cl₂ (Alrdich Sure Seal®, 700 mL) is treated with i-Pr₂NEt (171.0 g), TIPSOTf (148.6 g) to give 2-[1-tri-isopropylsilyloxy-vinyl]-pyrazine (132.9 g) as a brown oil. This crude material is not further purified, but is immediately carried to the next step. ¹H NMR (300 MHz, CDCl₃) δ 8.97, 8.49, 5.66, 4.65, 1.36, 1.14; ¹³C NMR (100 MHz, CDCl₃) δ 153.0, 150.0,143.8,143.5, 141.1, 94.1, 18.1, 12.7; HRMS (FAB) found 279.1891 for C₁₅H₂₆N₂OSi+H₁.

As described for the preparation of N-methyl R-1-(2-furyl)-2-aminoethanol, 2-[1-tri-isopropylsilyloxy-vinyl]-pyrazine (132.9 g) in anhydrous THF (Aldrich Sure Seal, 640 mL) is chlorinated with NCS (64.78 g) to give 2-[1-tri-isopropylsilyloxy-2-chloro-vinyl]-pyrazine (169.45 g) as a brown oil. The crude material is not further purified, but is immediately carried into the next step. ¹H NMR (400 MHz, CDCl₃) δ 8.82, 8.51, 8.49, 6.62, 1.33, 1.13; HRMS (FAB) found 313.1511 for C₁₅H₂₅ClN₂OSi+H₁.

As described for the preparation of N-methyl R-1-(2-furyl)-2-aminoethanol, 2-[1-tri-isopropylsilyloxy-2-chloro-vinyl]-pyrazine (169.45 g) is placed in a 4 L plastic bottle and dissolved in acetonitrile (470 mL) and treated with 48% aqueous HF (73.54 mL) to give 2-chloroacetylpyrazine (60.1 g) as a very light yellow solid. mp 82.6-83.8° C. (dec.); IR (diffuse reflectance) 2944, 1716, 1400, 1390, 1318, 1226, 1170, 1163, 1053, 1019, 1000, 851, 807, 790, 685 cm⁻¹; ¹H NMR (300 MHz, CDCl₃) δ 9.23, 8.80, 8.64, 5.01; ¹³C NMR (75 MHz, CDCl₃) δ 191.4, 148.4, 145.6, 143.5, 143.3, 46.4.

As described for the preparation of N-methyl R-1-(2-furyl)-2-aminoethanol, 2-chloroacetylpyrazine (60.1 g) is reduced ([RuCl₂(η⁶-p-cymene)]₂ (0.980 g), Et₃N (0.664 g), (1R, 2R)-N-p-toluenesulfonyl-1,2-diphenylethylenediamine (1.17 g) i-propanol (24.7 mL), anhydrous DMF (Aldrich Sure Seal®, 247 mL), HCOOH/Et₃N (5:2, Fluka, 57.3 mL)) to give (1S)-2-chloro-1-pyrazin-2-ylethanol (22.5 g) as a brown oil. IR (liq.) 3275, 3064, 2959, 2869, 1474, 1405, 1308, 1154, 1091, 1059, 1019, 856, 775, 663, 649 cm⁻¹; ¹H NMR (400 MHz, CDCl₃) δ 8.80, 8.56, 5.07, 3.93, 3.87; ¹³C NMR (100 MHz, CDCl₃) δ 154.2, 144.2, 143.5, 143.3, 71.7, 48.9; HRMS (FAB) calcd for C₆H₇ClN₂O+H₁ 159.0325, found 159.0323; Specific Rotation [α]_{D}²⁵+40 (c 0.61, ethanol); Anal. Found: C, 45.23; H, 4.60; N, 17.38.

As described for the preparation of R-2-(1-hydroxy-2-N-methylamino-ethyl)-pyridine, (1S)-2-chloro-1-pyrazin-2-ylethanol (11.8 g), NaI (1.12 g) and 2M MeNH₂ in MeOH (370 mL) affords of R-2-(1-hydroxy-2-N-methylamino-ethyl)-pyrazine (8.18 g) as a light orange liquid that solidified on standing. Mp 78-81° C.; ¹H NMR (400 MHz, CD₃OD) δ 8.79, 8.58, 8.53, 5.00, 3.15, 3.05, 2.55; ¹³C NMR (75 MHz, CD₃OD) δ 159.1, 145.1, 144.6, 143.9, 71.5, 57.5, 35.6; HRMS (FAB) found 154.0973 for C₇H₁₁N₃O+H₁; Specific Rotation [α]_{D}²⁵+58 (c 1.02, methanol).

Carbonyldiimidazole (4.26 g) is dissolved in dichloromethane (80 mL). To this solution is slowly added, via cannula addition, R-2-(i-hydroxy-2-N-methylamino-ethyl)-pyrazine (3.66 g) dissolved in dichloromethane (60 mL). The reaction is stirred at room temperature for 16 h. The solvent in vacuo and purification is accomplished by silica gel column chromatography (98:2 dichloromethane-methanol, sample and silica gel loaded in dichloromethane). Any uncyclized carbamate collected off the column is dissolved in methanol, to which is added a catalytic amount of 1 M NaOH and the solution is refluxed until completely cyclized. This freshly cyclized material is then purified by column chromatography as above. The combined pure oxazolidinone fractions are then concentrated in vacuo to provide (5R)-3-methyl-5-pyrazin-2-yl-1,3-oxazolidin-2-one (3.77 g) as a white solid. This material is upgraded by chiral preparative HPLC to give material with >95% ee. Mp 113.5-114.1° C.; ¹H NMR (400 MHz, CDCl₃) δ 8.84, 8.62, 8.58, 5.62, 4.02, 3.80, 2.94; ¹³C NMR (100 MHz, CDCl₃) δ 157.4, 153.2, 144.8, 144.1, 142.7, 72.2, 51.6, 31.1; HRMS (FAB) found 180.0781 for C₈H₉N₃O₂+H₁; Specific Rotation [α]_{D}²⁵+20 (c 0.95, methylene chloride); Anal. Found: C, 53.38; H, 5.03; N, 23.35.

As described for the preparation of N-methyl R-1-(2-furyl)-2-aminoethanol, (5R)-3-methyl-5-pyrazin-2-yl-1,3-oxazolidin-2-one (1.51 g) is treated with 1N aq. KOH (42.1 mL) to give R-2-(1-hydroxy-2-N-methylamino-ethyl)-pyrazine (1.02 gl) as a white solid. Mp 84-85° C.; Specific Rotation [α]_{D}²⁵+66 (c 0.94, methanol).

(18) Preparation of R-2-(1-hydroxy-2-N-methylamino-ethyl)-thiazole.

As described for the preparation of N-methyl R-1-(2-furyl)-2-aminoethanol, 2-acetyl thiazole (25 g) is treated with (i-Pr)₃SiOTf (66.26 g) and (i-Pr)₂NEt (76.22 g) in CH₂Cl₂ (0.35L) to give 2-[1-tri-isopropylsilyloxy-vinyl]-thiazole (59.45 g) as a golden yellow liquid. ¹H-NMR (400 MHz, CDCl₃) δ 7.80, 7.32, 5.50, 4.52, 1.35, 1.14-1.20.

As described for the preparation of N-methyl R-1-(2-furyl)-2-aminoethanol, 2-[1-tri-isopropylsilyloxy-vinyl]-thiazole (59.45 g) is treated with NCS (29.37 g) in THF (0.35L) to give 2-[1-tri-isopropylsilyloxy-2-chloro-vinyl]-thiazole (69.61 g) as a yellow-orange semi-solid. ¹H-NMR (400 MHz, CDCl₃) δ 7.77, 7.32, 6.57, 1.37, 1.10-1.22.

As described for the preparation of N-methyl R-1-(2-furyl)-2-aminoethanol, 2-[1-tri-isopropylsilyloxy-2-chloro-vinyl]-thiazole (69.61 g) is treated with 48% aq. HF in CH₃CN (0.3L) to give 2-chloroacetylthiazole (32.95 g) as a light brown liquid which solidified to a tan solid upon cooling. ¹H-NMR (400 MHz, CDCl₃) δ 8.06, 7.79, 5.00; HRMS (ESI) found 161.9779 for C₅H₄ClNOS+H; Anal. Found: C, 37.17; H, 2.51; N, 8.62.

As described for the preparation of N-methyl R-1-(2-furyl)-2-aminoethanol, 2-chloroacetylthiazole (24.7 g) is reduced ([RuCl₂(η⁶-p-cymene)]₂ (0.84 g), Et₃N (0.78 mL), (1R, 2R)-N-p-toluenesulfonyl-1,2-diphenylethylenediamine (1.00 g) i-propanol (25 mL), anhydrous DMF (Aldrich Sure Seal®, 250 mL), HCOOH/Et₃N (5:2, Fluka, 55 mL)) to give S-1-(2-thiazolyl)-2-chloroethanol (13.9 g) as a white amorphous solid. ¹H-NMR (400 MHz, CDCl₃) δ 7.78, 7.36, 5.24, 4.06, 3.88; % Water (KF): 0.06; Specific Rotation [α]²⁵_{D}=32° (c 0.83, methylene chloride); Anal Found: C, 36.68; H, 3.58; N, 8.53; Cl, 21.37; S, 19.10; Chiral HPLC Analysis (Chiracel OJ):>99:1.

As described for the preparation of R-2-(1-hydroxy-2-N-methylamino-ethyl)-pyridine, S-1-(2-thiazolyl)-2-chloroethanol (6.02 g) is treated with NaI (0.57 g) and MeNH₂ (2.0M in MeOH, 0.2L) to give R-2-(1-hydroxy-2-N-methylamino-ethyl)-thiazole (2.08 g) as a pale yellow oil. ¹H-NMR (400 MHz, CDCl₃) δ 7.71, 7.31, 5.68, 5.36, 3.34, 3.15, 2.64; HRMS (ESI) found 159.0582 for C₆H₁₀N₂OS+H₁; Specific Rotation [α]²⁵_{D}=31° (c 1.02, DMSO); Chiral HPLC Analysis (Chiracel OJ):>99:1.

(19) Preparation of R-2-(1-hydroxy-2-N-methylamino-ethyl)-thiophene.

As described for the preparation of N-methyl R-1-(2-furyl)-2-aminoethanol, 2-acetylthiophene (50 g) is treated with (i-Pr)₃SiOTf (133.6 g) and (i-Pr)₂NEt (153.6 g) in CH₂Cl₂ (0.7L) to give 2-[1-tri-isopropylsilyloxy-vinyl]-thiophene (111.9 g) as a golden yellow liquid. ¹H-NMR (300 MHz, CDCl₃) δ 7.26, 7.22, 6.99, 4.79, 4.33, 1.32, 1.10-1.22; MS (ES+): 283.2 (M+H).

As described for the preparation of N-methyl R-1-(2-furyl)-2-aminoethanol, 2-[1-tri-isopropylsilyloxy-vinyl]-thiophene (111.19 g) is treated with NCS (58.2 g) in THF (0.6L) to afford 2-[1-tri-isopropylsilyloxy-2-chloro-vinyl]-thiophene (140 g) as a viscous orange oil. ¹H-NMR (300 MHz, CDCl₃) δ 6.85-7.40, 5.70-5.85, 0.95-1.35.

As described for the preparation of N-methyl R-1-(2-furyl)-2-aminoethanol, 2-[1-tri-isopropylsilyloxy-2-chloro-vinyl]-thiophene (140 g) in CH₃CN (0.4L) is treated with 48% aq. HF to give 2-chloroacetylthiophene (48 g) as a light brown solid. ¹H-NMR (400 MHz, CDCl₃) δ 7.81, 7.76, 7.19, 4.62.

As described for the preparation of N-methyl R-1-(2-furyl)-2-aminoethanol, 2-chloroacetylthiophene (26 g) is reduced ([RuCl₂(η⁶-p-cymene)]₂ (0.99 g), Et₃N (0.93 mL), (1R, 2R)-N-p-toluenesulfonyl-1,2-diphenylethylenediamine (1.18 g) i-propanol (25 mL), anhydrous DMF (Aldrich Sure Seal®, 250 mL), HCOOH/Et₃N (5:2, Fluka, 58 mL)) to give S-1-(2-thienyl)-2-chloroethanol (17.8 g) as a clear colorless liquid. ¹H NMR (400 MHz, CDCl₃) δ 7.30, 7.05, 7.01, 5.17, 3.76, 2.81; HRMS (EI) found 161.9908 for C₆H₇ClOS; Specific Rotation [α]²⁵_{D}=30 (c 0.90, methylene chloride); Chiral HPLC Analysis (Chiracel OJ): 99:1.

As described for the preparation of N-methyl R-1-(2-furyl)-2-aminoethanol, S-1-(2-thienyl)-2-chloroethanol (5.54 g) is treated with Et₃N (1.9 mL) and methyl isocyanate (3.36 g) in CH₂Cl₂ (75 mL) to give S-1-(2-thienyl)-2-chloroethanol-N-methylcarbamate 6.87 g as a clear, colorless oil. ¹H NMR (400 MHz, CDCl₃) δ 7.30, 7.11, 7.00, 6.17, 4.77, 3.82, 2.81; HRMS (FAB) found 220.0208 for C₈H₁₀ClNO₂S+H₁; % Water (KF): 0.25; Specific Rotation [α]²⁵_{D} =58 (c 0.97, methylene chloride); Anal. Found: C, 43.59; H, 4.39; N, 6.32; Chiral HPLC Analysis (Chiracel OJ): 98.3:1.7.

As described for the preparation of N-methyl R-1-(2-furyl)-2-aminoethanol, S-1-(2-thienyl)-2-chloroethanol-N-methylcarbamate (6.47 g) in THF (35 mL) is treated with NaH (2.33 g, 60% oil dispersion) to afford 5R-3-methyl-5-(2-thienyl)-2-oxazoldinone (4.94 g as an ivory amorphous solid. ¹H NMR (400 MHz, CDCl₃) δ 7.36, 7.14, 7.01, 5.70, 3.90, 3.61, 2.95; HRMS (ESI) found 184.0435 for C₈H₉NO₂S+H; Specific Rotation [α]²⁵_{D}=−94 (c 1.04, methylene chloride); (Chiracel OJ):>99:1.

As described for the preparation of N-methyl R-1-(2-furyl)-2-aminoethanol, 5R-3-methyl-5-(2-thienyl)-2-oxazoldinone (4.63 g) is treated with 1N aq. KOH (100 mL) in THF (50 mL) to provide R-2-(1-hydroxy-2-N-methylamino-ethyl)-thiophene (3.28 g) as an ivory solid. ¹H NMR (400 MHz, CDCl₃) δ 7.26, 6.98, 4.99, 2.89, 2.48; Specific Rotation [α]²⁵_{D}=26 (c 1.05, methylene chloride); Anal. Found: C, 53.31; H, 7.13; N, 8.84; Chiral HPLC Analysis (Chiracel OJ):>99:1.

(20) Preparation of (R)-1-(1-benzofuran-2-yl)-2-(methylamino)ethanol.

As described for the preparation of N-methyl R-1-(2-furyl)-2-aminoethanol, 2-acetyl benzofuran (32.03 g) is treated with (i-Pr)₃SiOTf (80 mL) and (i-Pr)₂NEt (104 mL) in CH₂Cl₂ (0.3L) to give 2-[1-tri-isopropylsilyloxy-vinyl]-benzofuran (72.52 g) as an orange oil. ¹H-NMR (400 MHz, CDCl₃) δ 7.58, 7.47, 7.30, 7.23, 5.18, 4.58, 1.23-1.42, 1.18.

As described for the preparation of N-methyl R-1-(2-furyl)-2-aminoethanol, 2-[1-tri-isopropylsilyloxy-vinyl]-benzofuran (79.52 g) is treated with NCS (29.4 g) in THF (0.4L) to afford 2-[1-tri-isopropylsilyloxy-2-chloro-vinyl]-benzofuran (79.52 g) as a viscous yellow oil.

As described for the preparation of N-methyl R-1-(2-furyl)-2-aminoethanol, 2-[1-tri-isopropylsilyloxy-2-chloro-vinyl]-benzofuran (79.52 g) is treated with 48% aq. HF (40 mL) in CH₃CN to give 2-chloroacetyl-benzofuran (23.32 g) as a powdery, tan solid. ¹H-NMR (400 MHz, CDCl₃) δ 7.76, 7.68, 7.61, 7.54, 7.37, 4.73; IR (diffuse reflectance) 2479, 2412, 2360, 2338, 2306, 1694, 1550, 1271, 1263, 1255, 1164, 1023, 752, 743, 716 cm⁻¹; Anal. Found: C, 61.92; H, 3.68.

As described for the preparation of N-methyl R-1-(2-furyl)-2-aminoethanol, 2-chloroacetyl benzofuran (10.0 g) is reduced ([RuCl₂(ι⁶-p-cymene)]₂ (0.57 g), Et₃N (0.47 mL), (1R, 2R)-N-p-toluenesulfonyl-1,2-diphenylethylenediamine (0.56 g) i-propanol (20 mL), anhydrous DMF (Aldrich Sure Seal®, 250 mL), HCOOR/Et₃N (5:2, Fluka, 58 mL)) to give S-1-(2-benzofuranyl)-2-chloroethanol (9.26 g) as a light brown oil. ¹H NMR (400 MHz, CDCl₃) δ 7.58, 7.48, 7.22-7.34, 6.79, 5.10, 3.98; IR (liq.) 3367, 1663, 1454, 1437, 1414, 1388, 1254, 1171, 1095, 1011, 881, 808, 775, 753, 664 cm⁻¹; HRMS (EI) found 196.0295 for C₁₀H₉ClO₂; Specific Rotation [α]²⁵_{D}=31 (c 1.05, chloroform); Chiral HPLC Analysis (Chiracel OJ): 98:2.

As described for the preparation of N-methyl R-1-(2-furyl)-2-aminoethanol, S-1-(2-benzofuranyl)-2-chloroethanol (8.45 g) is treated with methyl isocyanate (4.17 g) and Et₃N (1.74 g) in CH₂Cl₂ (50 mL) to give S-1-(2-benzofuranyl)-2-chloroethanol-N-methylcarbamate (9.75 g) as a white solid. Mp 77-78° C.; ¹H NMR (400 MHz, CDCl₃) δ 7.58, 7.49, 7.23-7.35, 6.83, 6.11, 4.58, 3.99, 2.85; IR (diffuse reflectance) 3374, 1699, 1535, 1251, 1134, 975, 924, 821, 814, 771, 748, 733, 676, 626, 613 cm⁻¹; Specific Rotation [α]²⁵_{D} =101 (c 0.85, chloroform); Anal. Found: C, 57.04; H, 4.77; N, 5.55; Cl, 13.53.

As described for the preparation of N-methyl R-1-(2-furyl)-2-aminoethanol, S-1-(2-benzofuranyl)-2-chloroethanol-N-methylcarbamate (9.67 g) in THF (0.1 L) is treated with KOtBu (38.5 mL, 1.0M in THF) to give 5R-3-methyl-5-(2-benzofuranyll)-2-oxazoldinone (5.05 g) as a tan solid. Mp 68-69°; ¹H NMR (400 MHz, CDCl₃) δ 7.59, 7.50, 7.35, 7.28, 6.83, 5.62, 3.87, 3.00; Specific Rotation [α]²⁵_{D}=−38 (c 0.95, chloroform); Anal. Found: C, 66.14; H, 5.07; N, 6.30; chiral HPLC Analysis (Chiracel OJ): 96:4.

As described for the preparation of N-methyl R-1-(2-furyl)-2-aminoethanol, 5R-3-methyl-5-(2-benzofuranyll)-2-oxazoldinone (4.89 g) and 1N KOH (10 ml) in EtOH (75 ml) affords 3.97 g of (R)-1-(1-benzofuran-2-yl)-2-(methylamino)ethanol as a tan solid. Mp 88-90° C.; ¹H NMR (400 MHz, CDCl₃) δ 7.56, 7.47, 7.20-7.31, 6.70, 4.92, 3.10,3.02, 2.51; HRMS (FAB) found 192.1026 for C₁₁H₁₃NO₂+H₁; Specific Rotation [α]²⁵_{D}=31° (c 1.05, chloroform); Anal. Found: C, 68.71; H, 6.98; N, 7.16; chiral HPLC Analysis (Chiracel OJ): 96:4.

(21) Preparation of (S)-1-(1-benzofuran-2-yl)-2-(methylamino)ethanol.

As described for the preparation of N-methyl R-1-(2-furyl)-2-aminoethanol, 2-chloroacetyl benzofuran (10.0 g) is reduced ([RuCl₂(η⁶-p-cymene)]₂ (0.57 g), Et₃N (0.47 mL), (1S, 2S)-N-p-toluenesulfonyl-1,2-diphenylethylenediamine (0.56 g), i-propanol (20 mL), anhydrous DMF (Aldrich Sure Seal®, 250 mL), HCOOH/Et₃N (5:2, Fluka, 58 mL)) to give R-1-(2-benzofuranyl)-2-chloroethanol (9.42 g) as a light brown oil. ¹H NMR (400 MHz, CDCl₃) δ 7.58, 7.48, 7.22-7.34, 6.79, 5.11, 3.98; HRMS (EI) found 196.0291 for C₁₀H₉ClO₂; Specific Rotation [α²⁵_{D}=−31 (c 1.03, chloroform); Chiral HPLC Analysis (Chiracel OJ): 99:1.

As described for the preparation of N-methyl R-1-(2-furyl)-2-aminoethanol, R-1-(2-benzofuranyl)-2-chloroethanol (8.45 g) is treated with methyl isocyanate (4.17 g) and Et₃N (1.74 g) in CH₂Cl₂ (50 mL) to give R-1-(2-benzofuranyl)-2-chloroethanol-N-methylcarbamate (9.75 g) as a pale brown solid. ¹H NMR (400 MHz, CDCl₃) δ 7.58, 7.49, 7.23-7.35, 6.83, 6.11, 4.84, 4.00, 2.85; IR (diffuse reflectance) 3374, 1699, 1535, 1251, 1134, 975, 924, 821, 814, 771, 748, 733, 676, 626, 613 cm⁻¹; Specific Rotation [α]²⁵_{D}=−101 (c 0.83, chloroform); Chiral HPLC Analysis (Chiracel OJ): 99:1.

As described for the preparation of N-methyl R-1-(2-furyl)-2-aminoethanol, R-1-(2-benzofuranyl)-2-chloroethanol-N-methylcarbamate (10.83 g) in THF (0.1L) is treated with KOtBu (43.1 mL, 1.0M in THF) to give 5S-3-methyl-5-(2-benzofuranyll)-2-oxazoldinone (5.25 g) as a tan solid. Mp 72-73°; ¹H NMR (400 MHz, CDCl₃) δ 7.60, 7.50, 7.35, 7.28, 6.87, 5.62, 3.87, 3.00; HRMS (ESI) found 218.0816 for C₁₂H₁₁NO₃+H₁; Specific Rotation [α]²⁵_{D}=37 (c 1.00, chloroform); Anal. Found: C, C, 66.04; H, 5.13; N, 6.38; chiral HPLC Analysis (Chiracel OJ): 96:4.

As described for the preparation of N-methyl R-1-(2-furyl)-2-aminoethanol, 5S-3-methyl-5-(2-benzofuranyll)-2-oxazoldinone (5.10 g) and 1N KOH (10 ml) in EtOH (75 ml) affords 4.20 g of (S)-1-(1-benzofuran-2-yl)-2-(methylamino)ethanol as a tan solid. Mp 89° C.; ¹H NMR (400 MHz, CDCl₃) δ 7.56, 7.47, 7.20-7.31, 6.70, 4.92, 3.10, 3.02, 2.51; HRMS (FAB) found 192.1026 for C₁₁H₁₃NO₂+H₁; Specific Rotation [α]²⁵_{D}=−30° (c 1.02, chloroform); Anal. Found: C, 68.62; H. 6.93; N, 7.25; chiral HPLC Analysis (Chiracel OJ): 96:4.

(22) Preparation of N-(4-chlorobenzyl)-8-fluoro-1-{2-[(2-hydroxyethyl)amino]-2-oxoethyl}-6-(morpholin-4-ylmethyl)-4-oxo-1,4-dihydroquinoline-3-carboxamide.

To a pressure tube containing methyl [3-{[(4-chlorobenzyl)amino]carbonyl}-8-fluoro-6-(morpholin-4-ylmethyl)-4-oxoquinolin-1(4H)-yl]acetate (4.1 g) as a suspension in THF (40 mL) is added ethanolamine (10 mL). The mixture is tightly capped, heated to 60° C. and stirred. After 2 days, the suspension is cooled to room temperature, filtered, and the precipitant washed with diethyl ether and hexanes. The residue is treated with acetonitrile (500 mL) and heated to reflux. The resulting hot suspension is quickly filtered and the filtrate cooled to room temperature and placed in a freezer. The resulting solid is collected, washed with diethyl ether, dried under a flow of air, and finally in a vacuum oven (60° C.) to yield 3.9 g of the title compound as a tan solid. Physical characteristics: ¹H NMR (d₆-DMSO) δ 2.4, 3.17, 3.4, 3.6, 4.56, 4.7, 5.21, 7.4, 7.6, 8.10, 8.35, 8.77, 10.3.

(23) Preparation of N-(4-chlorobenzyl)-1-(2-hydroxyethyl)-9-(morpholin-4-ylmethyl)-2,7-dioxo-2,3-dihydro-1H,7H-pyrido[1,2,3-de]quinoxaline-6-carboxamide.

To a flame-dried flask under a nitrogen atmosphere is added N-(4-chlorobenzyl)-8-fluoro-1-{2-[(2-hydroxyethyl)amino]-2-oxoethyl}-6-(morpholin-4-ylmethyl)-4-oxo-1,4-dihydroquinoline-3-carboxamide (3.9 g) followed by THF (60 mL). The mixture is treated with a solution of potassium tert-butoxide in THF (1 M, 18 mL). After 24 hours, the resulting dark red solution is diluted with dichloromethane and partitioned against dilute pH 4 aqueous phosphate buffer. The aqueous layer pH is adjusted to 8 and is extracted with four additional portions of dichloromethane. The combined organic layer is washed brine, dried over sodium sulfate, filtered and concentrated under reduced pressure. The resulting residue is adsorbed onto silica and flash column chromatographed on silica eluting with 4% to 15% methanol in dichloromethane. The product containing fractions are combined and concentrated under reduced pressure to yield 2.0 g of the title compound as a tan solid. Physical characteristics: ¹H NMR (d₆-DMSO) δ 2.4, 3.5-3.7, 4.12, 4.56, 4.92, 5.21, 7.4, 7.56, 7.84, 8.74, 10.4; MS (ESI+) for m/z 511 (M+H)⁺.

(24) Preparation of N-(4-chlorobenzyl)-9-(chloromethyl)-1-(2-hydroxyethyl)-2,7-dioxo-2,3-dihydro-1H,7H-pyrido[1,2,3-de]quinoxaline-6-carboxamide.

To a pressure tube containing N-(4-chlorobenzyl)-1-(2-hydroxyethyl)-9-(morpholin-4-ylmethyl)-2,7-dioxo-2,3-dihydro-1H,7H-pyrido[1,2,3-de]quinoxaline-6-carboxamide (0.26 g) is added 1,2-dicholroethane (10 mL) and ethyl chloroformate (0.5 mL). The solution is tightly capped, heated to 80° C. and stirred for 2 days. The resulting suspension is cooled to room temperature and filtered. The collected precipitant is washed with diethyl ether and dried in vacuo to yield 0.20 g of the title compound as a tan solid. Physical characteristics: ¹H NMR (d₆-DMSO) δ 3.65, 4.13, 4.57, 4.94, 5.21, 7.4, 7.69, 7.99, 8.76, 10.3; MS (ESI+) for m/z 458 (M−H).

(25) Preparation of N-(4-fluorobenzyl)-1-methyl-9-(morpholin-4-ylmethyl)-2,7-dioxo-2,3-dihydro-1H,7H-pyrido[1,2,3-de]quinoxaline-6-carboxamide

To ethyl 1-methyl-9-(morpholin-4-ylmethyl)-2,7-dioxo-2,3-dihydro-1,7H-pyrido[1,2,3-de]quinoxaline-6-carboxylate (1.00 g) (prepared using procedures analogous to those described in WO 02/04445) in a 25 mL 1-neck round bottom flask equipped with a reflux air condenser and a nitrogen inlet is added 4-fluorobenzyl amine (0.97 g) and ethylene glycol. The mixture is placed under nitrogen and is heated with stirring in an 80° C. oil bath for 24 hours. The reaction is cooled to room temperature and transferred to a separatory funnel with CH₂Cl₂ (0.25L). The mixture is washed with water (0.25 mL) and the organic phase is separated and dried over MgSO₄. The solid is removed by filtration, the filter cake is rinsed with CH₂Cl₂ (0.25L) and the combined filtrate is concentrated in vacuo to afford an orange solid. The crude material is purified on a 40S Biotage column [wet CH₂Cl₂, eluted CH₂Cl₂; CH₂Cl₂/MeOH (97.5:2.5); CH₂Cl₂/MeOH (96:4); CH₂Cl₂/MeOH (92.5:7.5); CH₂Cl₂/MeOH (90:10)]. The product fractions are combined to provided 0.678 g of N-(4-fluorobenzyl)-1-methyl-9-(morpholin-4-ylmethyl)-2,7-dioxo-2,3-dihydro-1H,7H-pyrido[1,2,3-de]quinoxaline-6-carboxamide as an ivory solid. Physical charteristics: mp 222-224° C.; ¹H-NMR (400 MHz, DMSO-d₆): δ 10.25, 8.73, 7.85, 7.39, 7.17, 5.21, 4.55, 3.61, 3.59; 2.40; HRMS (ESI) found 465.1938 for C₂₅H₂₅FN₄O₄+H₁; Anal. Found: C, 64.27; H, 5.57; N, 11.94; F, 4.08.

(26) Preparation of N-(4-fluorobenzyl)-1-methyl-9-(chloromethyl)-2,7-dioxo-2,3-dihydro-1H,7H-pyrido[1,2,3-de]quinoxaline-6-carboxamide

To a suspension of N-(4-fluorobenzyl)-1-methyl-9-(morpholin-4-ylmethyl)-2,7-dioxo-2,3-dihydro-1H,7H-pyrido[1,2,3-de]quinoxaline-6-carboxamide (1.35 g) in CHCl₃ (25 mL) in a 50 mL 1-neck round bottom flask equipped with a reflux condenser and a nitrogen inlet is added ClCO₂Et (0.79 g) and i-Pr₂NEt (0.94 g). The mixture is warmed under reflux for 2 hours, then is cooled to room temperature and diluted with Et₂O (0.1L). The light tan solid is isolated by suction filtration, rinsed with Et₂O (0.1L), and dried in vacuo to give N-(4-fluorobenzyl)-1-methyl-9-(chloromethyl)-2,7-dioxo-2,3-dihydro-1H,7H-pyrido[1,2,3-de]quinoxaline-6-carboxamide (0.98 g). Physical characteristics: ¹H-NMR (400 MHz, DMSO-d₆): δ 10.25, 8.75, 7.98, 7.55, 7.38, 7.16, 5.21, 4.94, 4.55, 3.93.

Preparation 27

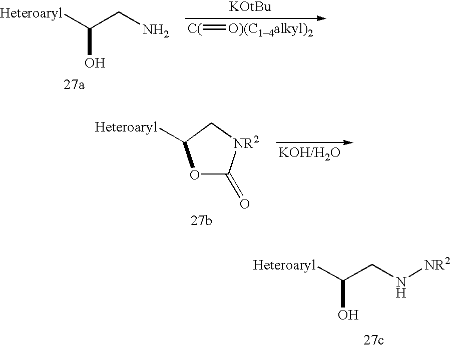

As shown in the preparation 27 above, the hetarylamino alcohol starting material, 27a, is converted to the oxazolidinone 27b by treatment with dimethyl carbonate (or a C₁₋₄alkyl carbonate) and a base such as potassium tert-butoxide in DMF. The oxazolidinone, wherein R² is C₁₋₄alkyl, can have the optical purity enhanced by crystallization or chiral chromatography. Basic aqueous hydrolysis of the oxazolidinone with a base such as potassium hydroxide affords the optically enhanced N-alkylated heteroarylamino alcohol 27c, wherein heteroaryl is furan and those hetroaryls as defined in the detailed description of the invention.

Step 1: Preparation of 5R-3-Methyl-5-(2-furyl)-2-oxazolidinone.

A 250 mL round-bottomed flask equipped with an overhead stirrer, reflux condenser, thermocouple and an addition funnel is charged with (R)-2-amino-1-(2-furyl)ethanol (10 g) and potassium t-butoxide (10.6 g). Anhydrous DMF is charged at such a rate as to keep the temp less than 50° C. The reaction is heated to 80° C. (internal temp), the addition funnel is charged with dimethyl carbonate (50 mL), and the liquid is added in 0.2 equiv portions at such a rate as to control the evolution of gas. Once addition of dimethyl carbonate is complete, the temperature is raised to reflux (about 100° C.), and maintained for approximately 12 h. The reaction mixture is cooled to less than 60° C., poured into water (100 mL) and extracted with isopropyl acetate (100 mL). The layers are separated, and the water layer is extracted with additional isopropyl acetate (2×100 mL). The combined organic layers are washed with water (100 ML) and dried over sodium sulfate and magnesol for 10 min. The solids are removed via vacuum filtration, and the organic layers are concentrated in vacuo. The resulting oil is crystallized from MTBE (2 mL/g) to provide 10.25 g of (5R)-5-(2-furyl)-3-methyl-1,3-oxazolidin-2-one. Physical characteristics. ¹H NMR (CDCl₃, 400 MHz) δ 7.46, 6.49, 6.39, 5.47, 3.78, 2.97.

Crystallization of 5R-3-Methyl-5-(2-furyl)-2-oxazolidinone.

A 250 mL 3-neck rb flask equipped with an overhead stirrer, and thermocouple, is charged with 58.3 g (350 mmol, 98.4% ee) of 5R-3-Methyl-5-(2-furyl)-2-oxazolidinone and 116.6 mL of MTBE. The resulting suspension is heated to 50° C. (internal), resulting in the formation of a solution. The solution is allowed to cool to 26° C. over 1.5 h, at which point a white slurry is obtained. The slurry is cooled to 5° C. and aged for 1.5 h. The solids are collected by vacuum filtration, rinsing with 5° C. MTBE (1 mL/g), to give an 85% recovery (49.5 g) of 5R-3-Methyl-5-(2-furyl)-2-oxazolidinone of enriched optical and chemical purity. Physical Characteristics: ¹H NMR (400 MHz, CDCl₃) δ 7.46, 6.49, 6.39, 5.47, 3.78, 2.97. ¹³C NMR (100 MHz, CDCl₃, DEPT) δ 157.5, 149.8, 143.7, 110.6, 109.9, 67.5, 50.4, 31.0. Melt Solvate: 0.04% MTBE; KF: 0.00%; Specific Rotation [α^{]D}₂₅=−113° (c=1.00, CH₂Cl₂); Chiral HPLC Analysis: 0.06:99.94; 99.9% ee.

Step 2: Preparation of N-Methyl R-1-(2-furyl)-2-aminoethanol [used in examples 11,23, 31].

A round-bottomed flask equipped with an overhead stirrer, reflux condenser and nitrogen inlet is charged with (5R)-5-(2-furyl)-3-methyl-1,3-oxazolidin-2-one (47.1 g). A 1 M solution of KOH (987 mL) is added and the resulting solution is heated at 50° C. When complete, the flask is charged with NaCl (310 g) and MTBE (470 mL). The aqueous layer is separated and further extracted twice with a solution of MTBE (470 mL) and CH₂Cl₂ (23 mL). The combined organic layers are dried (MgSO₄), filtered, and concentrated to afford 38.0 g of the title compound. Physical characteristics. ¹H NMR (DMSO, 400 MHz) δ 7.52, 6.36, 6.24, 4.60, 2.71, 2.28; ¹³C NMR (DMSO, 100 MHz) δ 157.0, 141.6, 110.1, 105.6, 65.2, 56.2, 35.9; [α]²²_{D}=+34° (EtOH, c=1.0).

Preparation 28

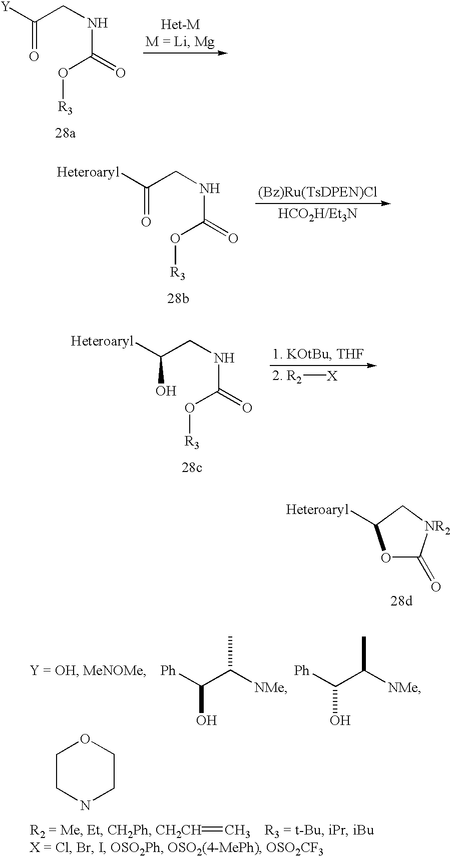

As shown in the preparation 28 above, 28a is converted to 28b by treatment with organometallic in THF. The ketone of 28b is reduced to the chiral alcohol 28c using a chiral Ruthenium catalyst and formic acid triethylamine complex. Cyclization under basic conditions and alkylated to give 28d, wherein hetaryl is furan and those hetaryls defined in the detailed description of the invention.

Step 1: Preparation of tert-Butyl (2-oxo-2-(2-furyl)-ethyl)carbamate

To a solution of furan (3.0 g) in anhydrous THF (30 mL) at 0° C. is added nBuLi (2.5 M in hexane) at such a rate as to keep the internal temperature less than 25° C. After stirring at 0-5° C. for 50 min, the reaction mixture is cooled to between −30 and −20° C., and a suspension of N-(tert-butoxycarbonyl)glycine N′-methoxy-N′-methylamide [4.36 g)] in THF (15 mL) is added drop-wise by large-bore cannula. The resulting slurry is allowed to warm to 5° C. over a 6 h period, at which time GC indicated less than 4% N-(tert-butoxycarbonyl)glycine N′-methoxy-N′-methylamide remained. The reaction is quenched by the addition of aqueous citric acid (20 mL; pH=5.0 after quench) and diluted with CH₂Cl₂ (40 mL). The resulting phases are separated, and the aqueous is back extracted twice with CH₂Cl₂ (20 mL). The organic layers are combined, dried over sodium sulfate, and concentrated in vacuo (50 torr) to provide 4.1 g (18.2 mmol, 91%; 91% potency by GC) of tert-Butyl (2-oxo-2-(2-furyl)-ethyl)carbamate 3 as a light yellow solid. Physical characteristics: ¹H NMR (CDCl₃, 400 MHz), δ =7.60, 7.28, 6.57, 5.39, 4.50, 1.47 ppm. ¹³C NMR (100 MHz, CDCl₃, DEPT) δ 183.9, 155.7, 150.8,79.9, 146.8, 117.7, 112.4,46.9,28.3.

Step 2: Preparation 4 of (R)-1-(2-furyl)-2-((tert-butoxycarbonyl)amino)ethanol ((η⁶ C₆H₆)Ru[(R,R)-TsDPEN]Cl): A 50 mL flask is charged with 50 mg of [(η⁶C₆H₆)RuCl₂]₂, 75 mg (R)(R)-TsDPEN, 2.5 mL i-PrOH (anhydrous from Aldrich) and 80 uL NEt₃ (F.W.=101, 0.57 mmol), a reflux condenser attached and, under nitrogen, the reaction mixture is heated to 75° C. for 1 hr. The reaction is then cooled to 0° C. giving a dark orange solid which is collected by filtration. The solid is washed with diethyl ether and air-dried giving 71 mg of product (F.W.=578.9, 0.123 mmol, 61% as an air stable material).

To a 50 mL RB flask in the glovebox is added ca. 4 mg of (η⁶C₆H₆)Ru[(R,R)-TsDPEN]Cl (prepared above) followed by 5 mL of a NEt₃/HCO₂H (1:1.5 molar ratio). This is allowed to stir at r.t. for 20 minutes giving a pale yellow solution. To this solution is added 1 g of tert-butyl (2-oxo-2-(2-furyl)-ethyl)carbamate and the reaction mixture is stirred at r.t. for 22 hr (monitored by HPLC). By HPLC analysis a 100% conversion of ketone to >99% (by area) alcohol is achieved. The crude reaction is poured into 75 mL H₂O. This is extracted 2×75 mL EtOAc. The combined organic layers are extracted with 1×50 mL 1 M aq. NaHCO₃ and then with 1×50 mL brine. The organic layer is dried over MgSO₄, filtered and concentrated to give (R)-1-(2-furyl)-2-((tert-butoxycarbonyl)amino)ethanol as a yellow oil, Physical Characteristics: LCMS m/z=250, 154; Uv-vis λ=216. ¹H NMR (CD₃CN) δ 7.40, 6.33, 6.25, 5.39, 4.61, 3.62, 3.30 1.36; Chiral HPLC analysis: 3:97; 94%ee.

Step 3: preparation of 5R-3-Methyl-5-(2-furyl)-2-oxazoldinone

A round-bottomed flask equipped with an overhead stirrer, reflux condensor, thermocouple and an addition funnel is charged with (R)-1-(2-furyl)-2-((tert-butoxycarbonyl)amino)ethanol (200 g) and anhydrous tetrahydrofuran (2.0 L). A 1.0 M solution of potassium tert-butoxide (1.06 L) is charged at such a rate as to keep the temp less than 35° C. Once all of the starting material has been consumed, as determined by HPLC, methyl iodide (63.5 mL) is added drop-wise to the suspension. The suspension is then allowed to stir at 25-35 degrees until all of the intermediate normethyl oxazolidinone is consumed (HPLC). The reaction is quenched with water (2.0 L) and diluted with isopropyl acetate (2.0 L). The biphasic mixture is agitated, the phases are separated, and the aqueous is back extracted with isopropyl acetate (2×2.0 L). The organic layers are combined, dried over sodium sulfate (500 g) and concentrated to give 147 g of 5R-3-Methyl-5-(2-furyl)-2-oxazoldinone 28D. Physical characteristics. ¹H NMR (CDCl₃, 400 MHz) δ 7.46, 6.49, 6.39, 5.47, 3.78, 2.97.

Preparation 29

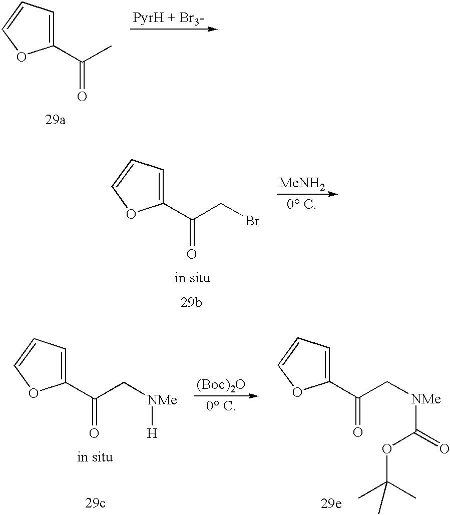

Preparation of tert-Butyl (2-oxo-2-(2-furyl)-ethyl)carbamate

A 250 mL RB flask is charged, in order, with; 10.56 g 2-acetylfuran, 100 mL CH₂Cl₂ and 30.3 g pyridinium tribromide. The reaction is allowed to stir at RT for 30 min After 30 min the reaction mass is cooled to 0° C. and nitrogen is purged over the surface of the reaction mixture for 10 min to remove HBr. To the reaction mixture, cooled to 0° C., is added 200 mL of 2 M MeNH₂ (in THF) all at once. This reaction mixture is allowed to stir at 0° C. for 10 min after which time nitrogen is purged through the reaction mixture for 15 min to remove excess MeNH₂. At 0° C., 42 g of (Boc)₂—O in 50 mL THF is added all at once to the reaction mixture generated above. The reaction is allowed to warm to RT and after 45 min an additional 5 g (Boc)₂—O is added and the reaction mixture is stirred for an additional 30 min. The resultant reaction mixture is concentrated in vacuo. The residue is extracted with diethyl ether/H₂O. The ether layer is extracted with a second portion of H₂O and then with brine. The combined aqueous layers are extracted with diethyl ether. The combined ether layers are dried over MgSO₄, filtered and concentrated to a purple colored oil. The residue is dissolved in EtOAc, filtered through a plug of silica gel using 50:50 ethyl acetate:hexane to wash the silica gel. The filtrate is concentrated giving an oil. The oil is then chromatographed on silica gel using ethyl acetae/hexane (1:4) giving 7.81 g of a white solid. Physical Characteristics: LCMS m/z=262, 162, 140; Uv-vis λₘₐₓ=200, 226, 268. ¹H NMR (DMSO-d₆) δ 7.98, 7.46, 6.69, 4.44 2.81, 2.78, 1.35, 1.21 ppm; ¹³C NMR(CDCl₃) δ 184.7, 184.6; 156.3; 155.8; 146.7, 117.5; 117.3; 112.5; 113.4; 80.2; 55.4; 54.7; 35.9; 28.5; 28.3 ppm.

Preparation 30

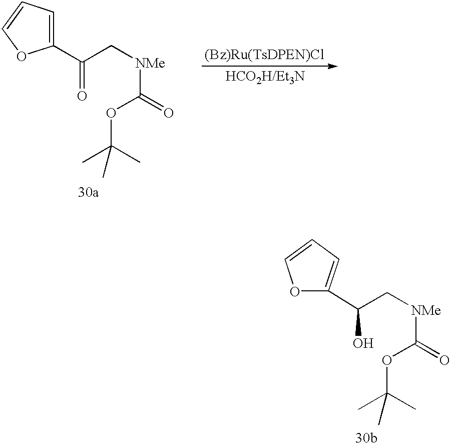

In the glovebox a fresh solution of formic acid and triethylamine is made by the addition of 13.2 g NEt₃ (F.W.=101, 0.13 mol, Aldrich) to 9 g 98% formic acid (F.W.=46, 0.196 mol, Aldrich), exothermic reaction.

To a glass vial is added 5 mg of (η⁶C₆H₆)Ru[(R,R)-TsDPEN]Cl followed by 7 mL of the NEt₃/HCO₂H. This is allowed to stir at r.t. for 20 minutes giving a pale yellow solution. To this solution is added 1 g of the o-boc-methylaminoketone substrate (F.W.=239, 4.18 mmol) and the reaction mixture is stirred at r.t. for 3 d (monitored by HPLC). By HPLC analysis a 100% conversion of ketone to >95% (by area) alcohol is achieved.

The product is recovered by pouring the crude reaction into a mixture of 150 mL ethyl acetate and 150 mL H₂O. The aqueous layer is extracted with a second 150 mL portion of ethyl acetate. The combined organic layers are extracted with aq. NaHCO₃ (75 mL) and then brine (75 mL). The organic layer ws dried over MgSO₄, filtered and concentrated giving 1.03 g (102%) of alcohol (PHA-774326) which is analyzed by chiral HPLC giving 98.1% ee of (+) isomer. ¹H NMR (CD₃CN) δ 7.41 (s, 1H), 6.34 (br s, 1H), 6.25 (d, J=2.4 Hz, 1H), 4.76 (br m, 1H), 3.46 (br overlapping, 3H), 2.74 (s, 3H), 1.37 (s, 9H).

What is claimed is:

1. A compound of formula I 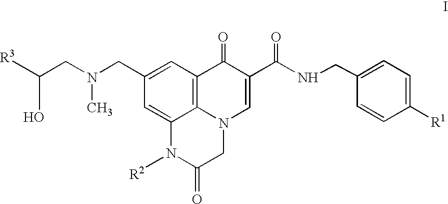
or a pharmaceutically acceptable salt thereof
wherein R′ is F or Cl;
R² is C₁₋₄alkyl, optionally substituted by OH or OC₁₋₄alkyl;
R³ is aryl or heteroaryl, optionally substituted by one to three C₁₋₂alkyl, OH, OC₁₋₂alkyl or CN;
aryl is a phenyl or benzyl radical optionally fused to a benzene ring; and
heteroaryl is a 5- or 6-membered aromatic ring having at least one heteroatom selected from the group consisting of O, S and N(X) wherein X is absent or H, wherein heteroaryl is optionally fused to a benzene ring.

2. A compound of claim 1 wherein R¹ is Cl.

3. A compound of claim 1 wherein R¹ is F.

4. A compound of claim 1 wherein R² is methyl.

5. A compound of claim 1 wherein R² is ethyl optionally substituted with OH.

6. A compound of claim 1, 2, 3, 4 or 5 wherin R³ is phenyl.

7. A compound of claim 1, 2, 3, 4 or 5 wherin R³ is phenyl substituted by one or two OH or OCH₃.

8. A compound of claim 1, 2, 3, 4, or 5 wherin R³ is a 5-membered aromatic ring having at least one heteroatom selected from the group consisting of O, S and N(X) wherein X is absent or H.

9. A compound of claim 8 wherein R³ is furyl, thien or thiazol, wherein R³ is optionally fused to a benzene ring.

10. A compound of claim 9 wherein R³ is optionally substituted by one to two methyl, OH, OCH₃ or CN.

11. A compound of claim 8 wherein R³ is 1-benzofuran-2-yl, 3-furyl, 2-furyl, 3-furyl, 5-methyl-2-furyl, 2,5-dimethyl-3-furyl, 2-thien, 1-benzothien-3-yl, 5-cyanothien-2-yl, or 1,3-thiazol-2yl.

12. A compound of claim 1, 2, 3, 4 or 5 wherin R³ is a 6-membered aromatic ring having at least one heteroatom selected from the group consisting of O, S and N.

13. A compound of claim 12 wherein R³ is pyridinyl, pyrimidinyl, or pyrazinyl, wherein R³ is optionally fused to a benzene ring.

14. A compound of claim 13 wherein R³ is optionally substituted by one to two methyl, OH, OCH₃ or CN.

15. A compound of claim 12 wherein R³ is pyridin-2-yl, 6-methylpyridin-2-yl, pyridin-3-yl, quinolin-2-yl, or pyrimidin-2-yl.

16. The formula I of claim 1 which is formula IA:

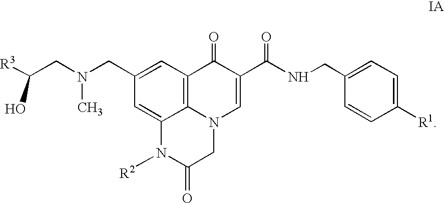

17. A compound of claim 16 wherein R¹ is Cl.

18. A compound of claim 16 wherein R¹ is F.

19. A compound of claim 16 wherein R² is methyl.

20. A compound of claim 16 wherein R² is ethyl optionally substituted with OH.

21. A compound of claim 16, 17, 18, 19 or 20 wherin R³ is phenyl.

22. A compound of claim 16, 17, 18, 19 or 20 wherin R³ is phenyl substituted by one or two OH or OCH₃.

23. A compound of claim 16, 17, 18, 19 or 20 wherin R³ is a 5-membered aromatic ring having at least one heteroatom selected from the group consisting of O, S and N(X) wherein X is absent or H.

24. A compound of claim 23 wherein R³ is furyl, thien or thiazol, wherein R³ is optionally fused to a benzene ring.

25. A compound of claim 24 wherein R³ is optionally substituted by one to two methyl, OH, OCH₃ or CN.

26. A compound of claim 23 wherein R³ is 1-benzofuran-2-yl, 3-furyl, 2-furyl, 3-furyl, 5-methyl-2-furyl, 2,5-dimethyl-3-furyl, 2-thien, 1-benzothien-3-yl, 5-cyanothien-2-yl, or 1,3-thiazol-2yl.

27. A compound of claim 16, 17, 18, 19 or 20 wherin R³ is a 6-membered aromatic ring having at least one heteroatom selected from the group consisting of O, S and N.

28. A compound of claim 27 wherein R³ is pyridinyl, pyrimidinyl, or pyrazinyl, wherein wherein R³ is optionally fused to a benzene ring.

29. A compound of claim 28 wherein R³ is optionally substituted by one to two methyl, OH, OCH₃ or CN.

30. A compound of claim 27 wherein R³ is pyridin-2-yl, 6-methylpyridin-2-yl, pyridin-3-yl, quinolin-2-yl, or pyrimidin-2-yl.

31. A compound of claim 1 which is
(1) N-(4-chlorobenzyl)-9-{[(2-hydroxy-2-phenylethyl) (methyl)amino]methyl}-1-methyl-2,7-dioxo-2,3-dihydro-1H,7H-pyrido[1,2,3-de]quinoxaline-6-carboxamide,
(2) rac N-(4-chlorobenzyl)-9-{[[2-hydroxy-2-(4-hydroxyphenyl)ethyl](methyl)amino]methyl}-1-methyl-2,7-dioxo-2,3-dihydro-1H,7H-pyrido[1,2,3-de]quinoxaline-6-carboxamide,
(3) rac 9-{[[2-(1-benzofuran-2-yl)-2-hydroxyethyl](methyl)amino]methyl}-N-(4-chlorobenzyl)-1-methyl-2,7-dioxo-2,3-dihydro-1H,7H-pyrido[1,2,3-de]quinoxaline-6-carboxamide,
(4) rac N-(4-chlorobenzyl)-9-{[[2-hydroxy-2-(5-methyl-2-furyl)ethyl](methyl)amino]methyl}-1-methyl-2,7-dioxo-2,3-dihydro-1H,7H-pyrido[1,2,3-de]quinoxaline-6-carboxamide,
(5) N-(4-chlorobenzyl)-9-{[[(2R)-2-(2-furyl)-2-hydroxyethyl](methyl)amino]methyl}-1-methyl-2,7-dioxo-2,3-dihydro-1H,7H-pyrido[1,2,3-de]quinoxaline-6-carboxamide,
(6) N-(4-chlorobenzyl)-9-{[[(2S)-2-hydroxy-2-phenylethyl](methyl)amino]methyl}-1-methyl-2,7-dioxo-2,3-dihydro-1H,7H-pyrido[1,2,3-de]quinoxaline-6-carboxamide,
(7) N-(4-chlorobenzyl)-9-{[[(2S)-2-hydroxy-2-pyridin-3-ylethyl](methyl)amino]methyl}-1-methyl-2,7-dioxo-2,3-dihydro-1H,7H-pyrido[1,2,3-de]quinoxaline-6-carboxamide,
(8) rac 9-{[[2-(1-benzothien-3-yl)-2-hydroxyethyl](methyl)amino]methyl}-N-(4-chlorobenzyl)-1-methyl-2,7-dioxo-2,3-dihydro-1H,7H-pyrido[1,2,3-de]quinoxaline-6-carboxamide,
(9) rac N-(4-chlorobenzyl)-9-{[(2-hydroxy-2-quinolin-2-ylethyl) (methyl)amino]methyl}-1-methyl-2,7-dioxo-2,3-dihydro-1H,7H-pyrido[1,2,3-de]quinoxaline-6-carboxamide,
(10) N-(4-chlorobenzyl)-9-{[[(2R)-2-hydroxy-2-pyrazin-2-ylethyl](methyl)amino]methyl}-1-methyl-2,7-dioxo-2,3-dihydro-1H,7H-pyrido[1,2,3-de]quinoxaline-6-carboxamide, or
(11) rac 9-{[[2-(1-benzofuran-2-yl)-2-hydroxyethyl](methyl)amino]methyl}-N-(4-fluorobenzyl)-1-methyl-2,7-dioxo-2,3-dihydro-1H,7H-pyrido[1,2,3-de]quinoxaline-6-carboxamide.
(12) 9-{[[(2R)-2-(1-benzofuran-2-yl)-2-hydroxyethyl](methyl)amino]methyl}-N-(4-chlorobenzyl)-1-methyl-2,7-dioxo-2,3-dihydro-1H,7H-pyrido[1,2,3-de]quinoxaline-6-carboxamide, or
(13) 9-{[[(2R)-2-(1-benzofuran-2-yl)-2-hydroxyethyl](methyl)amino]methyl}-N-(4-fluorobenzyl)-1-methyl-2,7-dioxo-2,3-dihydro-1H,7H-pyrido[1,2,3-de]quinoxaline-6-carboxamide.

32. A compound of claim 1 which is
(1) rac N-(4-chlorobenzyl)-9-{[(2-hydroxy-2-phenylethyl)(methyl)amino]methyl}-1-methyl-2,7-dioxo-2,3-dihydro-1H,7H-pyrido[1,2,3-de]quinoxaline-6-carboxamide,
(2) rac N-(4-chlorobenzyl)-9-{[[2-(3-furyl)-2-hydroxyethyl](methyl)amino]methyl}-1-methyl-2,7-dioxo-2,3-dihydro-1H,7H-pyrido[1,2,3-de]quinoxaline-6-carboxamide,
(3) rac N-(4-chlorobenzyl)-9-{[[2-(2-furyl)-2-hydroxyethyl](methyl) amino]methyl}-1-methyl-2,7-dioxo-2,3-dihydro-1H,7H-pyrido[1,2,3-de]quinoxaline-6-carboxamide,
(4) rac N-(4-chlorobenzyl)-9-{[[2-(2,5-dimethyl-3-furyl)-2-hydroxyethyl](methyl)amino]methyl}-1-methyl-2,7-dioxo-2,3-dihydro-1H,7H-pyrido[1,2,3-de]quinoxaline-6-carboxamide,
(5) rac N-(4-chlorobenzyl)-9-{[[2-hydroxy-2-(6-methylpyridin-2-yl)ethyl](methyl)amino]methyl}-1-methyl-2,7-dioxo-2,3-dihydro-1H,7H-pyrido[1,2,3-de]quinoxaline-6-carboxamide,
(6) N-(4-chlorobenzyl)-9-{([[(2R)-2-hydroxy-2-pyridin-2-ylethyl](methyl)amino]methyl}-1-methyl-2,7-dioxo-2,3-dihydro-1H,7H-pyrido[1,2,3-de]quinoxaline-6-carboxamide,
(7) rac N-(4-chlorobenzyl)-9-{[[2-hydroxy-2-(4-hydroxy-3-methoxyphenyl)ethyl](methyl)amino]methyl}-1-methyl-2,7-dioxo-2,3-dihydro-1H,7H-pyrido[1,2,3-de]quinoxaline-6-carboxamide,
(8) N-(4-chlorobenzyl)-9-{[[(2S)-2-hydroxy-2-thien-3-ylethyl](methyl)amino]methyl}-1-methyl-2,7-dioxo-2,3-dihydro-1H,7H-pyrido [1,2,3-de]quinoxaline-6-carboxamide,
(9) rac N-(4-chlorobenzyl)-9-{[(2-hydroxy-3-phenylpropyl)(methyl)amino]methyl}-1-methyl-2,7-dioxo-2,3-dihydro-1H,7H-pyrido[1,2,3-de]quinoxaline-6-carboxamide,
(10) rac N-(4-chlorobenzyl)-9-{[[2-hydroxy-2-(3-methoxyphenyl)ethyl](methyl)amino]methyl}-1-methyl-2,7-dioxo-2,3-dihydro-1H,7H-pyrido[1,2,3-de]quinoxaline-6-carboxamide,
(11) rac N-(4-chlorobenzyl)-9-[(2-hydroxy-2-pyrimidin-2-ylethyl)(methyl)amino]methyl}-1-methyl-2,7-dioxo-2,3-dihydro-1H,7H-pyrido[1,2,3-de]quinoxaline-6-carboxamide,
(12) rac N-(4-chlorobenzyl)-9-{[[2-(5-cyanothien-2-yl)-2-hydroxyethyl](methyl)amino]methyl}-1-methyl-2,7-dioxo-2,3-dihydro-1H,7H-pyrido[1,2,3-de]quinoxaline-6-carboxamide,
(13) N-(4-chlorobenzyl)-9-1{[[(2R)-2-hydroxy-2-(1,3-thiazol-2-yl)ethyl](methyl)amino]methyl}-1-methyl-2,7-dioxo-2,3-dihydro-1H,7H-pyrido[1,2,3-de]quinoxaline-6-carboxamide,
(14) N-(4-fluorobenzyl)-9-{[[(2R)-2-(2-furyl)-2-hydroxyethyl](methyl)amino]methyl}-1-methyl-2,7-dioxo-2,3-dihydro-1H,7H-pyrido[1,2,3-de]quinoxaline-6-carboxamide,
(15) N-(4-fluorobenzyl)-9-{[[(2R)-2-hydroxy-2-pyridin-2-ylethyl](methyl)amino]methyl}-1-methyl-2,7-dioxo-2,3-dihydro-1H,7H-pyrido[1,2,3-de]quinoxaline-6-carboxamide,
(16) N-(4-fluorobenzyl)-9-{[[(2S)-2-hydroxy-2-pyridin-3-ylethyl](methyl)amino]methyl}-1-methyl-2,7-dioxo-2,3-dihydro-1H,7H-pyrido[1,2,3-de]quinoxaline-6-carboxamide,
(17) rac 9-{[[2-(1-benzofuran-2-yl)-2-hydroxyethyl](methyl)amino]methyl}-N-(4-chlorobenzyl)-1-(2-hydroxyethyl)-2,7-dioxo-2,3-dihydro-1H,7H-pyrido[1,2,3-de]quinoxaline-6-carboxamide,
(18) N-(4-fluorobenzyl)-9-{[[(2R)-2-hydroxy-2-(1,3-thiazol-2-yl)ethyl](methyl)amino]methyl}-1-methyl-2,7-dioxo-2,3-dihydro-1H,7H-pyrido [1,2,3-de]quinoxaline-6-carboxamide,
(19) N-(4-fluorobenzyl)-9-{[[(2S)-2-hydroxy-2-thien-3-ylethyl](methyl)amino]methyl}-1-methyl-2,7-dioxo-2,3-dihydro-1H,7H-pyrido[1,2,3-de]quinoxaline-6-carboxamide,
(20) N-(4-fluorobenzyl)-9-{[[(2S)-2-hydroxy-2-phenylethyl](methyl)amino]methyl}-1-methyl-2,7-dioxo-2,3-dihydro-1H,7H-pyrido[1,2,3-de]quinoxaline-6-carboxamide,
(21) N-(4-fluorobenzyl)-9-{[[(2R)-2-hydroxy-2-pyrazin-2-ylethyl](methyl)amino]methyl}-1-methyl-2,7-dioxo-2,3-dihydro-1H,7H-pyrido[1,2,3-de]quinoxaline-6-carboxamide,
(22) N-(4-chlorobenzyl)-9-{[[(2R)-2-(2-furyl)-2-hydroxyethyl](methyl)amino]methyl}-1-(2-hydroxyethyl)-2,7-dioxo-2,3-dihydro-1H,7H-pyrido[1,2,3-de]quinoxaline-6-carboxamide,
(23) N-(4-fluorobenzyl)-9-{[[(2R)-2-hydroxy-2-thien-2-ylethyl](methyl)amino]methyl}-1-methyl-2,7-dioxo-2,3-dihydro-1H,7H-pyrido[1,2,3-de]quinoxaline-6-carboxamide,
(24) 9-{[[(2S)-2-(1-benzofuran-2-yl)-2-hydroxyethyl](methyl)amino]methyl}-N-(4-chlorobenzyl)-1-methyl-2,7-dioxo-2,3-dihydro-1H,7H-pyrido[1,2,3-de]quinoxaline-6-carboxamide, or
(25) 9-{[[(2S)-2-(1-benzofuran-2-yl)-2-hydroxyethyl](methyl)amino]methyl}-N-(4-fluorobenzyl)-1-methyl-2,7-dioxo-2,3-dihydro-1H,7H-pyrido[1,2,3-de]quinoxaline-6-carboxamide.

33. A compound of claim 1 which is
(1) N-(4-chlorobenzyl)-9-{[[(2R)-2-hydroxy-2-(4-hydroxyphenyl)ethyl](methyl)amino]methyl}-1-methyl-2,7-dioxo-2,3-dihydro-1H,7H-pyrido[1,2,3-de]quinoxaline-6-carboxamide,
(2) N-(4-chlorobenzyl)-9-{[[(2S)-2-hydroxy-2-(4-hydroxyphenyl)ethyl](methyl)amino]methyl}-1-methyl-2,7-dioxo-2,3-dihydro-1H,7H-pyrido[1,2,3-de]quinoxaline-6-carboxamide,
(3) N-(4-chlorobenzyl)-9-{[[(2R)-2-hydroxy-2-(5-methyl-2-furyl)ethyl](methyl)amino]methyl}-1-methyl-2,7-dioxo-2,3-dihydro-1H,7H-pyrido[1,2,3-de]quinoxaline-6-carboxamide,
(4) N-(4-chlorobenzyl)-9-{[[(2S)-2-hydroxy-2-(5-methyl-2-furyl)ethyl](methyl)amino]methyl}-1-methyl-2,7-dioxo-2,3-dihydro-1H,7H-pyrido[1,2,3-de]quinoxaline-6-carboxamide,
(5) 9-{[[(2R)-2-(1-benzothien-3-yl)-2-hydroxyethyl](methyl)amino]methyl}-N-(4-chlorobenzyl)-1-methyl-2,7-dioxo-2,3-dihydro-1H,7H-pyrido[1,2,3-de]quinoxaline-6-carboxamide,
(6) 9-{[[(2S)-2-(1-benzothien-3-yl)-2-hydroxyethyl](methyl)amino]methyl}-N-(4-chlorobenzyl)-1-methyl-2,7-dioxo-2,3-dihydro-1H,7H-pyrido[1,2,3-de]quinoxaline-6-carboxamide,
(7) N-(4-chlorobenzyl)-9-{[((2R)-2-hydroxy-2-quinolin-2-ylethyl)(methyl)amino]methyl}-1-methyl-2,7-dioxo-2,3-dihydro-1H,7H-pyrido[1,2,3-de]quinoxaline-6-carboxamide,
(8) N-(4-chlorobenzyl)-9-{[((2S)-2-hydroxy-2-quinolin-2-ylethyl)(methyl)amino]methyl}-1-methyl-2,7-dioxo-2,3-dihydro-1H,7H-pyrido[1,2,3-de]quinoxaline-6-carboxamide
(9) 9-{[[(2R)-2-(1-benzofuran-2-yl)-2-hydroxyethyl](methyl)amino]methyl}-N-(4-fluorobenzyl)-1-methyl-2,7-dioxo-2,3-dihydro-1H,7H-pyrido[1,2,3-de]quinoxaline-6-carboxamide, or
(10) 9-{[[(2S)-2-(1-benzofuran-2-yl)-2-hydroxyethyl](methyl)amino]methyl}-N-(4-fluorobenzyl)-1-methyl-2,7-dioxo-2,3-dihydro-1H,7H-pyrido[1,2,3-de]quinoxaline-6-carboxamide.

34. A pharmaceutical composition comprising a compound of claim 1 and a pharmaceutically acceptable carrier.

35. A method for the treatment of herpesviruses infections comprising administering to a mammal in need thereof a compound of claim 1 or a pharmaceutically acceptable salt thereof.

36. The method of claim 35 wherein the mammal is a human.

37. The method of claim 35 wherein the mammal is an animal.

38. The method of claim 35 wherein said herpesviruses is herpes simplex virus types 1, herpes simplex virus types 2, varicella zoster virus, cytomegalovirus, Epstein-Barr virus, human herpes viruses 6, human herpes viruses 7 or human herpes viruses 8.

39. The method of claim 35 wherein said herpesviruses is human cytomegalovirus.

40. The method of claim 35 wherein said herpesviruses is varicella zoster virus or Epstein-Barr virus.

41. The method of claim 35 wherein said herpesviruses is herpes simplex virus types 1 or herpes simplex virus types 2.

42. A method for the treatment of atherosclerosis and restenosis comprising administering to a mammal in need of such treatment, a compound of formula I or a pharmaceutically acceptable salt thereof.

43. The method of claim 35 or 42 wherein the compound of claim 1 is administered orally, parenterally, topically, rectally, nasally, sublingually or transdermally.

44. The method of claim 35 or 42 wherein the compound of claim 1 is administered orally, parenterally or topically.

45. The method of claim 35 or 42 wherein the compound of claim 1 is in an amount of from about 0.1 to about 300 mg/kg of body weight.

46. The method of claim 35 or 42 wherein the compound of claim 1 is in an amount of from about 1 to about 30 mg/kg of body weight.

47. A method for inhibiting a viral DNA polymerase, comprising contacting the polymerase with an effective inhibitory amount of a compound of claim 1.

48. A method for the treatment of herpesviruses infections comprising administering to a mammal in need of such treatment an effective amount of a compound of claim 1 or a pharmaceutically acceptable salt thereof, and one or more other antiviral agents.

49. A method for the treatment of atherosclerosis and restenosis comprising administering to a mammal in need of such treatment, a compound of formula I or a pharmaceutically acceptable salt thereof, and one or more other antiviral agents.

50. The method of claim 48 or 49 wherein the antiviral agent is selected from the group consisting of Acyclovir, Penciclovir, Famciclovir, Valaciclovir, Ganciclovir, Valganciclovir, Foscarnet, and Cidofovir.

51. The method of claim 48 wherein the compound of formula I and the antiviral agents are administered concurrently.

52. The method of claim 48 wherein the compound of formula I and the antiviral agents are administered concomitantly.

53. An intermediate useful for the preparation of a compound of formula I which is:
(1) 1-(1-benzothien-3-yl)-2-(methylamino)ethanol,
(2) 1-(2,5-dimethyl-3-furyl)-2-(methylamino)ethanol,
(3) 2-(methylamino)-1-(quinolin-2-yl)ethanol,
(4) 2-(methylamino)-1-(5-methyl-2-furyl)ethanol,
(5) 1-(3-furyl)-2-(methylamino)ethanol,
(6) 2-(methylamino)-1-(6-methylpyridin-2-yl)ethanol,
(7) 5-[1-hydroxy-2-(methylamino)ethyl]thiophene-2-carbonitrile,
(8) 2-(methylamino)-1-pyrimidin-2-ylethanol,
(9) R-2-(1-hydroxy-2-N-methylamino-ethyl)-pyridine,
(10) N-methyl R-1-(2-furyl)-2-aminoethanol,
(11) S-3-(1-hydroxy-2-N-methylamino-ethyl)-pyridine,
(12) S-3-(1-hydroxy-2-N-methylamino-ethyl)-thiophene,
(13) R-2-(1-hydroxy-2-N-methylamino-ethyl)-pyrazine,
(14) R-2-(1-hydroxy-2-N-methylamino-ethyl)-thiazole,
(15) R-2-(1-hydroxy-2-—-methylamino-ethyl)-thiophene,
(16) (R)-1-(1-benzofuran-2-yl)-2-(methylamino)ethanol, or
(17) (S)-1-(1-benzofuran-2-yl)-2-(methylamino)ethanol.

54. An intermediate useful for the preparation of a compound of formula I which is:
1. N-(4-chlorobenzyl)-8-fluoro-1-{2-[(2-hydroxyethyl)amino]-2-oxoethyl}-6-(morpholin-4-ylmethyl)-4-oxo-1,4-dihydroquinoline-3-carboxamide
2. N-(4-chlorobenzyl)-1-(2-hydroxyethyl)-9-(morpholin-4-ylmethyl)-2,7-dioxo-2,3-dihydro-1H,7H-pyrido[1,2,3-de]quinoxaline-6-carboxamide
3. N-(4-chlorobenzyl)-9-(chloromethyl)-1-(2-hydroxyethyl)-2,7-dioxo-2,3-dihydro-1H,7H-pyrido [1,2,3-de]quinoxaline-6-carboxamide
4. N-(4-fluorobenzyl)-1-methyl-9-(morpholin-4-ylmethyl)-2,7-dioxo-2,3-dihydro-1H,7H-pyrido[1,2,3-de]quinoxaline-6-carboxamide
5. N-(4-fluorobenzyl)-1-methyl-9-(chloromethyl)-2,7-dioxo-2,3-dihydro-1H,7H-pyrido[1,2,3-de]quinoxaline-6-carboxamide

55. The formula I of claim 1 which is formula IB.

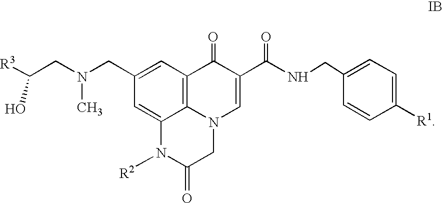

56. An intermediate useful for the preparation of a compound of formula I which is R-2-(1-hydroxy-2-N-methylamino-ethyl)pyridine.

57. An intermediate useful for the preparation of a compound of formula I which is N-methyl R-1-(2-furyl)-2-aminoethanol.

58. An intermediate useful for the preparation of a compound of formula I which is (R)-1-(1-benzofuran-2-yl)-2-(methylamino)ethanol.
